SEQUENCE LISTING (108 sequences)

SEQ ID NO: 1 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - protein, 385 aa, Cannabis sativa; identical to SEQ ID NO: 1

SEQ ID NO: 3 - protein, 249 aa, Pseudomonas putida
MILELDCGNSFIKWRVIHVADAVIEGGGIVDSDQALVAEVAALASVRLTGCRIVSVRSEEETDALCALIAQAFAVQAKVAHPVREMAGVRNGYDDYQRLGMDRWLAALGAFHLAKGACLVIDLGTAAKADFVSADGEHLGGYICPGMPLMRSQLRTHTRRIRYDDASAERALSSLSPGRSTVEAVERGCVLMLQGFAYTQLEQARVLWGEEFTVFLTGGDAPLVRAALPQARVVPDLVFVGLAMACPLD

SEQ ID NO: 4 - protein, 248 aa, Pseudomonas sp.
MILELDCGNSLIKWRVIEGAARSVAGGLAESDDALVEQLTSQQALPVRACRLVSVRSEQETSQLVARLEQLFPVSALVASSGKQLAGVRNGYLDYQRLGLDRWLALVAAHHLAKKACLVIDLGTAVTSDLVAADGVHLGGYICPGMTLMRSQLRTHTRRIRYDDAEARRALASLQPGQATAEAVERGCLLMLRGFVREQYAMACELLGPDCEIFLTGGDAELVRDELAGARIMPDLVFVGLALACPIE

SEQ ID NO: 5 - protein, 247 aa, Pseudomonas fragi
MILELDCGNSFIKWRVLDGGRVAGGGVVDSDIGLIASVRAVPALSVVQCRLVSVRTDEETASLVAGIASAFGVGVACAMPARSVSGVSNGYEDFSRLGLDRWLALVGGYELAKGACLVLDFGTAVTADFVAPDGSHLGGFICPGMPLMRDQLRTHTRRIRYDDFSAEQALLLRAPGRTTAEAVERGCLLMMRGFVLTQLELARDYWGQEFEVFLTGGDASLVSDMVPGARLVPDLVFVGLAVACPFS

SEQ ID NO: 6 - protein, 251 aa, Pseudomonas putida
MILELDCGNSFIKWRVIHAADATINVGGIVDSDQALVAAVAALSSLRLIGCRMVSVRSEEETSALCALILASFGVEVSVAQPVKEMAGVRNGYEDYQRLGMDRWLGALGAYHLAGRACLVIDFGTAAKADYVAADGEHLGGYICPGMPLMRSQLRTHTRRIRYDDASAERALGSLAPGRSTVEAVERGCVLMLQGFARTQIEQARALWGEDFTVFLTGGDAPLVQDAAPQARIVPDLVFVGLAMACPIESR

SEQ ID NO: 7 - protein, 246 aa, Pseudomonas stutzeri
MILELDCGNSFVKWRVISTGDGLVASEGIATQAIDLIQDLLDRNIGKITRCRLVSVRSDTETASIVGVLSESLKVSVATARPVERFAGVVNGYRNQQRLGLDRWLAILAAYDLRKAACLIIDLGTAITVDLVADDGVHLGGYITPGIALLRGQLVDHTRRIRYDEAEAALALSDVAPGRSTAEAVERGCLIMVRSYLSSQISSARRQLGNDFAVYVTGGDAAIAADLPGVRYMPDLVFRGLAIACP

SEQ ID NO: 8 - protein, 250 aa, Pseudomonas oryzihabitans
MILELDCGNSLIKWRLLAKGGDVVATGIVDSDEALIAALSQPDTPALTACRLVSVRSDEETQRLKELLTATFGIVCHQALPVEIWNGVRNGYRDHQRLGMDRWLVILAAVQLAEGKACVVLDLGTAVTADLVDAQGEHQGGFICPGIPLMRRELQTHTRRIRYDPHWASGERYEAPGRTTIEAVERGCLYMLKGFVRTQYELAQDMFAEGCEVFLTGGDAELVKHVVPGARIVPDMVFAGLALACPLENE

SEQ ID NO: 9 - protein, 249 aa, Pseudomonas otitidis
MILELDCGNSFIKWRLLGTDSEGALISGVAADGDNVIQAIKASDYSGIQQCRLVSVRSEEETSALLQALEQAFHARPLLASSSKVLAGVVNGYEDFHKLGLDRWLAVVAAYHVSKSASLVLDLGTAVTSDFVTSSGVHLGGYICPGVPLMRSQLRTHTRRIRYDDEAAAQAVAHMKPGRNTAEAVERGCLLMLKGFVRMQLDLARQQLGEDFCIFLTGGDAALVKEEVPNARLAPDLVFSGLAIACPFD

SEQ ID NO: 10 - protein, 249 aa, Pseudomonas brassicacearum
  misc_feature (89)..(89): Xaa can be any naturally occurring amino acid
MILELDCGNSFIKWRVLRADGVTTVDGGVVSSDSDLLVSLSNAEKFQLKKCRLVSVRTPEETDSLVASLVDAYGVSVTRAMPAREMAGXKNGYQDYERLGLDRWLALLGGFHLASGACLVLDFGTAITADFVAADGEHLGGFICPGMPLMRNQLRTHTRRIRYDDMAAERAHESLAPGRTTVEAVERGCMLMLRGFVLTQLELAQQYWGKDFVVFLTGGDANLVSGVVPDARVVPDLVFVGLAMACPLS

SEQ ID NO: 11 - protein, 652 aa, E. coli
MSQIHKHTIPANIADRCLINPQQYEAMYQQSINAPDTFWGEQGKILDWITPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSSSRLVITSDEGVRAGRSIPLKKNVDDALKNPNVTSVEHVVVLKRTGGKIDWQEGRDLWWHDLVEQASAQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 12 - protein, 652 aa, Salmonella typhimurium
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDASQSKHISYRELHRDVCRFANTLLDLGIKKGDVVAIYMPMVPEAAVAMLACARIGAVHSVIFGGFSPEAIAGRIIDSSSRLVITADEGVRAGRSIPLKKNVDDALKNPNVTSVEHVIVLKRTGNDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 13 - protein, 572 aa, Bacillus subtilis
MNLKALPAIEGDHNLKNYEETYRHFDWAEAEKHFSWHETGKLNAAYEAIDRHAESFRKNKVALYYKDAKRDEKYTFKEMKEESNRAGNVLRRYGNVEKGDRVFIFMPRSPELYFIMLGAIKIGAIAGPLFEAFMEGAVKDRLENSEAKVVVTTPELLERIPVDKLPHLQHVFVVGGEAESGTNIINYDEAAKQESTRLDIEWMDKKDGFLLHYTSGSTGTPKGVLHVHEAMIQQYQTGKWVLDLKEEDIYWCTADPGWVTGTVYGIFAPWLNGATNVIVGGRFSPESWYGTIEQLGVNVWYSAPTAFRMLMGAGDEMAAKYDLTSLRHVLSVGEPLNPEVIRWGHKVFNKRIHDTWWMTETGSQLICNYPCMDIKPGSMGKPIPGVEAAIVDNQGNELPPYRMGNLAIKKGWPSMMHTIWNNPEKYESYFMPGGWYVSGDSAYMDEEGYFWFQGRVDDVIMTSGERVGPFEVESKLVEHPAIAEAGVIGKPDPVRGEIIKAFIALREGFEPSDKLKEEIRLFVKQGLAAHAAPREIEFKDKLPKTRSGKIMRRVLKAWELNLPAGDLSTMED

SEQ ID NO: 14 - protein, 634 aa, Methanobacterium thermoformicicum
MSKDTSVLLEEKRVFKPHYTVVEEAHIKNWEAELEKGKDHENYWAEKAERLEWFRKWDRVLDESNRPFYRWFVNGKINMTYNAVDRWLDTDKRNQVAILYVNERGDERKLTYYELYREVSRTANALKSLGIKKGDAVALYLPMCPELVVSMLACAKIGAVHSVIYSGLSVGALVERLNDARAKIIITADGTYRRGGVIKLKPIVDEAILQCPTIETTVVVKHTDIDIEMSDISGREMLFDKLIEGEGDRCDAEEMDAEDPLFILYTSGSTGKPKGVLHTTGGYMVGVASTLEMTFDIHNGDLWWCTADIGWITGHSYVVYGPLLLGTTTLLYEGAPDYPDPGVWWSIVEKYGVTKFYTAPTAIRHLMRFGDKHPKRYNLESLKILGTVGEPINPEAWMWYYRNIGREKCPIIDTWWQTETGMHLIAPLPVTPLKPGSVTKPLPGIEADVVDENGDPVPLGKGGFLVIRKPWPAMFRTLFNDEQRYIDVYWKQIPGGVYTAGDMARKDEDGYFWIQGRSDDVLNIAGHRIGTAEVESVFVAHPAVAEAAVIGKADPIKGEVIKAFLILKKGHKLNAALIEELKRHLRHELGPVAVVGEMVQVDSLPKTRSGKIMRRILRAREEGEDLGDTSTLEE

SEQ ID NO: 15 - protein, 743 aa, Arabidopsis thaliana
MKIGSPSSPILSVVSSSGSLDPKISGSLGSRILPATQRSSPSENLLLHRKMSSNSLRHVESMSQLPSGAGKISQLNAVVLGESLASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWITGHSYVTYGPMLNGATVVVFEGAPNYPDPGRCWDIVDKYKVSIFYTAPTLVRSLMRDDDKFVTRHSRKSLRVLGSVGEPINPSAWRWFFNVVGDSRCPISDTWWQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 16 - protein, 713 aa, Saccharomyces cerevisiae
MSPSAVQSSKLEEQSSEIDKLKAKMSQSAATAQQKKEHEYEHLTSVKIVPQRPISDRLQPAIATHYSPHLDGLQDYQRLHKESIEDPAKFFGSKATQFLNWSKPFDKVFIPDPKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSSWSTATDDELQDIKKHLVFTVRKDIGPFAAPKLIILVDDLPKTRSGKIMRRILRKILAGESDQLGDVSTLSNPGIVRHLIDSVKL

SEQ ID NO: 17 - protein, 701 aa, Homo sapiens [sequence omitted]

SEQ ID NO: 18 - protein, 675 aa, Bos taurus
MAARCLGRGVGRLLGGLRGVGVRALAAPRLWSSASASVAPPSSYQERIALAAREPAAFWGPLARDALVWDTPYHTVSDCDFRSGRISWFLGGQLNVSVNCLDQHVQKSPESIALIWERDEPGTEVKITYRELLETTCRLANTLKRYGVRRGDRVAIYMPVSPLAVAAMLACARIGAIHNVIFAGFSVGSLAGRINDAQCKVVITFNQGLRGGRVVQLKKIVDEAIKVCPSVQHVLVAHRTDNKVHMGHLDVSLEQEMAKEEPVCAPESMGSEDILFLLYTSGSTGKPKGLVHTQAGYLLYAALTHRLVFDYRPGDIFGCVADIGWITGHSYVVYGPLCNGATSVLFESTPVYPDAGRYWETVQRLKINQFYGAPTAYRLLLKFEDSWVKKYDRSSLRTLGSVGEPINHEAWEWLHRVVGDSRCTLVDTWWQTETGGICISPRPSEEGAEILPCMAMRPLFGIVPVLMDEKGNVLEGGDVSGALCLSQAWPGMARTIYGDHQRFLDAYFETYPGYYFTGDGAYRTEEGYYEITGRMDDVINISGHRLGTAEIEDAMADHPAVPETAVIGYPHDIKGEAAFAFVVLKDDVGDVDVVVKELRSVVADKIAKYAVPDQVLVVKRLPKTRSGKVMRRLLRKIVMGRAQDLGDTTTLEDPGVITEILSAYQEYKDKRGGAK

SEQ ID NO: 19 - protein, 682 aa, Mus musculus
MAARSLGSGVGRLLRGLQGRSGQSGWSLSVSRSTATRLPGCVPAAAQPGSYPALSAQAAQEPAAFWGPLARDTLVWDTPYHTVWDCDFRTGKIGWFLGGQLNVSVNCLDQHVQKSPETIALIWERDEPGTEVRITYRELLETTCRLANTLKRHGVHRGDRVAIYMPVSPLAVAAMLACARIGAIHTVVFAGFSAESLAGRINDAKCKAVITFNQGLRGGRVVELKKIVDEAVKSCPTVQHVLVAHRTDTKVPMGSLDIPLEQEMAKEAPVCTPESMSSEDMLFMLYTSGSTGTPKGLVHTQAGYLLYAAMTHKLVFDYQPGDVFGCVADIGWITGHSYVVYGPLCNGATTVLFESTPVYPDAGRYWETVQRLKINQFYGAPTAVRLLLKYGDAWVKKYDRSSLRTLGSVGEPINHEAWEWLHKVVGDGRCTLVDTWWQTETGGICIAPRPSEDGAEILPGMAMRPFFGIVPVLMDEKGNVLEGGDVSGALCISQAWPGMARTIYGDHQRFVDAYFRAYPGYYFTGDGAHRTEGGYYQITGRMDDVINISGHRLGTAEIEDAMADHPAVPETAVIGYPHDIKGEAAFAFIVLKDNISDENMVVNELKLSVATKIAKYAVPDQILVVKRLPKTRSGKVMRRLLRKIITSRGQDLGDTTTLEDPSVITEILSAFQKYEEQRAATN

SEQ ID NO: 20 - protein, 542 aa, Ruegeria pomeroyi
MTGSTIYDQHLDRTPANFTPLSPLSYIERTAAVYPDYPSVVYGDRRYTWAETYTRCRRLASALAGRGLGKGDTVSIIAANIPEMYEAHFGVPMAGAVLNAINTRLDAPIIAFILSHAEARVLIVDPEFSEVVRDALAQIDRPDLLVVDIEDPSFAGGAPVGTLSYDALLAEGDPDFDWSLPGDEWDAIALNYTSGTTGNPKGVVYHHRGAALNATSNILTWGMPQHAVYLWTLPMFHCNGWCFPWTMAANAGVSVCLRAVRDEPIYRAFREEKVTHFCGAPIVLNMLANAPDHMKDFDHQIKVMTAGAPPPAAVIEKMEAMGVDVTHVYGLTETYGPSVVCAWKEEWDGRPGAERAALKVRQGVRYVALSGLMVADPETLEPVPADGETMGEIFMQGNIVMKGYLKNPDATDRAFRGGWFASGDLGVMHPDGYIALKDRSKDIIISGGENISSVEVEDVLYKHPAVMEAAVVARPDEKWGETPCAFVELKPGQSVEAADLIAHCRANMAHFKAPKTVVFGELPKTSTGKIQKFLLRERARAL

SEQ ID NO: 21 - protein, 589 aa, Burkholderia sp.
MLLTGKLADIVSPRRAGRSKPPARRRAVCSRPYRTNTSGRRKTMGKPLLGQMMDMPLLVSSLIAHAARHAGDVEIVSRRVEGDIHRYTYRDCEARSKRLAQALTRLGVGVGDRIGTLAWNGYRHVEAYYGISGMGAVCHTINPRLFPEQIAYIVNHAEDRYVLFDLTFAPLVDQLAPQCPNVKGWIAMTDDAHLPRGATPYLCYETLVGAQDGDYAWPLLDERQASSLCYTSGTTGHPKGALYSHRSTVLHAYGAALPDAMGLSSRDAALPVVPMFHVNAWGLPYTAALTGTKLVLPGKDLDGKSLYELIESERVTFSAGVPTVWLGLLAYMREAGVRFSTLDRTVIGGSACPPSMLETFEDVYDVRVIHAWGMTELSPLGTLSKLNWAQSQRSVDEQRRLLEKQGRVIYGIDMRIVGDDGRELPWDGVAFGDLQVCGPWVIDRYFGIDASPLVDGWFPTGDVATIDADGFLQITDRSKDVIKSGGEWISSIDVENVAVAHPAVAEAACIACAHPKWTERPLLVVVKRAGMDVTRGELLAFYDGKVAKWWIPDDVVFVDALPHTATGKLQKLKLREQFRDHVLPTAVDA

SEQ ID NO: 22 - protein, 542 aa, Ruegeria marina
MTGINIYDQNLDRTPANFTPLSPLSMIERTAAIYPDYPSVVYGARRYTWAETYARCRRLAGALVARGIGKGDTVSIIAANIPEMYEAHFGVPMAGAVLNAINTRLDAPIIAFILTHAEAKVLIVDPEFSAVVRDALDQIDRPDLLVVDIEDASFEGGARIGALTYEELLAEGDPEFAWSLPDDEWDAIALNYTSGTTGNPKGVVYHHRGAALNANTNILDWSMPKHSVYLWTLPMFHCNGWCFPWTMAANAGVSVCLRAVRDEPIYRAFREEKVTHFCGAPIVLNMLANAPAHLKDFDHQIKVMTAGAPPPAAVIEKMEAMGVEVTHVYGLTETYGPSVVCAWKEVWNARPAGERAALKVRQGVKNAALAGLMVADPDTLEPVPADGETMGEIFMQGNVVMKGYLKNPDATDKAFRGGWFASGDLGVMHPDGYVALKDRSKDIIISGGENISSVEVEGVLYKHPAVLEAAVVAKPDEKWGETPCAFVELKPGMAAEAAEIIAFCRDNMAHFKAPRTVVFGELPKTSTGKVQKFVLRDRARAL

SEQ ID NO: 23 - protein, 542 aa, Rhodobacteraceae bacterium KLH11
MTTATMYQQHLDKTPANFTPLSPLSYIERTAAIYPDYPSVVYGERRYTWADTYARCRRLASALVRRGVGAGDTVSIIAANIPALYEAHFGVPMAGAVLNAINTRLDAPIITFILNHAEAKVLLVDPEFSGVVKQALAQVDHDVLVIDIEDPDFDGGERLADLTYEALLAEGDPEFNWSLPGDEWDAITLNYTSGTTGNPKGVVYHHRGAALNATSNILTWGMPQHSIYLWTLPMFHCNGWCFPWTMAANAGTSVCLRAVRDVPIYRAFRDEGVTHFCGAPIVLNMLANAPDHLRDFSQQIKVMTAGAPPPAKVIEAMEGMGIEVTHVYGLTETYGPSVVCAWKDEWNEQDPEDRAALKIRQGVKYVALSGLMVADPKTLQPVPADGETMGEIFMQGNIVMKGYLKNPDATDKAFNGGWFASGDLGVMHPDGYIALKDRSKDIIISGGENISSVEVEDILYKHPAVMEAAVVARPDEKWGETPCAFVELKPQAEASADEIIAFCRDNMAHFKAPKTVVFGALPKTSTGKIQKFKLRDQARDLT

SEQ ID NO: 24 - protein, 542 aa, Ruegeria atlantica
MTQASIYERHLDKCRANYTPLSPLSFIERTAHIYPDYPSVVYGARRYTWAETYARCRRLAGALSARGISTGDTVSVIAANIPEMYEAHFGVPMAGAVLNAINTRLDAPIIAFILNHAESKVLLVDPEFSGVVKEALKQVDHDVLIVDIEDPSFGGGEKLGGLTYDALLAEGDPEFNWSLPKEEWDAITLNYTSGTTGNPKGVVYHHRGAALNAQSDLLDWNMPKHSVYLWTLPMFHCNGWCFPWAMAANAGVSVCLRAVRDEPIYAAFRDEKVTHFCGAPIVLNMLANAPDHLKDFDHEIKVMTAGAPPPAAVIESMENMGVEVTHVYGLTETYGPSVVCAWKDEWNDKGAEDRAALKIRQGVRYTALSGLMVADPDTLEPVPSDGETMGEIFMQGNVVMKGYLKNAEATDKAFRGGWFASGDLGVMHPDGYIALKDRSKDIIISGGENISSVEVEDILYKHPAVMEAAVVARPDDKWGETPCAFVELKPNAQASDADIIGFCRENMAHFKAPKTVVFGPLPKTSTGKIQKFKLRDQAKALT

SEQ ID NO: 25 - protein, 547 aa, Thioclava arenosa
MQTKHSIYESGLEVTAANYAALTPLSFAERTAAIYPELTAVVYGDLRRTWGETYERMCRIASALAGRGLGKDDTVSVIAANIPEMFEAHFAVPMIGAVLNTVNTRLDAEAIAFILSHAEAKAVLVDPEFSDVVERAIKISGRRDMLVIDIEDASYDGGHRIGSVTYDELLAEGDPEFRYVPPESEWDAISLNYTSGTTGNPKGVVYHHRGAALNAMSNIMTWGMAHHSRYLWTLPMFHCNGWCFPWTIAANAGVCVCLRAVRAEPIYEAIRNEKVTHFCGAPIVLNMLANAPDELKDFSHQVKVMTAGASPPAAVIEKMEAMGVEVTHVYGLTETYGPSVVCAWKGAWNARTAEERARLKARQGVRNLALAGLMVADPETLMPVPSDGETLGEIFMRGNNVMKGYLKNPTATDESFNGGWFASGDLGVMHPDGYVELKDRSKDIIISGGENISSVEVEDVLYKHPAVMEAAVVARPDPKWGETPCAFVELKPGQELEEAELIAFCRQNMAHFKTPKTVVYGPLPKTSTGKIQKFLLRDQARALNETK

SEQ ID NO: 26 - protein, 548 aa, Rhizobium selenitireducens
MQGPVTTSGDGIYDRELDKNAANFSQMTPLSFIERAAAVYPGRPAVVYGEVRRNWAQTYDRVRRLASALSKRGIGKGDTVAVIAANIPEMFECHFAVPMVGAVLNAINTRLDAEAIAFILNHGEALLLIVDPEFSEVVERAVHIAGRPIEVIDIIDPSFEGGDRLGAATYDELLADGNADFKWERPADEWDAISLNYTSGTTGDPKGVVYHHRGAYLNAIGNILTWNMANHPVYLWTLPMFHCNGWCFPWTIAAAAGVSVCLRAVRVEPIYELIKSEKVTHFCGAPIVLNLIAHAPDELKTGIDHKVSVMTAGAAPPASVIQAMEELGFDVTHTYGLTETYGPAVVCAWHDEWNGLEISEKARLKARQGVRYSVLDGLMVADPETLEPVPSDGETMGEIFFRGNNVMRGYLKNPSSSAKAFSGGWFHSGDLAVCHPDGYIEIKDRSKDIIISGGENISSIEVEGVLYRHPAVMEAAIVARPDEKWGETPCAFVGLKPGATVTEQELIAFCRANMAHFKAPKTVVFGPLPKTSTGKIQKYVLREKAKAL

SEQ ID NO: 27 - protein, 567 aa, Ralstonia solanacearum
MNDVAPAARDFLAARDLLLRYRTDYDRAYREFRWPELDTFNWALDYFDVIARGNDQPALWIVDDPRGEGLRLSYAQMSERSSRVANFLRGLGIVRGDRLLLMLPNRVELWDVMLAAMKLGAVVLPATTQLSADDLRDRVQLGGATCVVADAAELAKFEAVDAGVRRIAVGARRDGWIDLAEAYRAPAAFVPDGPTRATDPLLLYFTSGTTSKPKLVEHTHQSYPVGHLSTLYWIGLQPGDVHWNISSPGWAKHAWSCVFAPWNAQACVFVYNYARFVPRDTLEVLVRCNVTTLCAPPTVWRMLVQEPLASYPVKLREIVGAGEPLNPEIIERVRQAWGVTIRDGFGQTETTCQIGNTPGQPVVPGSVGRPLPGYRVELLDADDRPAAEGEIALPLSHRPLGLMQGYANHAKATAEAMRNGYYHTSDVAMRRDDGYFVYVGRTDDVFKSSDYRLSPFELESVLIEHEAIAEAAVVPSADPLRLSVPKAFVTLRQGYEAGPELARAVFRFSREKLAPYKRIRRLQFSELPKTISGKIRRVELRRREQERLPEPARLPGEYWEDDFADPR

SEQ ID NO: 28 - protein, 652 aa, Salmonella enterica
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGVVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 29 - protein, 582 aa, Candidatus Frankia californiensis
MTSQVLGTDGLAEFRAVRDFLLTHREDYDGAYAGFRWPQLAEFNWALEWFDHLGADPDSADRVALWIVEQDGSQRRQTFAQLSARSNQVANWLRERGVARGDRIVLMLGNQIELWETMLAAMKLGAVVIPATTLLAPADLRDRITRGQARYVVVGATDTDKFADVPGAYDRIAVGAPVEGWAHYADSAQASTVFVPDGVTHASDPLLLYFTSGTTAAPKLVEHTHVSYPVGHLSTMYWIGLTPGDVHLNISSPGWAKHAWSNVFAPWNAGATVLVYNYSRFDAAAMLAQIDRCGVDSFCAPPTVWRMLIQADLAVVRNPPRKVVGAGEPLNPEVIEQVEKAWGVRIRDGFGQTETTVQIGNSPGQLVKPGSMGRPVPGYRVTLVDVATGQPADEGEICLDLARRPLGLMVGYRDDAVRNAEAMAGGYYHTGDVASRDADGYITYIGRADDVFKASDYRISPFELESVLIEHPAVAEAAVVPSPDAIRLAVPKAYVVLADGYEPDRATALSILRHARQQLAPYKRIRRLEFAELPKTISGKIRRVELRRREDTLHPLSATAVPAGTSAPNREYRDEDFLELRE

SEQ ID NO: 30 - protein, 672 aa, Rattus norvegicus
MSKLARLEREEIMECQVMWEPDSKKDTQMDRFRAAVGTACGLALGNYDDLYHWSVRSYSDFWAEFWKFSGIVCSRMYDEVVDTSKGIADVPEWFRGSRLNYAENLLRHKENDRVALYVAREGREEIAKVTFEELRQQVALFAAAMRKMGVKKGDRVVGYLPNSAHAVEAMLAAASIGAIWSSTSPDFGVNGVLDRFSQIQPKLIFSVEAVVYNGKEHGHLEKLQRVVKGLPDLQRVVLIPYVLPREKIDISKIPNSMFLDDFLASGTGAQAPQLEFEQLPFSHPLFIMFSSGTTGAPKCMVHSAGGTLIQHLKEHVLHGNMTSSDILLYYTTVGWMMWNWMVSALATGASLVLYDGSPLVPTPNVLWDLVDRIGITILGTGAKWLSVLEEKDMKPMETHNLHTLHTILSTGSPLKAQSYEYVYRCIKSTVLLGSISGGTDIISCFMGQNSSIPVYKGEIQARNLGMAVEAWDEEGKTVWGASGELVCTKPIPCQPTHFWNDENGSKYRKAYFSKYPGVWAHGDYCRINPKTGGIVMLGRSDGTLNPNGVRFGSSEIYNIVEAFDEVEDSLCVPQYNRDGEERVVLFLKMASGHTFQPDLVKHIRDAIRLGLSARHVPSLILETQGIPYTINGKKVEVAVKQVIAGKTVEHRGAFSNPESLDLYRDIPELQDF

SEQ ID NO: 31 - protein, 632 aa, Ralstonia solanacearum
MPMSDAYRALYQRSIDDPAAFWGEQAQRIDWQTPYAAVLDDARLPFARWFVGGRTNLCHNAVDRHLATRGEQAALVYVSTETGIETTYTYRALHREVNRMAACLQALGVRRGDRVLIYLPMIPEAAFAMLACARIGAIHSVVFGGFASNSLATRIDDATPRVIVSADAGSRGGKVVEYKPLLDAAIDLAVHKPAHVLLVDRKLAPMQHRPHDIDYAALARQHTHADVPCEWMESNEPSYILYTSGTTGKPKGVQRDTGGYAVALAASMPLIFGAQAGDTMFTASDVGWVVGHSYIVYAPLLAGLATVMYEGTPVRPDGAIWWRIVEQYRVNVMFTAPTAIRVLKKQDPALLRRHDLSSLRRLFLAGEPLDEPTARWIGDALGKPIIDNYWQTETGWPMLAIPQGVAPSTPKLGSPGFPVYGYRLDILDEATGQPCAPGEKGLLAVAAPLPPGCMSTVWGDDARFLKTYWSAFPGRPLYSSFDWGVRDEAGYITILGRTDDVINVAGHRLGTREIEESLSSHPAIAEVAVVGVADPLKGQVAMGFAIVRDAARVAEPAGRMALEGELMRTVEGQLGAVARPSRVFFVNALPKTRSGKLLRRAMQAVAEGRDPGDLTTIEDPTALAQVREAMQA

SEQ ID NO: 32 - protein, 628 aa, Salmonella enterica
MSFSEFYQRSINEPEAFWAEQARRIDWRQPFTQTLDHSRPPFARWFCGGTTNLCHNAVDRWRDKQPEALALIAVSSETDEERTFTFSQLHDEVNAVAAMLLSLGVQRGDRVLVYMPMIAEAQITLLACARIGAIHSVVFGGFASHSVAARIDDARPALIVSADAGARGGKILPYKKLLDDAITQAQHQPKHVLLVDRGLAKMAWVDGRDLDFATLRQQHLGASVPVAWLESNETSCILYTSGTTGKPKGVQRDVGGYAVALTTSMDTIFGGKAGGVFFCASDIGWVVGHSYIVYAPLLAGMATIVYEGLPTYPDCGVWWKIVEKYQVNRMFSAPTAIRVLKKFPTAQIRNHDLSSLEVLYLAGEPLDEPTASWVTETLGVPVIDNYWQTESGWPIMALARALDDRPSRLGSPGVPMYGYNVQLLNEVTGDPCGINEKGMLVIEGPLPPGCIQTIWGDDARFVKTYWSLFNRQVYATFDWGIRDAEGYYFILGRTDDVINIAGHRLGTREIEESISGYPNVAEVAGGGVKDALKGQVAVAFVIPKQSDTLADREAARDEEKAIMALVDNQIGHFGRPAHVWFVSQLPKTRSGKMLRRTIQAICEGRDPGDLTTIDDPASLQQIRQAIEE

SEQ ID NO: 33 - protein, 504 aa, Rhizobium leguminosarum
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 34 - protein, 504 aa, Rhizobiales bacterium
MSNHLFDAIRAAAPGDAPFIRVDSKRTWTYNDALALSGRMASAMDTLGIRPGDRVAVQVEKSAEALILYLACLRSGAVYLPLNTAYTLAELDYFIGDAEPRLVVVAPSAKTGIEEMAKVHGVIVETLDADGSGSLLDLARDEPADFVDASRSAADLAAILYTSGTTGRSKGAMLTHGNLLSNALTLRDYWRVTAADRLIHALPIFHTHGLFVATNVTMLAGASMFLLSKFDADEVVSLMPQATLLMGVPTFYVRLLQSPRLNREAVANIRLFISGSAPLLAETHTEFQARTGHAILERYGMTETNMNTSNPFDGKRIPGTVGFPLPGVTVRVTDPATGVLLGPDETGMIEIKGPNVFKGYWRMPEKTAAEFTTDGFFISGDLGKIDRDGYVHIVGRGKDLVISGGYNIYPKEVEGEIDLIDGVIESAVIGVPHPDFGEGVTAIVVRKPGATVDEKAIVGALQDRLARYKQPKRIIFTDDLPRNTMGKVQKNVLRQQYADLYART

SEQ ID NO: 35 - protein, 504 aa, Rhizobium phaseoli
MSNHLFDAMRAAAPGDAPFMRTGSGQTWTYDDAFALSGRIAGAMDTLGIRPGDRVAVQVEKSAAALILYLACLRSGAVYLPLNTAYTLAELDYFIGDAEPRLVVVASAAREGVDTVAKRHGAIVETLDADGSGSLLDLARDEPADFVDASRSADDLAAILYTSGTTGRSKGAMLTHGNLLSNALSLRDYWRVTADDRLIHALPIFHTHGLFVATNVTLLAGASMFLLAKFDADEVVSLMPEATMLMGVPTFYVRLLQSPRFGKEAAANIRLFISGSAPLLADTHTEFEARTGHAILERYGMTETNMNTSNPYDGRRIAGTVGLPLPGVTVRVTDPATGQVLPPEQTGMIEIKGPNVFKGYWRMPEKTAAEFTGDGFFISGDLGKIDSDGYVHIVGRGKDLVISGGYNIYPKEVESEIDRIEGVVESAVIGVPHPDFGEGVTAVVVRKPGAVLDEKTIVSALQDRLARYKQPKRIIFAEDLPRNTMGKVQKNILRQQYAGLYTGT

SEQ ID NO: 36 - protein, 505 aa, Streptomyces coelicolor
MGSSHHHHHHSSGLVPRGSHMSSLFPALSPAPTGAPADRPALRFGERSLTYAELAAAAGATAGRIGGAGRVAVWATPAMETGVAVVAALLAGVAAVPLNPKSGDKELAHILSDSAPSLVLAPPDAELPPALGALERVDVDVRARGAVPEDGADDGDPALVVYTSGTTGPPKGAVIPRRALATTLDALADAWQWTGEDVLVQGLPLFHVHGLVLGILGPLRRGGSVRHLGRFSTEGAARELNDGATMLFGVPTMYHRIAETLPADPELAKALAGARLLVSGSAALPVHDHERIAAATGRRVIERYGMTETLMNTSVRADGEPRAGTVGVPLPGVELRLVEEDGTPIAALDGESVGEIQVRGPNLFTEYLNRPDATAAAFTEDGFFRTGDMAVRDPDGYVRIVGRKATDLIKSGGYKIGAGEIENALLEHPEVREAAVTGEPDPDLGERIVAWIVPADPAAPPALGTLADHVAARLAPHKRPRVVRYLDAVPRNDMGKIMKRALNRD

SEQ ID NO: 37 - protein, 485 aa, Streptomyces canus
MSSLFPALSPAPTGAPGDRPALRFGGRSLTYAELAAAAGATAGRIGGAGRVAIWATPAMETAVAVVAALLAGVPAVPLNPKSGDKELAHILSDSAPSLVLAPPDGELPPALVALERVDVDVRARGTVPEDRADDGDPALVVYTSGTTGPPKGAVIPRRALATTLDALADAWRWTGDDVLVQGLPLFHVHGLVLGILGPLRRGGSVRHLGRFSTEGAARELNDGATMLFGVPTMYHRIAETLPGDPELAKALSGARLLVSGSAALPVHDHERIAAATGRRVIERYGMTETLMNTSVRADGEPRAGTVGVPLPGVELRLVEEDGTPIAALDGESVGEIQVRGPNLFTEYLNRPDATAAAFTGDGFFRTGDMAVRDPDGYVRIVGRKATDLIKSGGYKIGAGEIENALLEHPGVREAAVTGEPDPDLGERIVAWIVPADPAAPPALDTLADHVAGRLAPHKRPRVVRYLDALPRNDMGKIMKRALDRD

SEQ ID NO: 38 - protein, 485 aa, Streptomyces luteus
MSPLFPALSPAATGAGADRPALRFGERSLTYGQLAAAAGGTADHIRGAGRVAVWATPAMETAVAVVAALLAGVPAVPLNPKSGDKELGHILSDSEPSLVLAPPGAELPPALGALERIDVDVHATGPVPEDRADDGAPALVVYTSGTTGPPKGAVIPRRALAATLDALADAWRWTDDDVLVQGLPLFHVHGLVLGILGPLRRGGSVRHLGRFTTEGAARELNDGATMLFGVPTMYHRIAEALPAEPELAKALAGARLLVSGSAALPVHDHERIAAATGRRVIERYGMTETLMNTSVRADGQPRAGTVGVPLPGVQLRLVEEDGTPIAALDGESVGEIQVRGPNLFTEYLNRPDATAAAFTEDGFFRTGDMAVRDPDGYVRIVGRKATDLIKSGGYKIGAGEIENALLEHPGVREAAVTGEPDPDLGERIVAWIVPADPASPPALETLADHVAGRLAPHKRPRVVRYVDALPRNDMGKIMKRALTDG

SEQ ID NO: 39 - protein, 608 aa, Arabidopsis thaliana
MTATTTLKSFNYLSLINHRLNHNSYAILSSPLLPRSHPASTSFSNSGFRFFQSNHLFSSQSGSLMEVFKAAFSEASNSCDRIAIKADGKSYSYGQLTSSALRISKLFLKDDTTNGGQETKKYEGFGSLKGARIGIVAKPSAEFVAGVLGTWFSGGVAVPLALSYPEAELLHVMNDSDISLLLSTEDHSETMKTIAAKSGARFHLIPPVVNSTSETVACNQFQDDSFEAEGKFLDDPALIVYTSGTTGKPKGVVHTHNSINSQVRMLTEAWEYTSADHFLHCLPLHHVHGLFNALFAPLYARSLVEFLPKFSVSGIWRRWRESYPVNDEKTNDSITVFTGVPTMYTRLIQGYEAMDKEMQDSSAFAARKLRLMMSGSSALPRPVMHQWESITGHRLLERYGMTEFVMAMSNPLRGARNAGTVGKPLPGVEAKIKEDENDANGVGEICVKSPSLFKEYWNLPEVTKESFTEDGYFKTGDAGRVDEDGYYVILGRNSADIMKVGGYKLSALEIESTLLEHPTVAECCVLGLTDNDYGEAVTAIIIAESAAKKRREDESKPVITLEELCGWAKDKLAPYKLPTRLLIWESLPRNAMGKVNKKELKKSLENQE

SEQ ID NO: 40 - protein, 614 aa, Camelina sativa
MTATALKSFNYSLSLITTHHRLNHNYYSYAILSCRRLPRFQPASTSSFLNSGFRFFHSNRHLFSSQSGSLMEVFKAACSEGSSSSCDRIAIKADGKSYSYGQLISSALSISKLFSKDNSLNGGETKKYEGFGSLKGARIGIVAKPSAEFVAGVLGTWFSGGVAVPLALSYPEAELLHVMNDSDISLLLSTEDHSETMKTIAAKSGAQYHLIPLVVDSSSETVASNQLQDNSFETEGKFLDEPALIVYTSGTTGKPKGVVHTHNSINSQVKMLTEAWEYTSADHILHCLPLHHVHGLFNALFAPLYARSSVEFLPKFSVSGIWRRWRESYPVNDEKTDDAITVFTGVPTMYTRLIQGYEAMDKEMQDSSASAARKLRLMMSGSSALPRPVMHQWESITGHRLLERYGMTEFVMAISNPLRGERKAGTVGKPLPGVEAKIKQDENDLNGVGEICVKSPSLFKEYWNLPEVTKESFTEDGYFKTGDAGRVDEDGYYVILGRNSADIMKVGGYKLSALEIESTLLEHPTVAECCVLGLTDKDYGEAVTAIIVAETEAKKRREDESKPVMTLEELSGWAKDKLAPYKLPTRLLIWESLPRNAMGKVNKKELKKSLDNQE

SEQ ID NO: 41 - protein, 605 aa, Brassica rapa
MTATLKSFNYFSYIINSRLHNSPILSYRRRRLPRSQPPTSCLNPGFRFFRPNHLFCTQSGSLMEVFKAVLSEGSNACDRIAIKADEKSYSYGQLTSSALTISKLFCREDTTNGGESKKYEGFGSLKGARVGIVAKPSAEFVAGVLGTWFSGGVAVPLALSYPEAELLYVMNNSDISVLLSTEDHSETMKTIAAKSDARFHLIPSVLNSTSETVTRNQFQDDSFEEDGKLLDDPALIVYTSGTTGKPKGVVHTHKSINSQVRMLTEAWEYTSADHFLHCLPLHHVHGLFNALFAPLYARSSVEFLPKFSVSGIWRRWRESYPVNDDKTDNPITVFTGVPTMYTRLIQGYEAMDQETKESSAFAAQKLRLMMSGSSALPRPVMHQWESITGHRLLERYGMTEFVMAISNPLRGERKAGTVGKPLPGVEAKIVQDKNDTDGVGEICVKSPSLFKEYWNLPEVTKESFTEDGYFKTGDAGRVDEDGYFVILGRTSADIMKVGGYKLSALEIESTLLEHPTVAECCVLGLPDKNYGEAVTAIIVAEAGAKRKRDDESKPVMTLEELCGWAKDKLAPYKLPTRLIIWESLPRNAMGKVNKKELKKSLDHQE

SEQ ID NO: 42 - protein, 448 aa, Rhizobium leguminosarum
MGIELLSIGLLIAMFIIATIQPINMGALAFAGAFVLGSMIIGMKTNEIFAGFPSDLFLTLVAVTYLFAIAQINGTIDWLVECAVRLVRGRIGLIPWVMFLVAAIITGFGALGPAAVAILAPVALSFAVQYRIHPVMMGLMVIHGAQAGGFSPISIYGGITNQIVAKAGLPFAPTSLFLSSFFFNLAIAVLVFFVFGGARVMKHDPASLGPLPELHPEGVSASIRGHGGTPAKPIREHAYGTAADTATTLRLNNERITTLIGLTALGIGALVFKFNVGLVAMTVAVVLALLSPKTQKAAIDKVSWSTVLLIAGIITYVGVMEKAGTVDYVANGISSLGMPLLVALLLCFTGAIVSAFASSTALLGAIIPLAVPFLLQGHISAIGVVAAIAISTTIVDTSPFSTNGALVVANAPDDSREQVLRQLLIYSALIAIIGPIVAWLVFVVPGLV

SEQ ID NO: 43 - protein, 449 aa, Rhizobium phaseoli
MGIEILAIGLLIAMFIVATIQPINMGALAFAGAFVLGSMTIGMKTTDIFAGFPSDLFLTLVAVTYLFAIAQINGTIDWLVECAVRLVRGRIGLIPWVMFLVAAVITGFGALGPAAVAILAPVALSFAIQYRIHPVMMGLMVIHGAQAGGFSPISIYGGITNQIVAKAGLPFAPTSLFLSSFFFNLAIAVLVFFAFGGAKVMKHVPAASFGPLPELHPEGVSASIRGHGGTPAKPIREHAYGTAADTAATLRLNNERITTLIGLTALGIGALVFKFNVGLVAMTVAVALALLSPKTQKAAIDKVSWSTVLLIAGIITYVGVMEKAGTVDYVANGISSLGMPLLVALLLCFTGAIVSAFASSTALLGAIIPLAVPFLMQGHVSAIGVVAAIAISTTIVDTSPFSTNGALVVANAPDDRRDQVLKQLLVYSALIAIIGPIVAWLVFVVPGLV

SEQ ID NO: 44 - protein, 465 aa, Streptomyces coelicolor
MSPELISILVLVVVFVIATTRSVNMGALAFAAAFGVGTLVADLDADGIFAGFPGDLFVVLVGVTYLFAIARANGTTDWLVHAAVRLVRGRVALIPWVMFALTGALTAIGAVSPAAVAIVAPVALSFATRYSISPLLMGTMVVHGAQAGGFSPISIYGSIVNGIVEREKLPGSEIGLFLASLVANLLIAAVLFAVLGGRKLWARGAVTPEGDGAPGKAGTGTTGSGSDTGTGTGTGTGTSAGTGGTAPTAVAVRSDRETGGAEGTGVRLTPARVATLVALVALVVAVLGFDLDAGLTAVTLAVVLSTAWPDDSRRAVGEIAWSTVLLICGVLTYVGVLEEMGTITWAGEGVGGIGVPLLAAVLLCYIGAIVSAFASSVGIMGALIPLAVPFLAQGEIGAVGMVAALAVSATVVDVSPFSTNGALVLAAAPDVDRDRFFRQLMVYGGIVVAAVPALAWLVLVVPGFG

SEQ ID NO: 45 - protein, 460 aa, Streptomyces pactum
MSPELISILVLVVVFVIATTRSVNMGALAFAAAFGVGTVVADLDADGIFAGFPGDLFVVLVGVTYLFAIARANGTTDWLVHAAVRLVRGRVALIPWVMFALTGALTAIGAVSPAAVAIVAPIALSFATRYAISPLLMGTMVVHGAQAGGFSPISIYGSIVNGIVEREGLPGSEITLFLASLIANLVIASVLFVLFGGRKLWARGSVATEDAGAAGKGTQPTGTGPDTGTAGPGAGSGSGGAAPTAVAVRPDQATDGTGGGLRLTPARIATLTALVALVVAVLGFDLDAGLTAVTLAVVLSTAWPNDSRRAVGEIAWSTVLLICGVLTYVGVLEEMGTITWAGEGVGGIGVPLLAAVLLCYIGAIVSAFASSVGIMGALIPLAVPFLAQGEIGAVGMVAALAVSATVVDVSPFSTNGALVLAAAPDVDRERFFRQLMIYGGIVVAVVPALAWLVLVVPGFG

SEQ ID NO: 46 - protein, 123 aa, Propionibacterium freudenreichii
MKLKVTVNGTAYDVDVDVDKSHENPMGTILFGGGTGGAPAPRAAGGAGAGKAGEGEIPAPLAGTVSKILVKEGDTVKAGQTVLVLEAMKMETEINAPTDGKVEKVLVKERDAVQGGQGLIKIG

SEQ ID NO: 47 - protein, 505 aa, Propionibacterium freudenreichii
MSPREIEVSEPREVGITELVLRDAHQSLMATRMAMEDMVGACADIDAAGYWSVECWGGATYDSCIRFLNEDPWERLRTFRKLMPNSRLQMLLRGQNLLGYRHYNDEVVDRFVDKSAENGMDVFRVFDAMNDPRNMAHAMAAVKKAGKHAQGTICYTISPVHTVEGYVKLAGQLLDMGADSIALKDMAALLKPQPAYDIIKAIKDTYGQKTQINLHCHSTTGVTEVSLMKAIEAGVDVVDTAISSMSLGPGHNPTESVAEMLEGTGYTTNLDYDRLHKIRDHFKAIRPKYKKFESKTLVDTSIFKSQIPGGMLSNMESQLRAQGAEDKMDEVMAEVPRVRKAAGFPPLVTPSSQIVGTQAVFNVMMGEYKRMTGEFADIMLGYYGASPADRDPKVVKLAEEQSGKKPITQRPADLLPPEWEEQSKEAAALKGFNGTDEDVLTYALFPQVAPVFFEHRAEGPHSVALTDAQLKAEAEGDEKSLAVAGPVTYNVNVGGTVREVTVQQA

SEQ ID NO: 48 - protein, 524 aa, Propionibacterium freudenreichii
MAENNNLKLASTMEGRVEQLAEQRQVIEAGGGERRVEKQHSQGKQTARERLNNLLDPHSFDEVGAFRKHRTTLFGMDKAVVPADGVVTGRGTILGRPVHAASQDFTVMGGSAGETQSTKVVETMEQALLTGTPFLFFYDSGGARIQEGIDSLSGYGKMFFANVKLSGVVPQIAIIAGPCAGGASYSPALTDFIIMTKKAHMFITGPQVIKSVTGEDVTADELGGAEAHMAISGNIHFVAEDDDAAELIAKKLLSFLPQNNTEEASFVNPNNDVSPNTELRDIVPIDGKKGYDVRDVIAKIVDWGDYLEVKAGYATNLVTAFARVNGRSVGIVANQPSVMSGCLDINASDKAAEFVNFCDSFNIPLVQLVDVPGFLPGVQQEYGGIIRHGAKMLYAYSEATVPKITVVLRKAYGGSYLAMCNRDLGADAVYAWPSAEIAVMGAEGAANVIFRKEIKAADDPDAMRAEKIEEYQNAFNTPYVAAARGQVDDVIDPADTRRKIASALEMYATKRQTRPAKKHGNFPC

SEQ ID NO: 49 - protein, 132 aa, C thermocellum
MKKFLIKVNGNQYEVEVEEIRDGASAPQVTLSTPSAAPAPSPAPAQETKTAAPKKDSTVPAGATAIKAPMPGTILDIRVNQGDTVKKGQVLLILEAMKMENEIVAPNDGTVASINVSKGASVNVGEVLVSLK

SEQ ID NO: 50 - protein, 465 aa, C thermocellum
MAKVKITETALRDAHQSLIATRMRIEEMLPIIDKLDEIGYHSLEVWGGATFDACLRFLNEDPWERLRIIKSHCKKTPLQMLLRGQNLLGYKHYADDVVEYFVQKSVANGINIIRIFDALNDTRNIETAIKACKKEGGHAQGTVCYTISPVHNLELFVKDAKTLVEMGADSICVKDMAGLLLPYVAYDLIKALKENVKVPIQLHTHYTSGVASMTYLKAIEAGCDVVDCAISPMSMGTSQPPTEPLVATLKGTPYDTGLDLDKLSEIADYFRPLKEKYISEGLLDVKVMGVDVNTLKYQVPGGMLSNLVSQLKQSNAVDKFEEVLKEVPRVREDFGYPPLVTPTSQIVGTQAVLNVVTGERYKMVPKESKALIKGEYGRTPAPVNPEVQKKILKDEEPITVRPADLIEPELDKIRNEMKEYLEQDEDVLSYALFPQVAEKFFQYRKAQKYKIEPDMVDYENRVHPV

SEQ ID NO: 51 - protein, 516 aa, C thermocellum
MDKVDKIGLLREKLAQVEQGGGAEKIAKQHDAGKMTARERIQALFDENSFVEIDTFVETRSIDFDMQKKKVPGDGVVTGYGSIDGRLVFVAAQDFTVIGGSLGEMHAAKITKVMDMAMKMGAPFISINDSGGARIEEGIDALKGFGDIFYRNTLASGVIPQISVIMGPCAGGAVYSPAITDFIFMVDKTSQMFITGPQVIKSVTGEDVTFEKLGGAETHNSISGVAHFRSSSEKECIEQIKKLISYLPDNNLSDVPIVPTQDDINRITDNLVDIIPQDSNKPYDMMEIITSVVDNGDFFEIQKDFAKNIIIGFGRMNGGTVGIVANQPKVAAGVLDVNSSDKAARFVRFCDAFNIPIITFTDVPGYLPGVGQEHSGVIRHGAKLLYAFSEATVPKINVIVRKAYGGAYIAMNSKHLGADMVFAWPSAEIAVMGPEGAANIIFKKDIAAADDPMETRKRLIEEYREKFSNPYVAASRGYVDDVIDPATTRIRLISALEMLASKRENRPAKKHGNIPL

SEQ ID NO: 52 - protein, 127 aa, T saccharolyticum
MKKFIVTVNGKKYDVEVEEVKVDVASEKKAKEDTAAKNASDASVKSKQVEVKNEVKDGFSINAPMPGTILDVKISQGQTVRRGDVLLILEAMKMENEITSPYDGTIISINVSKGASVNTGDVLLYLK

SEQ ID NO: 53 - protein, 464 aa, T saccharolyticum
MSKIKITETVLRDAHQSLLATRMTTDEMLPIAEKLDEVGFFSLEAWGGATFDACMRFLNEDPWERLRLLKKAIKKTPLQMLLRGQNLLGYKHYPDDVVNEFIIKSVENGIDIIRIFDALNDVRNLEVPIKSAKSAGAHVQAAIVYTVSPVHNTDHYLKVAKSLQDMGADSICIKDMSGILSPYVAYDLIKSLKRALYTPIQLHSHYTAGLASMTYLKAIEAGVDGVDTAISSLALGTSQPATESIVAALKDTEYDTGLDLKLLAEIAQHFNVVKQNHKNDSDMSLLMSVDVKALESQIPGGMLSNLVSQLKQQNALNKYQDVLKEVPRVREDLGYPPLVTPMSQMVGTQAVLNVITGERYKIVPKEIKDYVKGLYGMPPAPISDSIRKKIIGDEEVISKRPADLLSPQLDEFKNEIKEFIEQDEDVLSYALFPQVARRFFEYRQAKKYRIDSTLLNIEERVHPI

SEQ ID NO: 54 - protein, 516 aa, T saccharolyticum
MSIDDRIEDLLRRREMVLEGGGLDKVEKQHQKGKLTARERIYKLLDEDSFVEIDAYVEHRCIDFGMEKQRIPGEGVVTGYGTIDGRLVYVYAQDFTVLGGSLGEYHAKKITKIMDMALKMGAPLIGLNDSGGARIQEGVDALSGYGNIFFRNTLASGVIPQISVIMGPSAGGAVYSPALTDFIFMVDKTSQMFITGPQVIKAVTGEDVSAEELGGSITHSTKSGVAHFRAENDEECLKMVRKLLSYLPSNNLEDPPQLATDDDINRFSDRLIEIIPDSPNKPYDMKEVISEIVDEGVYFESQAMYAQNIITAFARLNGRTVGIIANQPKVLAGCLDINASDKASRFIRFCDAFNIPLLNIVDVPGFLPGTNQEYGGIIRHGAKMLYAYSEATVPKVTLIVRKAYGGAYLAMCSKDLGADFVLAWPTAEIAVMGPDGAANIVFKNEIKSSDDPVAARNEKINEYRENFANPYRAAARGYVDDVVLPQETRPRLISAFDMLMSKRESRPSKKHGNFPV

SEQ ID NO: 55 - protein, 127 aa, Caldicellulosiruptor beschii
MRKFKVKINSQEFVVEVEEIGVENATSVVPRPKIGHFEPKQEKHEDKTKQSPVLSSDKNSVVAQLPGTIVRLLKSEGDVVDANEPVLILEAMKMENEITAPVKGKIKRIHVKEGQKVAKGDLLFEIE

SEQ ID NO: 56 - protein, 463 aa, Caldicellulosiruptor beschii
MGVKITETILRDAHQSLIATRMTTEQMLEIAPVLDQVGYYSVECWGGATFDACLRFFNEDPWERLKRLRTAFKKTKLQMLLRGQNLVGYRHYSDDVVEEFVKKAIYYGIDIIRIFDALNDIRNIEMALKITKKEKGHAQVAISYTVSPYHTIENYVNLAKQIEELGADSICIKDMAGLLSPFDAYKLVKALKEQVKLPIHLHTHYTTGFGSMTYLKAVEAGVDGIDTALSPLALGTSQPPTETIVYALENTEYAPKLDLEKINEASEYFKVLREEYIRKGLLDPKVLSVDINALHYQIPGGMLSNLISQLKEQGQEDKLDEVLKEVPEVRKDFGYPPLVTPTSQIVGTQAVLNVIAGERYKLVTKETKAYFKGEYGKPPAPVNEEVKRKILKDEKEITCRPADLILPELENAKEKIKEYIENDTDVVTYCLFPQLAENFFKLRFAKKYKVDADLVQGNKVYPV

SEQ ID NO: 57 - protein, 514 aa, Caldicellulosiruptor beschii
MTNKLRELKQKRERILKLGGEDKIKKQHDSKKLTCRERIEYLLDPGSFNEIDMFVEHRCQEFDMKDTFVPCDGVVTGYGTINGRKVFVYAQDFTSIGGSLGEMHAKKICKVLDLALKYGCPVIGINDSGGARIQEGVDALAGYGEIFYRNTMASGVIPQIAAIMGPCAGGAVYSPAIMDFIFMVDKTSQMFVTGPQVIKAVTGEEISFEELGGAYTHSSKSGVAHFIAEDEYHLLDMIKYLLSFIPSNNMEDPPFIMSSDSEKRFVPELENIIPQEPNKAYDVKEIIYKVVDNQEFLEVQPYFAQNAVVGFGRIGGFSVGIVANQPKVNAGVLDYDSSDKIARFVRFCDAFNIPIITFTDVPGFLPGVNQEHNGIIRHGAKVLYAYSEATVPKINVILRKAYGGAYIAMSSKHIGADFVFAWPTAEIAVMGPDGAANIIFRKEIQSAQNPEEERKRRIEEYTQKFANPYIAAARGYVDDVIEPQLTRNKIIEALKISITKREQRPPKKHGNIPL

SEQ ID NO: 58 - protein, 123 aa, Corynebacterium kroppenstedtii
MKLTVTVNGVPYSVDVEVEHEERPTLGTIITGGNSNGPTPTAPTTSSVQGVSANSVTAPLAGSVSKVLVEEGQAITAGEVIVVLEAMKMETEITAPNDGTVTALHVQPGDAVQGGQSLLEIGD

SEQ ID NO: 59 - protein, 498 aa, Corynebacterium kroppenstedtii
MTTRKIGVTELALRDAHQSLMATRMALEDMVDACEDIDKAGYWSVECWGGATFDACIRFLNEDPWERLRTFRKLMPNSRLQMLLRGQNLLGYRHYEDGVVDKFVEKSAENGMDVFRVFDALNDPRNLEHAMQAVKKVGKHAQGTICYTVSPLHDVQGYIDLAGRLLDMGADSIALKDMAALLKPQPAYDIIRGIKDTYGEDTQINVHCHSTTGVTMVTLMKAIEAGADVVDTAISSMSLGPGHNPTESLVEMLEGTDYETGLDMDRLINIRDHFKTVRPKYAEFESKTLVNTNIFQSQIPGGMLSNMESQLKAQGAGDRIDEVMKEVPVVRKAAGYPPLVTPSSQIVGTQAVFNVLMGRYKVLTAEFADLLLGYYGEAPGERDKDLIEQAKKQTGKEPITERPADLLEPEWDNLVEEADELDGTDGSDEDVLTNALFPQVAPGFFKTRPDGPKNVGKTKEQLEREEAKASGDATAIREPIMYKVTTGGRSHTVSVEPA

SEQ ID NO: 60 - protein, 527 aa, Corynebacterium kroppenstedtii
MSEQPHDPSMPERLGQLEEERNRIRLGGGQARLDKQHDRGKMTARERITKLVDEDTFQETGMFAKHRTTHFGMDKADAPADGVVTGSGAVYGRPVHIASQDFSVMGGSAGEMQSNKVVAMMKASATTGTPFVFINDSGGARVQEGIDSLSGYGRVFYNNVLLSGLVPQVSIIAGPCAGGAAYSPALTDFIIQTRKANMFITGPKVIESVTGEKVTADELGGADAHMSTAGNIHFVADDDEQAILIAQKLLSFLPQNNTEEPPIVDPDEVVEPDDSLRDIVPVDGRKGYDVRDIIRKIVDYGDFLEVQAGYAQNLVVGFARVVGRTVGIVANQSQVMSGVLDINSSDKGASFVRFCDSFNIPLLTLVDVPGFMPGVAQEHGGIIRHGAKMLFAYSAATVPKLTVVLRKSYGGSYLAMCSKDLGADRVWAWPTAEIAVMGADGAVNVVFRKEIKKAQEEGGDEAAAAKKSELVQLYKDTFSTPYMAASRGLVDDIIDPADTRREIALALELLTNKRENRPSKKHGLAPN

SEQ ID NO: 61 - protein, 131 aa, Geobacter bemidjiensis
VQLTMTIDGKKYRVDVEVEEGEEVRTEGAFPPTATMQAYPVYSAHPTATPPLAAPTPASSSEKICRSPIAGVVFKIVAQVGQHLEMNDLLVVLEAMKMETNITAHMSGKVEKILVSVGEAVQPGQAIAEFA

SEQ ID NO: 62 - protein, 501 aa, Geobacter bemidjiensis
MDRIIDITELALRDAHQSLIATRLGIDDMVPVCEDLDQAGYWSIECWGGATYDACIRFLNEDPWVRLRTFKELMPKTPLQMLLRGQNLLGYRHYQDEVVDRFVQKSAENGIDVFRIFDALNDLRNLERSVQAVKQCGKHAQVAISYTISPIHTTAKFVEQAKRLVDMGCDSICIKDMAALIKPHATYDLVRGIKEACGDRIRIQLHAHATSGVTMVSYMKAVEAGVDGVDTAVSSMSLGPGHNPTESFAEMLENTGYTTRIDLGRVNKVKEHFAKVLPRYSEFLSTITGAETEIFRSQIPGGMLSNMESQLKQQGAGDRMRDVLEEIPLVRKDTGYVPLVTPTSQIVGTQAVLNVLMGRYKVLTGEFADLMLGYYGLTPGERNPEVVEQARRHANKEPIECRPADLLEPEWGKLRAAALPLEGCDGSDEDVLTYALFPQVAPKFFATRSEGPRNLGRDPVTGASETSIPEGHPGKITGPVTYTVTLSGQPHKVTVAPYGQE

SEQ ID NO: 63 - protein, 514 aa, Geobacter bemidjiensis
MSIEEKIKALNDKKSKLKLGGGRSKIDQQHAQGSLTARERIEALVDKDSFQEIGIFARHRCTNFGMAGKELPAEGVVTGAGSVGGRMVHLASQDFTVAGGSAGEVHSDKIVQAMLGSLKTGTPFVFMNDSGGARIQEGIDSLAGYGKVFYHNVMLSGVVPQISLICGPCAGGAAYSPALTDFIIQTAKARMFITGPSVIKEATGEEISAEELGGPLSQMNHSGVAHFVAENDLVALRICKKLLSYLPSNNIEDPPQLESDDVIVPDKTLNSIVPSEQKKAYDVRNVITRLIDGGDFLEVQPLFAANIVVGFGRILGRSVGIVANQPSVLAGALDINASDKGARFVRFCNAFNIPLVTLVDVPGFLPGVQQEKGGIIRHGAKMLFAYAAATVPKITVIMRKAYGGAFLAMCGKELETDRVFAWPSAEIAVMGPQGAVNVIFRNEIAQAEDPKKKRDELIASYQGTFATPYAAAARRDVDDIIEPADTRRHLAMTLDILSTKREFRPMKKHGLIPL

SEQ ID NO: 64 - protein, 448 aa, Pseudomonas putida
MNMPETGPAGIASQLKLDAHWMPYTANRNFQRDPRLIVAAEGNYLVDDHGRKIFDALSGLWTCGAGHTRKEIADAVTRQLSTLDYSPAFQFGHPLSFQLAEKIAELVPGNLNHVFYTNSGSECADTALKMVRAYWRLKGQATKTKIIGRARGYHGVNIAGTSLGGVNGNRKMFGQLLDVDHLPHTVLPVNAFSKGLPEEGGIALADEMLKLIELHDASNIAAVIVEPLAGSAGVLPPPKGYLKRLREICTQHNILLIFDEVITGFGRMGAMTGSEAFGVTPDLMCIAKQVTNGAIPMGAVIASSEIYQTFMNQPTPEYAVEFPHGYTYSAHPVACAAGLAALDLLQKENLVQSAAELAPHFEKLLHGVKGTKNIVDIRNYGLAGAIQIAARDGDAIVRPYEAAMKLWKAGFYVRFGGDTLQFGPTFNTKPQELDRLFDAVGETLNLID

SEQ ID NO: 65 - protein, 449 aa, Pseudomonas sp.
MNMPEHAAGSLASQLKLDAHWMPYTANRNFQRDPRLIVAAEGSWLVDDKGRKVYDSLSGLWTCGAGHTRKEIQEAVAKQLGTLDYSPGFQYGHPLSFQLAEKITSLTPGNLNHVFFTDSGSECADTAVKMVRAYWRLKGQATKTKMIGRARGYHGVNIAGTSLGGVNGNRKLFGQAMMDVDHLPHTLLASNAYSRGMPKEGGIALADELLKLIELHDASNIAAVFVEPMAGSAGVLVPPEGYLKRLREICDQHSILLVFDEVITGFGRTGSMFGADSFGVTPDLMCIAKQVTNGAIPMGAVIASSEIYQTFMNQPTPEYAVEFPHGYTYSAHPVACAAGLAALDLLQKENLVQSVAEIAPHFENALHGLKGAKNVIDIRNYGLAGAIQIAGRDGDAIVRPFEAGMALWKAGFYVRFGGDTLQFGPTFNSKPQDLDRLFDAVGEVLNKLD

SEQ ID NO: 66 - protein, 449 aa, Pseudomonas sp.
MNLPENAPTSLASQLKLDAHWMPYTANRNFQRDPRLIVGAEGSWLIDDKGRKVYDSLSGLWTCGAGHTRKEIQEAVARQLGTLDYSPGFQYGHPLSFQLAEKITDLTPGNLNHVFFTDSGSECADTAVKMVRAYWRLKGQSTKTKMIGRARGYHGVNIAGTSLGGVNGNRKLFGQAMMDVDHLPHTLLASNAYSRGMPEQGGIALADELLKLIELHDASNIAAVFVEPLAGSAGVLVPPQGYLKRLREICDQHNILLVFDEVITGFGRTGTMFGATTFGVTPDLMCIAKQVTNGAIPMGAVIASSEIYQTFMNQPTPEYAVEFPHGYTYSAHPVACAAGLAALDLLQKENLVQSVADVAPHFENALHGLKGTKNIIDIRNFGLAGAIQIAPRDGDAIVRPFEAGMALWKAGFYVRFGGDTLQFGPTFNSKPQDLDRLFDAVGEVLGKID

SEQ ID NO: 67 - protein, 449 aa, Pseudomonas sp.
MNMPENAPSPLASQLKLDAHWMPYTANRNFQRDPRLIVGAEGSWLIDDKGRKVYDSLSGLWTCGAGHTRKEIQEAVSKQLGTLDYSPGFQYGHPLSFQLAEKITDLTPGNLNHVFFTDSGSECADTAVKMVRAYWRLKGQSTKTKMIGRARGYHGVNIAGTSLGGVNGNRKLFGQAMMDVDHLPHTLLASNAFSRGMPEQGGIALADELLKLIELHDASNIAAVFVEPMAGSAGVLVPPQGYLKRLREICDQHNILLIFDEVITGFGRTGSMFGAESFGVTPDLMCIAKQVTNGAIPMGAVIASSEIYQTFMNQPTPEYAVEFPHGYTYSAHPVACAAGLAALDLLQKENLVQSVAEVAPHFENALHGLKGSKNVIDIRNYGLAGAIQIAARDGDAIVRPFEAGMALWKAGFYVRFGGDTLQFGPTFNSKPQDLDRLFDAVGEVLNKLD

SEQ ID NO: 68 - protein, 449 aa, Pseudomonas sp.
MNMPEHAAGLLASQLKLDAHWMPYTANRNFQRDPRLIVAAEGSWLVDDKGRKVYDSLSGLWTCGAGHTRKEIQEAVAKQLGTLDYSPGFQYGHPLSFQLAEKITSLTPGSLNHVFFTDSGSECADTAVKMVRAYWRLKGQATKTKMIGRARGYHGVNIAGTSLGGVSGNRKLFGQAMMDVDHLPHTLLASNAYSRGMPKEGGIALADELLKLIELHDASNIAAVFVEPMAGSAGVLVPPEGYLKRLREICDQHSILLVFDEVITGFGRTGSMFGADSFGVTPDLMCIAKQVTNGAIPMGAVIASSEIYQTFMNQPTPEYAVEFPHGYTYSAHPVACAAGLAALDLLQKENLVQSVAEIAPHFENALHGLKGAKNVIDIRNYGLAGAIQIAGRDGDAIVRPFEAGMALWKAGFYVRFGGDTLQFGPTFNSKPQDLDRLFDAVGEVLNKLD

SEQ ID NO: 69 - protein, 449 aa, Pseudomonas chlororaphis subsp. aureofaciens
MNMHENAPASLASQLKLDAHWMPYTANRNFQRDPRLIVAAEGSWLVDDKGRKVYDSLSGLWTCGAGHTRKEIQEAVAKQLGTLDYSPGFQYGHPLSFQLAEKITALTPGNLNHVFFTDSGSECADTAVKMVRAYWRLKGQSTKTKMIGRARGYHGVNIAGTSLGGVNGNRKLFGQAMMDVDHLPHTLLASNAFSRGIPEQGGIALADELLKLIELHDASNIAAVFVEPLAGSAGVLVPPQGYLKRLREICDQHNILLVFDEVITGFGRTGKMFGADSFGVTPDLMCIAKQVTNGAIPMGAVIASSEIYQTFMNQPTPEYAVEFPHGYTYSAHPVACAAGLAALDLLQKENLVQSVADVAPHFEKALHGIKGAKNVIDIRNFGLAGAIQIAPRDGDAIVRPFEAGMALWKAGFYVRFGGDTLQFGPTFNSKPQDLDRLFDAVGEVLNKLD

SEQ ID NO: 70 - protein, 449 aa, Pseudomonas moorei
MNLPENAPTSLASQLKLDAHWMPYTANRNFQRDPRLIVGAEGSWLIDDKGRKVYDSLSGLWTCGAGHTRKEIQEAVAKQLGTLDYSPGFQYGHPLSFQLAEKITDLTPGNLNHVFFTDSGSECADTAVKMVRAYWRLKGQSTKTKMIGRARGYHGVNIAGTSLGGVNGNRKLFGQAMMDVDHLPHTLLASNAFSRGMPEQGGIALADELLKLIELHDASNIAAVFVEPMAGSAGVLVPPQGYLKRLREICDQHSILLVFDEVITGFGRTGNMFGADTFGVTPDLMCIAKQVTNGAIPMGAVIASSEIYQTFMNQPTPEYAVEFPHGYTYSAHPVACAAGLAALDLLQKENLVQSVAEVAPHFENALHGLKGTKNIIDIRNFGLAGAIQIAPRDGDAIVRPFEAGIALWKAGFYVRFGGDTLQFGPTFNSKPQDLDRLFDAVGEVLSKID

SEQ ID NO: 71 - protein, 449 aa, Pseudomonas synxantha
MNMPENAPSTLASQLKLDAHWMPYTANRNFQRDPRLIVAAEGSWLTDDKGRKVYDSLSGLWTCGAGHTRKEIQEAVAKQLGTLDYSPGFQYGHPLSFQLAEKITDLTPGNLNHVFFTDSGSECADTAVKMVRAYWRLKGQATKTKMIGRARGYHGVNIAGTSLGGVNGNRKLFGQAMMDVDHLPHTLLTSNAFSRGMPEQGGIALADELLKLIELHDASNIAAVFVEPMAGSAGVLVPPQGYLKRLREICDQHNILLVFDEVITGFGRTGSMFGADSFGVTPDLMCIAKQVTNGAIPMGAVIASSEIYQTFMNQATPEYAVEFPHGYTYSAHPVACAAGLAALDLLQKENLVQSVAEVAPHFENALHGLKGSKNVIDIRNYGLAGAIQITPRDGDAIVRPFEAGMALWKAGFYVRFGGDTLQFGPTFNSKPQDLDRLFDAVGEVLNKID

SEQ ID NO: 72 - protein, 449 aa, Pseudomonas chlororaphis subsp. aurantiaca
MNMHENAPASLASQLKLDAHWMPYTANRNFQRDPRLIVAAEGSWLVDDKGRKVYDSLSGLWTCGAGHTRKEIQEAVAKQLGTLDYSPGFQYGHPLSFQLAEKITALTPGNLNHVFFTDSGSECADTAVKMVRAYWRLKGQSTKTKMIGRARGYHGVNIAGTSLGGVNGNRKLFGQAMMDVDHLPHTLLASNAFSRGIPEQGGIALADELLKLIELHDASNIAAVFVEPLAGSAGVLVPPQGYLKRLREICDQHNILLVFDEVITGFGRTGKMFGADSFGVTPDLMCIAKQVTNGAIPMGAVIASSEIYQTFMNQPTPEYAVEFPHGYTYSAHPVACAAGLAALDLLQKENLVQSVAEVAPHFEKALHGIKGAKNVIDIRNFGLAGAIQIAPRDGDAIVRPFEAGMALWKAGFYVRFGGDTLQFGPTFNSKPQDLDRLFDAVGEVLNKLD

SEQ ID NO: 73 - protein, 449 aa, Pseudomonas asplenii
MNMPENAPSSLASQLKLDAHWMPYTANRNFQRDPRLIVGAEGSWLIDDKGRRVYDSLSGLWTCGAGHTRKEIQEAVARQLGTLDYSPGFQYGHPLSFQLAEKITALTPGNLNHVFFTDSGSECADTAVKMVRAYWRLKGQPTKTKMIGRARGYHGVNIAGTSLGGVSGNRKLFGQAMHDVDHLPHTLLASNAFSRGMPEQGGIALAEELLKLIELHDASNIAAVFVEPMAGSAGVLVPPQGYLKRLREICDQHSILLVFDEVITGFGRTGAMFGADSFGVTPDLMCIAKQVTNGAIPMGAVIASSEIYQTFMNQPTPEYAVEFPHGYTYSAHPVACAAGLAALDLLQKESLVQSVAEVAPHFENVLHGLKGSKNVIDIRNFGLAGAIQIAPRDGDAIVRPFEAGMALWKAGFYVRFGGDTLQFGPTFNSKPQDLDRLFDAVGEVLSKLD

SEQ ID NO: 74 - protein, 450 aa, Lactobacillus brevis
MKVTVVGCTHAGTFAIKQILAEHPDAEVTVYERNDVISFLSCGIALYLGGKVADPQGLFYSSPEELQKLGANVQMNHNVLAIDPDQKTVTVEDLTNHAQTTESYDKLVMTSGSWPIVPKIPGIDSDRVKLCKNWAHAQALIEDAKEAKRITVIGAGYIAAELAEAYSTTGHDVTLIARSARVMRKYFDADFTDVIEQDYRDHGVQLALGETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGLNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNA

SEQ ID NO: 75 - protein, 451 aa, Lactobacillus
MKVIVVGSSHGGYETVRGILAAQPDTEIQWYEKGNFLSFLSCGMQLYLEGAVKDVNSVSYATPAGMQAQGVHVFVNSEISKVDPASHSVHVIDHATGDERDEAYDKLVLSVGAVPFDLPVPGHDLANIYAMRGRDWAIKLKAKTVDPSVKNVVVIGSGYIGIEAAEVFAKAGMHVTVIDLLPRLLSLYLDQEFTDILTKTMADHGIYAAVGQGIKAYEGVDGHVTKVVTDQGEYPADLVVTAAGIRPATGFLKGVVDLDDHGLIKINDHLQTSDTDIYAVGDATLVPFAPTGKDNRIALATNARRQGRIAAKNLLGENVPMPAVSGSSALSVFDYHFASTGVKEGTADKLGVKTASVLVTDTIRPKFVSEDAGNTKVWFKLTFDPTDGRVLGAQIMSKYDVTANINAISVAIQAKLTVADLAYTDFFFQPGFDRPWNIINVAAQKAAATLN

SEQ ID NO: 76 - protein, 438 aa, Lactobacillus
MAKIIIVGAAHGGRETVNGLLAANTDNEIHWYEHGQFATALDWAPADAEKERLALSQQVTLFDQTTVTRITPATHTITARNQRGQLQTDHYDRLVLSVGSLPIQLPIPGAELSGVRSIQNRASINELKLAAKSAAIKNVVVIGGGYIGMNFAALFKQTGKQVTVIDVNARPFSHNLDSEFTQILAAASVENGLQLKMEERVTAVLGSTHVTAVQTNRGQYAADLVLVAVGNRPNTAWLRGTLALDSEGLIETDDYFQTSVPDIYAIGDATKVRFTPTGTKERITLGSAASHAGRLLAHNLLTDQRIVFPGVQATSALNAAGYYFAATGLNTQLAVRMQQPVLATYIAVPRLVASAPARLNATVHFKLFYDKTHRILGAQIMSTAELTAVINTVSLAIQMGATLEQLAYGDFFFQPGLSQPVDVLSTASLQALQTLATN

SEQ ID NO: 77 - protein, 450 aa, Lactobacillus
MKVIVIGCTHAGTAAVNQILASNPDTEVTIYERNDNVSFLSCGIALYLGGQVADPQGLFYSSPEQLAKLGATVHMQHDVTDVNTDKHEITVTDLKTGESKTDHYDKLVVTTGSWPVIPPIDGIDSPNVYLCKNWTHAQNLWEAAKPAKRVIVIGGGYIGTELVEAYQKQGKEVTLIDGLPRILNKYLDKEFTDRVEQDFVDHGIKMALNQMVQGFSDDGKEVTVKTDKGSYTADMAILCVGFRPNTGLLKGKVDMNANGSIKTNDYMQTSDPDIYGAGDSVAVHYNPTKKDAYIPLATNAVRQGTLVGLNIFKPTRKYMGTQSTSGLMLFGQTIVSSGMTLEHAQAENVPAAAVTFEDNYRPEFMPTTKPVLMQLVYNPETREILGAQFMSEHDVSQSANVISVMIQNHNTIDDLGFVDMFFQPIYDRPFNYLNLLGQAAIAHAAEAVTE

SEQ ID NO: 78 - protein, 449 aa, Lactobacillus reuteri
MKVIIVGCTHAGTITATQILQNHPETEVTIYERNDNVSFLSCGIAVYLSGDVGNPDAMFYSSPEQLAAMGATVHMQHNVTDIDPKTKTVTVTDLVTGETKTDHYDKLVDTTGSWPVIPPIEGVDGPHVYLCKNYHHAKELFNVAKDAQRIVVIGGGYIGVELVEAYTRQNKDVTLIDGSPRMLHKYFDREYTDRIQQEFVDHGAHFAFDQRVTGFENHENGVTVKTDKGNYEADIAILCVGFRPNTDLLKGKVKMHDNGAIITNEYMQSSDPDIYAAGDSTAVHYNPTGKDAYIPLATNAIRQGTIVGTNLFGNTMRDMGTQSSSGLNLYGTTMVSSGLTLENAKEAGFDAAAVTVEDNYRPEFMPTTTPVLMTLVWDKKTRQILGGQFMSKHDVSQSANIISLCIQDKHTIDYLAFVDMLFQPHFDRPFNYVNILGQAAVKKQAELEK

SEQ ID NO: 79 - protein, 447 aa, Lactobacillus salivarius
MTKTVIIGTNHAGIAAANTLLDNYPDQEVVMIDRNSNLSYLGCGTALWVGRQVNDYHGLFYTKVEDFTNKGAKITLEAEVKDIDFDKKVVHYIKKDGTELTEDYDKLILATGSLPISPNVAGKDLNGIHFLKLFQDGQDVDKEISAEEVKNVAVIGAGYIGVEIAEAAKRRGKNVLLFDAADRSLSTHYDKAFTDLMDKNLADHGIENHFGELVTEYKGNDQNHVTQVVTAEGEYDVDLVINAIGFRPNNSLGKDHLKLFKNGAYLVDRYGQTSDKNVYAVGDCATIYSNALDKMTYIALASNAVRSGIVAAHNLAGTKLESVGVQGSNGINIWDLKMVSTGLSVEAAKKFGLDVEYTDFEDLQKPAFMPKDVNAEVKIRIVYEKESRRIVGAQLASTEDVSMAIHMFSLAIQEHVTIDKLSLLDIFFLPHFNQPYNYITMAALSAK

SEQ ID NO: 80 - protein, 450 aa, Lactobacillus lindneri
MKVIVVVCTHAGTFAIKRAIADHPDAEVTVYEKNDNISFLSCGIALYLGKEIKNNDPQGLFYSSPEELESLGANVQMKHEVTEIDSKAKTIKVKDLTSGEEKEETYDKLIMTTGSLPIVPPIPGIDSKRVYLCKNWNDAKKLFDEAPKAKSITVIGSGYIGAELAEAYSNQNFDVNLVDGSERVLYKYFDPKFTEILAEDYENHGVNLELGSKVAAFEEQADGIIVKTNDDKEIKSDIAILCIGFKPNTSLLDGQVDMMDNGAIITDDYMHSTNHDIFAAGDSAAVHYNPTGQNAYIPLATNAVRQGLLVGENLTEDKTKYMGTQSSSGLKLYGRTYVSTGLNMAAAKAQNINAHQVIVEDNYRPEFMKTTDSVLMSLVYDPDSRRILGGALTSMYDVSQSANVLSVCIQNKNTIDDLAMVDMLFQPQFDRPFNYLNILAQAAQAKETKK

SEQ ID NO: 81 - protein, 453 aa, Lactobacillus casei subsp. casei ATCC 393
MLMKVVVVGCTHAGTAAVRELLTRHPETEVDVFERREDISFLSCGIALYLEGTVGRLEDMFYATPASLEALGPKVHVHLKHDVLSIDAVDHQVVAEDLATGQQQHYQYDKLIMTTGSYPVIPPISGVSIPRVLMCKSYDDARKIKESAKDADKIAIVGGGYIGVELAEAYSRNYKQVILINGVSPLLSHYVDLPLSKEIASVLTKRGVELKTNTIARHFDSDAKHVFIQTDQGEIQADLAVVCVGFRPMTELLVGQVEMNPDGAIHVNDYMQTSNPDIYAAGDAVAVHFNPTGKDAYAPLATNAVRQGKMAGANIFGQNIKYMGTQATSALKLYDHSLAVTGLTLAHAKRNHLPAASVTMTDDFRPPFMPHTVKITMVLVYDTRDRRILGAQFYSQYDIANAANLISVMIQNRNTIDQLAYVDMLFNPNYDKPWHYLNLLGQLAVMQADNAKL

SEQ ID NO: 82 - protein, 444 aa, Lactobacillus sakei
MKVAVIGCTHAGTFATQATLTAHPDWDVTVYETHDNLSFLSCGIALWVGGHVSDPKKMFYSSPEMLAQLGAHMKMQHTVLKADLKEKVLHVKNLVTSEETIEKFDKIIVTTGSSPVIPPIEGIDNPNVKLCKNWEHANELKKSAPDVKSVIVIGSGYIGAELAEAYATTDKKVTLIDALPHALGKNFDPNISEIVEQDYRDHGVQLALSEKVVSFSGTDQVTVKTDKNEYTADLAILCVGFRPNTGLFKDQVEMLGNGAITTDEYMQTSVKGVYAAGDSAAVYYNPTQKADYIPLATNAVRQGLLIGRNIETPTEKYMGTQATSAVELFGRTYSGSGLTVEGAKVRGMAVATTTLTEDYRPDFMPSTTPVLMNLTWNPKTRQVLGGSFTSQYDIAQAANVISMAIQTKMTIDQLAMVDMFFQPNYDQPLNYVNAIAMKAVAESH

SEQ ID NO: 83 - protein, 449 aa, Lactobacillus backii
MYMKVVIVGCTHAGTAAAKQILASSPNTEVTVYERHQDISFLSCGISLYLSGKVKSLEEMFYATPEDLEQLGAKVRTNCDVYAIDTKHKTLRVEELQTGIKYQDTYDKLVMATGSVPVVPPIEGVSLPGILMCKDYEDAKQIYQTAKSAKRIAIIGGGYVGVELVEAYSRTGHDVMLINGLHRPLSHYVDVPFAKEVEADMKQHGVQLYLNEVAHTFDSDDQHIYIKTNKAEREADLAVVCVGFRPQTDLLEGQVELTKDGAIHVNDYMQTSDPDIFAAGDSVAVHFNPTQTDAYTPLATNAVRQGALVGRNIFKPTLKYLGTQATSALHLYGKTVASTGLTLERAQKHGFTAAAVDLTDNYRPEFMESTTPVEMRLVYDQNDHRILGGQLVSKYDIAQSANLLSVCIQNKNTIEDLAFVDMLFSPYDDRPFNYLNLLGQAAMQQEEAR

SEQ ID NO: 84 - protein, 452 aa, Lactobacillus buchneri
MKVAVIGCTHAGTFAIKTILAEHPDAEVTVYEKNDNISFLSCGIALYLGGHVKDPQGLFYSSPDELKKLGASVKMNHEVTNVDPASKTIEVTDLKNDQSFEDTYDKLVVTTGSWPVIPPIKGVDNKNVYLCKNWNHAKELFETAPEKKRITIVGAGYIGAELAEAYNTKGHEVTLLDGADRIMSKYFDKEFTDVVEKDFTDHGVTLGLDQQVTEISGDDTVTIKTTKGTYETDLVVLCVGFRPNTGLFKGKLAMTDNGTLITNEYMQTSDPDIYGAGDSVAVHYNPTHGLAYIPLATNAVRQGILVGKNIEKPTVKYMGTQSSSGLKLYDKTIVSTGLTMAAAKAQNINASEVIITDNYRPEFMPTTEPVLMSLVYDPETRRILGGSLLSTYDVSQSANTLSVCIQNENTIDDLAMVDMLFQPQFDRPFNYLNILAQAAQEQAAKLTKATSK

SEQ ID NO: 85 - protein, 446 aa, Lactobacillus antri
MKIVIIGCTHAGTITASQVLSQHPDAEVTIYEKNDNVSFLSCGIATYLGGVAKNIDDMFYSSPEALSQLGAAVHMQHEVTAVDLQKKTLQVKDLVSGEETEDHYDKLMVTTGSWPVIPNLPGIDDPNVYLCKNYNHAQKLFEVAQNAQKVVVIGGGYIGVELVEAYSKQGKQVTLIDGNDRLLSKYYDAKYTDIVTKDFEDHQVTLALGELVAGFEPADQGVVVKTDRGSYDADIAILCIGFRPNTSLFKDQLEFNADGSIKTNAYMQTSDPDVYGAGDSVAVHYNPTNQEAYVPLATNAVRQGIIAGTNLYGNTMTYMGTQSTSGLQLYDNAMSSTGMTLESAKAAGFDADAVTFEDHYRPEFMPTNATVIGTVVWDRSNNQILGAQFMSKLDISMYANAVSVMIQNHNTIDFMAMVDMLFQPNFDRPFNFINLLGQQAVAKAVK

SEQ ID NO: 86 - protein, 448 aa, Lactobacillus iners
MKFVIVGTNHAGIAAANTLLDNYGKNNEVIMIDRNNNLSYLGCGTALWVGRQISSYKKLFYTKPADFEAKGARISLETTVNKIDFENKIVYATIKNHTEITETYDKLILAEGSAPIAPDLPGKDLAGIHFLKLFQEGMAVDQELDKSDVKTVAVVGAGYIGVEIAEAARRRGKQVLLFDAAKTCLSSYYDPEFSTLMDQNLRDHDIETHYGELAQEYLGKDGHVCALKTNKGKYQADIVINCIGFRPNAILGGDHLKRFSNGAYLVNRNFQTSDPDVFAVGDCATNYSNALQDTTYIALASNAVRSGIVGAHNAAGTVIEGAGVQGSNGIEIYDLKLVSTGLSVKAADKFGLSVKYVDYEDKQLPDFMPASENAKVKIRIVYEKKTNRIVGAQLASLYDMHELIHMFSLAIQEGLTMDKLQLLDIFFLPHYNQPYNYVTMAALKADKN

SEQ ID NO: 87 - protein, 543 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 88 - protein, 579 aa, Streptomyces sp. SN-593
MELALPAELAPTLPEALRLRSEQQPDTVAYVFLRDGETPEETLTYGRLDRAARARAAALEAAGLAGGTAVLLYPSGLEFVAALLGCMYAGTAGAPVQVPTRRRGMERARRIADDAGAKTILTTTAVKREVEEHFADLLTGLTVIDTESLPDVPDDAPAVRLPGPDDVALLQYTSGSTGDPKGVEVTHANFRANVAETVELWPVRSDGTVVNWLPLFHDMGLMFGVVMPLFTGVPAYLMAPQSFIRRPARWLEAISRFRGTHAAAPSFAYELCVRSVADTGLPAGLDLSSWRVAVNGAEPVRWTAVADFTEAYAPAGFRPQAMCPGYGLAENTLKLSGSPEDRPPTLLRADAAALQDGRVVPLTGPGTDGVRLVGSGVTVPSSRVAVVDPGTGTEQPAGRVGEIWINGPCVARGYHGRPAESAESFGARIAGQEARGTWLRTGDLGFLHDGEVFVAGRLKDVVIHQGRNFYPQDIELSAEVSDRALHPNCAAAFALDDGRTERLVLLVEADGRALRNGGADALRARVHDAVWDRQRLRIDEIVLLRRGALPKTSSGKVQRRLARSRYLDGEFGPAPAREA

SEQ ID NO: 89 - protein, 744 aa, Saccharomyces cerevisiae Kyokai no. 7
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDAKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCIDGLQGFEKLHNIKVGLEPLTLEDDVVTPTFKIKRAKASKFFKDTLDQLYAEGSLVKTEKL

SEQ ID NO: 90 - protein, 700 aa, Saccharomyces cerevisiae S288C
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 91 - protein, 694 aa, Saccharomyces cerevisiae S288C
MSEQHSVAVGKAANEHETAPRRNVRVKKRPLIRPLNSSASTLYEFALECFNKGGKRDGMAWRDVIEIHETKKTIVRKVDGKDKSIEKTWLYYEMSPYKMMTYQELIWVMHDMGRGLAKIGIKPNGEHKFHIFASTSHKWMKIFLGCISQGIPVVTAYDTLGESGLIHSMVETESAAIFTDNQLLAKMIVPLQSAKDIKFLIHNEPIDPNDRRQNGKLYKAAKDAINKIREVRPDIKIYSFEEVVKIGKKSKDEVKLHPPEPKDLACIMYTSGSISAPKGVVLTHYNIVSGIAGVGHNVFGWIGSTDRVLSFLPLAHIFELVFEFEAFYWNGILGYGSVKTLTNTSTRNCKGDLVEFKPTIMIGVAAVWETVRKAILEKISDLTPVLQKIFWSAYSMKEKSVPCTGFLSRMVFKKVRQATGGHLKYIMNGGSAISIDAQKFFSIVLCPMIIGYGLTETVANACVLEPDHFEYGIVGDLVGSVTAKLVDVKDLGYYAKNNQGELLLKGAPVCSEYYKNPIETAVSFTYDGWFRTGDIVEWTPKGQLKIIDRRKNLVKTLNGEYIALEKLESVYRSNSYVKNICVYADESRVKPVGIVVPNPGPLSKFAVKLRIMKKGEDIENYIHDKALRNAVFKEMIATAKSQGLVGIELLCGIVFFDEEWTPENGFVTSAQKLKRREILAAVKSEVERVYKENS

SEQ ID NO: 92 - protein, 694 aa, Saccharomyces cerevisiae CEN.PK113-7D
MTEQYSVAVGEADNEHETAPRRNIRVKDKPLIRPINSSASTLYEFALECFTKGGKRDGMAWRDIIDIHETKKTIVKRVDGKDKPIEKTWLYYELTPYITMTYEEMICVMHDIGRGLIKIGVKPNGENKFHIFASTSHKWMKTFLGCMSQGIPVVTAYDTLGESGLIHSMVETDSVAIFTDNQLLSKLAVPLKTAKNVKFVIHNEPIDPSDKRQNGKLYKAAKDAVDKIKEVRPDIKIYSFDEIIEIGKKAKDEVELHFPKPEDPACIMYTSGSTGTPKGVVLTHYNIVAGIGGVGHNVIGWIGPTDRIIAFLPLAHIFELIFEFEAFYWNGILGYATVKTLTPTSTRNCQGDLMEFKPTVMVGVAAVWETVRKGILAKINELPGWSQTLFWTVYALKERNIPCSGLLSGLIFKRIREATGGNLRFILNGGSAISIDAQKFLSNLLCPMLIGYGLTEGVANACVLEPEHFDYGIAGDLVGTITAKLVDVEDLGYFAKNNQGELLLKGAPICSEYYKNPEETAAAFTDDGWFRTGDIAEWTPKGQIKIIDRKKNLVKTLNGEYIALEKLESIYRSNPYVQNICVYADENKVKPVGIVVPNLGHLSKLAIELGIMVPGEDVESYIHEKKLQDAVCKDMLSTAKSQGLNGIELLCGIVFFEEEWTPENGLVTSAQKLKRRDILAAVKPDVERVYKENT

SEQ ID NO: 93 - protein, 549 aa, Pseudomonas aeruginosa WS136
MMVPTLEHELAPNEANHVPLSPLSFLKRAAQVYPQRDAVVYGARRYSYRQLHERSCALASALERVGVQAGERVAILAPNIPEMLEAHYGVPGAGAVLVCINIRLEARSIAFILRHCAARVLICDREFGAVAQQALAMLDAPPLLVGIDDDQAERAGLAHDLDYEAFLAQGDPGRPLSAPQNEWQSIAINYTSGTTGDPKGVVLHHRGAYLNACAGALIFQLGPRSVYLWTLPMFHCNGWSHTWAVTLSGGTHVCLRKVQPDAIHAAIAEHAVTHLSAAPVVMSMLIHAEHASVPAVPVSVITGGAAPPSAVIAAMEARGFSITHAYGMTESYGPSTLCLWQPGVDELPLEARAQFMSRQGVAHPMLEEATVLDTDTGRPVPADGRTLGELVVRGNTVMKGYLHNPEATRAALADGWLHTGDLAVLHPDGYVEIKDRAKDIIISGGENISSLEIEEVLYQHPEVVEAAVVARPDSRWGETPHAFVALRADALPRTTGDDLVRWCRERLAHFKAPRHVTLTELPKTATGKIQKFVLREWARQEEEAGDAGR

SEQ ID NO: 94 - protein, 576 aa, Streptomyces albus
MSRTAASAEGHDTGVREFLAARDFLLAHREDYERAYTEFRWPVLDEFNWALDWFDRLGGTPESASRTALWIVEEDGSERKWSFGELSARSGQVANWLREQGVERGDRIVLMLGNQVELWETILAAMKLGAVVIPATTLLGEADLRDRLTRGEARHVVVASSEAGKWDAVPGEYTRIAAGAPVDGWLSYAESRSAPVDFVPDAVTRAADTLLLYFTSGTTSKPKLVEHTHVSYPVGHLSTMYWIGLEPGDVHLNISSPGWAKHAWSNVYAPWNAGATVLIYNYTRFDATSMLEQLGRCGVTSFCAPPTVWRMLIQAELGSLKTPPTKVVGAGEPLNPEVIDQVERAWGVRIRDGFGQTETTVQIGNSPGQPVKPGSMGRPMPGYRVALVDTATGEPGDEGEICLELAARPLGLMAGYREESGRNTEATSGEHYHTGDVAARDAEGYITYVGRADDVFKASDYRISPFELESTLIQHPAVAEAAVVPAPDPVRLAVPKAYVVLTAGHRPDRETALSIIRYAREQLAPYKRIRRLEFTDLPKTISGKIRRVQLRAQEDNTPREQEYREEDFPELRSRGT

SEQ ID NO: 95 - protein, 577 aa, Homo sapiens
MQWLMRFRTLWGIHKSFHNIHPAPSQLRCRSLSEFGAPRWNDYEVPEEFNFASYVLDYWAQKEKEGKRGPNPAFWWVNGQGDEVKWSFREMGDLTRRVANVFTQTCGLQQGDHLALMLPRVPEWWLVAVGCMRTGIIFIPATILLKAKDILYRLQLSKAKGIVTIDALASEVDSIASQCPSLKTKLLVSDHSREGWLDFRSLVKSASPEHTCVKSKTLDPMVIFFTSGTTGFPKMAKHSHGLALQPSFPGSRKLRSLKTSDVSWCLSDSGWIVATIWTLVEPWTAGCTVFIHHLPQFDTKVIIQTLLKYPINHFWGVSSIYRMILQQDFTSIRFPALEHCYTGGEVVLPKDQEEWKRRTGLLLYENYGQSETGLICATYWGMKIKPGFMGKATPPYDVQVIDDKGSILPPNTEGNIGIRIKPVRPVSLFMCYEGDPEKTAKVECGDFYNTGDRGKMDEEGYICFLGRSDDIINASGYRIGPAEVESALVEHPAVAESAVVGSPDPIRGEVVKAFIVLTPQFLSHDKDQLTKELQQHVKSVTAPYKYPRKVEFVSELPKTITGKIERKELRKKETGQM

SEQ ID NO: 96 - protein, 651 aa, Streptomyces coelicolor
MSNESLANLLKEERRFAPPADLAANANVTAEAYEQAKADRLGFWAEQARRLTWAKEPTETLDWSNPPFAKWFKDGTLNVAYNCVDRHVEAGNGDRVAIHFEGESGDSRALTYAQLKDEVSKAANALLELGVQKGDRVAIYMPMIPETAIAMLACARIGAAHSVVFGGFSSDALATRIQDADARVVITADGGYRRGKPSALKPAVDEAVERAGIVEHVLVVRRTGQDVAWDDSRDKWWHETVDGQSAEHTPEAFDAEHPLFILYTSGTTGKPKGILHTSGGYLTQTAYTHWAVFDLKPETDVFWCTADVGWVTGHSYIVYGPLANGATQVMYEGTPDTPHQGRFWEIVQKYGVTILYTAPTAIRTFMKWGDDIPAKFDLSSLRVLGSVGEPINPEAWIWYRKNIGADATPVVDTWWQTETGAMMITPLPGVTHAKPGSAQRPLPGISATVVDDEANEVPNGGGGYLVLTEPWPSMLRTIWGDDQRFIDTYWSRFEGKYFAGDGAKKDDDGDIWLLGRVDDVMLVSGHNISTTEVESALVSHPSVAEAAVVGATDETTGQAIVAFVILRGTTAESEDLVAELRNHVGATLGPIAKPKRILPVSELPKTRSGKIMRRLLRDVAENRQVGDVTTLADSTVMDLIQTKLPAAPSED

SEQ ID NO: 97 - protein, 250 aa, Bacillus sp.
MTVKKLYFVPAGRCMLDHSSVNSTLTPGELLDLPVWCYLLETEEGPILVDTGMPESAVNNEGLFNGTFVEGQVLPKMTEEDRIVNILKRVGYEPEDLLYIISSHLHFDHAGGNGAFINTPIIVQRAEYEAAQHSEEYLKECILPNLNYKIIEGDYEVVPGVQLLHTPGHTPGHQSLLIETEKSGPVLLTIDASYTKENFENEVPFAGFDSELALSSIKRLKEVVMKEKPIVFFGHDIEQERGCKVFPEYI

SEQ ID NO: 98 - protein, 263 aa, Rhizobium radiobacter
MTDIRLYMLQSGTLKCKVHNIKMNQGNGADYEIPVPFFLITHPGGHTVIDGGNAIEVATDPRGHWGGICDVYWPVLDKDQGCVDQIKALGFDPADVKYVVQSHLHLDHTGAIGRFPNATHIVQRSEYEYAFTPDWFAGGGYIRKDFDKPGLKWQFLNGTQDDYYDVYGDGTLTTIFTPGHAPGHQSLLVRLPNSKPLLLTIDAAYTLDHWEEKALPGFLASTVDTVRSVQKLRTYAEKHDATVVTGHDPDAWANFKKAPEFYA

SEQ ID NO: 99 - protein, 276 aa, Agrobacterium genomosp. 1 str. S56
MGNKLFVLDLGEIRVDENFIIANSTFVTPQKPTVSSRLIDIPVSAYLIQCTDATVLYDTGCHPECMGTNGRWPAQSQLNAPYIGASECNLPERLRQLGLSPDDISTVVLSHLHNDHAGCVEYFGKSRLIAHEDEFATAVRYFATGDHSSPYIVKDIEAWFAAPRNWDLVSRDERERELAPGVTLLNFGSGHAAGMLGIAVELEKRPGFLLVSDACYTATNYGPPARRAGVLHDTIGYDRTVSYIRKYAESRSLKVLFGHDREQFASLIKSTDGFYE

SEQ ID NO: 100 - protein, 280 aa, Ralstonia solanacearum
MLKFPRIAARLALASVVILAAGCSSLPARQTSSVERLYILNCGEGVAADISRWSPGVNVGKPMAFADNCYLIKHAQGWLLWDTGVDDAIAALPDGQAPADPKAIHWRRPKTLASQLAQLGVKPADIRYVAISHTHPDHIGNVEMFPQAMLLVQKAEYEWPGADHAPRFKPGHPVTEIEGDKDVFGDGSVMLISTPGHTPGHQSLLVKLPKTGALVLSGDAIHFQSNWDNRRVPSINFDKDKTLASMQRIADLLVREKAQLWINHDKAQRDTLKMAPAYYE

SEQ ID NO: 101 - protein, 283 aa, Escherichia coli (strain K12)
MRTQWPSPAKLNLFLYITGQRADGYHTLQTLFQFLDYGDTISIELRDDGDIRLLTPVEGVEHEDNLIVRAARLLMKTAADSGRLPTGSGANISIDKRLPMGGGLGGGSSNAATVLVALNHLWQCGLSMDELAEMGLTLGADVPVFVRGHAAFAEGVGEILTPVDPPEKWYLVAHPGVSIPTPVIFKDPELPRNTPKRSIETLLKCEFSNDCEVIARKRFREVDAVLSWLLEYAPSRLTGTGACVFAEFDTESEARQVLEQAPEWLNGFVAKGANLSPLHRAML

SEQ ID NO: 102 - protein, 245 aa, Thermoplasma acidophilum
MMILKIGGSVITDKSAYRTARTYAIRSIVKVLSGIEDLVCVVHGGGSFGHIKAMEFGLPGPKNPRSSIGYSIVHRDMENLDLMVIDAMIEMGMRPISVPISALRYDGRFDYTPLIRYIDAGFVPVSYGDVYIKDEHSYGIYSGDDIMADMAELLKPDVAVFLTDVDGIYSKDPKRNPDAVLLRDIDTNITFDRVQNDVTGGIGKKFESMVKMKSSVKNGVYLINGNHPERIGDIGKESFIGTVIR

SEQ ID NO: 103 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 104 - protein, 371 aa, Corynebacterium glutamicum
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRM

SEQ ID NO: 105 - protein, 381 aa, Abies grandis
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 106 - protein, 307 aa, Arabidopsis thaliana
MATTSTTTKLSVLCCSFISSPLVDSPPSLAFFSPIPRFLTVRIATSFRSSSRFPATKIRKSSLAAVMFPENSVLSDVCAFGVTSIVAFSCLGFWGEIGKRGIFDQKLIRKLVHINIGLVFMLCWPLFSSGIQGALFASLVPGLNIVRMLLLGLGVYHDEGTIKSMSRHGDRRELLKGPLYYVLSITSACIYYWKSSPIAIAVICNLCAGDGMADIVGRRFGTEKLPYNKNKSFAGSIGMATAGFLASVAYMYYFASFGYIEDSGGMILRFLVISIASALVESLPISTDIDDNLTISLTSALAGFLLF

SEQ ID NO: 107 - protein, 450 aa, Lactobacillus brevis
MKVTVVGCTHAGTFAIKQILAEHPDAEVTVYERNDVISFLSCGIALYLGGKVADPQGLFYSSPEELQKLGANVQMNHNVLAIDPDQKTVTVEDLTSHAQTTESYDKLVMTSGSWPIVPKIPGIDSDRVKLCKNWAHAQALIEDAKEAKRITVIGAGYIGAELAEAYSTTGHDVTLIDAMDRVMPKYFDADFTDVIEQDYRDHGVQLALSETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGVNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNA

SEQ ID NO: 108 - protein, 182 aa, Escherichia coli (strain K12)
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

(19) United States
(12) Patent Application Publication
(10) Pub. No.: US 2022/0127649 A1
(43) Pub. Date: Apr. 28, 2022

(54) ENGINEERED CELLS FOR IMPROVED PRODUCTION OF CANNABINOIDS

(71) Applicant: Genomatica, Inc., San Diego, CA (US)

(72) Inventors: Michael A. Noble, San Diego, CA (US); Jonathan Moore, San Diego, CA (US); Jingyi Li, San Diego, CA (US); Anna Lechner, San Diego, CA (US)

(21) Appl. No.: 17/427,019

(22) PCT Filed: Jan. 30, 2020
(86) PCT No.: PCT/US2020/015913

§ 371 Date: Jul. 29, 2021

Related U.S. Application Data
(60) Provisional application No. 62/802,085, filed on Feb. 6, 2019.
(60) Provisional application No. 62/798,926, filed on Jan. 30, 2019.

(51) Int. Cl. C12P 7/42; C12N 1/20; C12N 15/52; C12N 15/70; C12P 7/22

(52) U.S. Cl. CPC C12P 7/42; C12N 1/20; C12N 2800/101; C12N 15/70; C12P 7/22; C12N 15/52

(57) ABSTRACT

The invention provides non-natural microbial organisms containing enzymatic pathways and/or metabolic modifications for enhancing synthesis of olivetolic acid, olivetolic acid derivatives and/or cannabinoids.

### PRIORITY CLAIM

This application claims the benefit of U.S. Provisional Patent Application Ser. No. 62/798,926, filed Jan. 30, 2019, and 62/802,085 filed Feb. 6, 2019, both entitled ENGINEERED CELLS FOR IMPROVED PRODUCTION OF CANNABINOIDS, wherein said applications are incorporated herein by reference in their entireties. Also, the entire contents of the ASCII text file entitled “GNO0105WO_Sequence_Listing.txt” created on Jan. 30, 2020, having a size of 425 kilobytes is incorporated herein by reference.

### FIELD OF THE DISCLOSURE

The present disclosure relates to the engineering of cells and microorganisms for the production of cannabinoids, to cultures of such cells and microorganisms, and to methods of making cannabinoids using the cultures and cells provided herein.

### BACKGROUND OF THE DISCLOSURE

Cannabinoids are prenylated isoprenoids found naturally in the plant Cannabis sativa. Although cannabinoids have been used by humans for thousands of years, it is only in recent years that cannabinoids have been seriously studied for the treatment of a wide array of disorders including insomnia, chronic pain, epilepsy, and post-traumatic stress disorder (Babson et al. (2017) Curr. Psychiatry Rep. 19:23; Romero-Sandoval et al. (2017) Curr. Rheumatol. Rep. 19:67; O'Connell et al. (2017) Epilepsy Behav. 70:341-348; Zir-Aviv et al. (2016) Behav. Pharmacol. 27:561-569). A Cannabis sativa plant may contain over a hundred different cannabinoids which may have different physiological effects. For example, the cannabinoid tetrahydrocannabinol (THC) is responsible for the well-known psychotropic effects of Cannabis extracts, whereas cannabidiol (CBD) lacks these effects but has been demonstrated to reduce inflammation in multiple contexts. Purifying individual cannabinoid species from the Cannabis sativa plant is time-consuming and costly, and results in low yields of many cannabinoid species which may be present as only a small fraction of the total cannabinoid in the plant.

Engineering cells for the production of a specific cannabinoid or cannabinoid precursor would greatly increase the efficiency of obtaining particular cannabinoids. The cannabinoid pathway is complex however, requiring synthesis of both the polyketide olivetolic acid and the isoprenoid geranyldiphosphate (GPP) which are joined by a prenyl transferase to produce the “mother cannabinoid”, cannabigerolic acid (CBGA).

### SUMMARY OF THE DISCLOSURE

Provided herein are cells engineered for the production of cannabinoids, where the engineered cells include one or more genetic modifications that increase cannabinoid production by increasing metabolic flux to cannabinoid precursors and/or reducing carbon losses resulting from the production of byproducts such as pentyl diacetic acid lactone (PDAL). As nonlimiting examples, a genetic modification can be a modification for expressing one or more non-native genes in the engineered host cell or can be a modification for over-expressing or under-expressing one or more endogenous genes in the engineered host cell. Engineered cells as provided herein can include multiple genetic modifications.

Also provided are cell cultures for producing olivetolic acid or a derivative thereof and/or one or more cannabinoids. Derivatives of olivetolic acid include but are not limited to compounds described in International Patent Application publications WO2011127589A1 (e.g. 2,4-dihydroxy-6-heptylbenzoic acid), WO2018209143A1 (e.g. 2-alkyl-4,6-dihydroxy benzoic acid, divarinic acid (i.e., 2-propyl-4,6-dihydroxybenzoic), substituted resorcinols, for example, 5-methylresorcinol, 5-ethylresorcinol, 5-propylresorcinol, 5-hexylresorcinol, 5-heptylresorcinol, 5-octylresorcinol, and 5-nonylresorcinol, and WO2018200888A1 (e.g. olivetolic acid analogs synthesized using CoA compounds). The term “derivative” as used herein includes but is not limited to analogs. The cell cultures include engineered cells as disclosed herein that produce olivetolic acid or a derivative of olivetolic acid and/or one or more cannabinoids in a culture medium that includes a carbon source that can also be an energy source, such as glycerol, a sugar, a sugar alcohol, a polyol, an organic acid, or an amino acid. In various embodiments, the culture medium can include at least one feed molecule such as but not limited to one or more organic acids, amino acids, or alcohols that can be converted into a cannabinoid or olivetolic acid precursor (such as acetyl-CoA, malonyl-CoA, hexanoyl-CoA (or another acyl-CoA molecule), or geranyldiphosphate). Examples of feed molecules include, but are not limited to, bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, decanoate, etc.), a fatty alcohol (e.g., a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc.), prenol, isoprenol and geraniol. Accordingly, “fatty acid” or “carboxylic acid” as used throughout herein includes acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids. Accordingly, “fatty alcohol” as used throughout herein includes a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc. In various embodiments, one, two, three, or more feed molecules can be present in the culture medium during at least a portion of the time the culture is producing olivetolic acid or a derivative thereof or a cannabinoid. Alternatively or in addition, the culture medium can include a supplemental compound that can be a cofactor or a precursor of a cofactor used by an enzyme that functions in a cannabinoid pathway, such as, for example, biotin, thiamine, pantothenate, or 4-phosphopantetheine. A culture medium in some embodiments can include one or more inhibitors of one or more enzymes, such as an enzyme that functions in fatty acid biosynthesis, such as but not limited to cerulenin, thiolactomycin, triclosan, diazaborines such as thienodiazaborine, isoniazid, and analogs thereof.

Further provided are methods for producing cannabinoids that include culturing a cell engineered for the production of olivetolic acid or a derivative thereof or a cannabinoid as provided herein under conditions in which the cell produces olivetolic acid, a derivative thereof, or a cannabinoid. In some examples, the methods include culturing the engineered cells in a culture medium that includes at least one feed molecule or supplement such as but not limited to: bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, decanoate, etc.), a fatty alcohol (includes a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids), prenol, isoprenol, geraniol, biotin, thiamine, pantothenate, and 4-phosphopantetheine in the culture medium during at least a portion of the culture period when the cells are producing olivetolic acid, a derivative thereof, or a cannabinoid. Alternatively or in addition, the methods can optionally include adding one or more fatty acid biosynthesis inhibitors to the culture medium during at least a portion of the culture period when the cells are producing olivetolic acid or a derivative thereof or a cannabinoid. The methods can further include recovering olivetolic acid or a derivative thereof or at least one cannabinoid from the cell, the culture medium, or whole culture. Also provided are cannabinoids produced by the methods provided herein, including derivatives of naturally-occurring cannabinoids, such as, but not limited to, cannabinoid derivatives having different acyl chain lengths than are found in naturally-occurring cannabinoids. The term “derivative” as used herein includes but is not limited to analogs.

In a first aspect, provided herein are cells engineered to produce olivetolic acid or a derivative thereof or a cannabinoid, where the cells include, in addition to nucleic acid sequences encoding polyketide and cyclase activities, one or more genetic modifications for improving production of the olivetolic acid, a derivative of olivetolic acid, or a cannabinoid. As set forth herein, a cell that is engineered to produce olivetolic acid, a derivative thereof or a cannabinoid is engineered to include an exogenous nucleic acid sequence encoding a polyketide synthase that can form 3,5,7-trioxododecanoyl-CoA or 3,5,7-trioxododecanoic acid using malonyl-CoA and hexanoyl-CoA as substrates (e.g., olivetol synthase (OLS), also called 3,5,7-trioxododecanoyl-CoA synthase, EC 2.3.1.206) and to also include an exogenous nucleic acid sequence encoding an olivetolic acid cyclase activity that forms olivetolic acid from 3,5,7-trioxododecanoyl-CoA or 3,5,7-trioxododecanoic acid (e.g., olivetolic acid cyclase, OAC, EC 4.4.1.26) and/or that forms an olivetolic acid derivative via its cyclase activity. In some examples, the polyketide synthase and cyclase activities in the engineered cell may form compounds related to 3,5,7-trioxododecanoyl-CoA or 3,5,7-trioxododecanoic acid and olivetolic acid, i.e., may result in the formation of an olivetolic acid derivative(s) and, optionally a cannabinoid derived therefrom. The cells provided herein that are engineered to produce olivetolic acid or a derivative thereof or a cannabinoid are further engineered to increase the production of the olivetolic acid, olivetolic acid derivative, or cannabinoid product, for example by increasing metabolic flux to a cannabinoid or olivetolic acid pathway, or by decreasing byproduct formation.

In some embodiments, a cell engineered to produce olivetolic acid, a derivative of olivetolic acid, or a cannabinoid is further engineered to increase the supply of coenzyme A (CoA) to increase its availability for producing acetyl-CoA and/or malonyl-CoA as well as hexanoyl-CoA or an alternative acyl-CoA. Strategies for increasing CoA include upregulating endogenous genes that produce CoA from pantothenate: (pantothenate kinase (PanK) (EC 2.7.1.33), phosphopantothenoylcysteine synthetase (PPCS, EC 6.3.2.5), phosphopantothenoylcysteine decarboxylase (PPC-DC, EC 4.1.1.36), phosphopantotheine adenylyl transferase (PPAT), and dephosphocoenzyme A kinase (DPCK)), and/or engineering host cells to include one or more exogenous genes encoding one or more of PanK, PPCS, PPC-DC, PPAT, and DPCK, where the one or more genes are optionally operably linked to heterologous promoters. Alternatively, or in addition, a host cell can be engineered to include a nucleic acid sequence encoding a pantothenate kinase that is not feedback inhibited by coenzyme A, such as a CoaX gene. Cultures of cells engineered for the production of olivetolic acid, a derivative thereof, or a cannabinoid, can in some embodiments include a medium that includes pantothenate, a precursor of CoA, and can optionally also include cysteine, used in CoA biosynthesis. In some embodiments, a culture comprising a host cell engineered for the production of olivetolic acid, a derivative thereof, or a cannabinoid can include 4′-phosphopantotheine in the culture medium and the host cells can be engineered to overexpress genes encoding the enzymes PPAT and DPCK that convert 4′-phosphopantotheine to pantothenate.

Additional strategies for increasing acetyl-CoA, which is converted to the olivetolic acid precursor malonyl-CoA by acetyl-CoA carboxylase (ACC), include expressing an exogenous nucleic acid molecule that encodes an acetyl-CoA synthetase (ACS) that converts acetate and CoA to acetyl-CoA in the host cells. Cultures of engineered host cells that include an exogenous nucleic acid sequence encoding an ACS can optionally include acetate in the culture medium. Examples of CoA synthetases that can be expressed in a host cell engineered to produce a cannabinoid or olivetolic acid or a precursor thereof include, without limitation, the acetyl-CoA synthetase encoded by the acs gene of E. coli (UniprotKB Q8FAY8, SEQ ID NO:11), the ACS of Salmonella typhimurium (UniprotKB Q8Z1R0, SEQ ID NO:12), orthologs of these ACSs in other species, and variants of naturally-occurring ACSs having at least 50% amino acid identity to the naturally-occurring enzymes. Suitable enzymes include acyl-CoA synthetases and CoA ligases. CoA ligases that are not characterized as acetate CoA ligases but have demonstrated activity on acetate include, for example, the Ruegeria pomeroyi 6-carboxy-hexanoate CoA ligase (SEQ ID NO:20) and the 6-carboxy-hexanoate CoA ligase from Burkholderia (SEQ ID NO:21). Candidate CoA ligases for generating acetyl-CoA include the butyrate-CoA ligases of Pseudomonas (CDM46008, SEQ ID NO:93), Streptomyces (Q9X928, SEQ ID NO:96), and bovine (UniProt Q9BEA2, SEQ ID NO::94), Salmonella (SEQ ID NO:28), Candidatus (SEQ ID NO:29), and Homo sapiens (SEQ ID NO:95) and orthologs and variants thereof, the acetoacetate-CoA ligase isolated from rat liver (UniprotKB Q9JMI1, SEQ ID NO:30), and the propanoate-CoA ligases of Ralstonia solanacearum (SEQ ID NO:31) and Salmonella choleraesuis (SEQ ID NO:32). Considered for use in engineered cells that produce a cannabinoid, olivetolic acid, or a derivative of olivetolic acid are the referenced CoA ligases, as well as their orthologs in other species and variants of the referenced CoA ligases and their orthologs having at least 50% identity to the naturally-occurring enzymes.

Also considered, in further embodiments, is an engineered host cell that overexpress a gene encoding a decarboxylating malate dehydrogenase (‘malic enzyme’ EC 1.1.1.38, EC 1.1.1.39, EC 1.1.1.40; maeB in E. coli), or that includes an exogenous gene encoding a decarboxylating malate dehydrogenase, where the malate dehydrogenase converts malate, a TCA cycle intermediate, to pyruvate. Pyruvate can be converted to acetyl-CoA by pyruvate dehydrogenase (PDH), which can optionally also be overexpressed in the host cell. Further, in a prokaryotic host such as E. coli, a variant of the Lpd subunit of PDH can be expressed that includes a mutation (corresponding to A358V in E. coli) that reduces inhibition of PDH by NADH. In some embodiments, a cell culture that includes a host cell that overexpresses pyruvate dehydrogenase can include thiamine in the culture medium.

Alternatively or in addition to strategies for increasing acetyl-CoA, strategies for increasing malonyl-CoA that rely on increased ACC activity can be employed. In some embodiments, the ACC enzyme, which in most eukaryotes, including fungi, is a large single chain polypeptide, and in plant chloroplasts and bacteria such as E. coli is a multi-subunit enzyme, is overexpressed in the host strain, for example, by transformation of the host cell with one or more nucleic acid molecules encoding an ACC or ACC subunit, or by substitution or mutation of promoters regulating the ACC gene(s). In further embodiments, a prokaryotic host cell can be engineered to alter the sequence of one or both of an accA and an accD subunit gene such that the transcripts cannot be bound by the accA and accD polypeptides to avoid inhibition of translation that may otherwise result from such binding. Variants of ACC polypeptides that increase activity are also considered for increasing the malonyl-CoA supply of a host cell as provided herein engineered for the production of OA, a derivative thereof, or a cannabinoid. For example, a eukaryotic ACC can include mutations at amino acid positions corresponding to S659 and/or S1157, of the yeast ACC. In another example, the prokaryotic BCCP subunit polypeptide (encoded by accB in E. coil) can be mutated at position 2, or the nearest acidic residue to the N-terminus to increase ACC activity.

Alternatively or in addition to strategies for increasing acetyl-CoA and strategies for increasing ACC activity, strategies for increasing malonyl-CoA by mechanisms that do not rely on the activity of an ACC can be employed. In some embodiments, a cell that is engineered for the production of olivetolic acid or a derivative thereof or a cannabinoid that is further engineered to increase the cell's supply of malonyl-CoA includes an exogenous nucleic acid sequence that encodes a malonyl-CoA synthetase that generates malonyl-CoA from malonate. Examples of malonyl-CoA synthetases include the malonyl-CoA synthetases of Rhizobium leguminosarum (SEQ ID NO:33), Rhizobium phaseoli (SEQ ID NO:35), or Streptomyces coelicolor (AL163003; SEQ ID NO:36), or a eukaryotic malonyl-CoA synthetase such as the malonyl-CoA synthetase encoded by the AAE13 gene of Arabidopsis (At3g16170; SEQ ID NO:39) or a homolog thereof, or a malonyl-CoA synthetase having at least 50% identity to any of these or other naturally-occurring malonyl-CoA synthetases. Malonate can optionally be added to the culture medium of a culture that includes a cell engineered to express a malonyl-CoA synthetase. An engineered cell that includes an exogenous gene encoding a malonyl-CoA synthetase can also include an exogenous nucleic acid sequence encoding a malonate transporter, such as a malonate transporter encoded by a matC gene, for example of Rhizobium leguminosarum (SEQ ID NO:42), Rhizobium phaseoli (SEQ ID NO:43), or Streptomyces coelicolor (AL163003; SEQ ID NO:44), or a malonate transporter encoded by matL and matM genes that encode subunits of a malonate transporter in species such as Malonomonas rubra, Pseudomonas fluorescens, Burkholderia pseudomallei, and Acinetobacter baumannii.

In additional embodiments, a cell engineered for the production of olivetolic acid or a derivative thereof or a cannabinoid, i.e., a recombinant host cell that includes a polyketide synthase that condenses malonyl-CoA with an acyl-CoA primer and an olivetolic cyclase that forms olivetolic acid or a derivative thereof, is further engineered to include an exogenous nucleic acid sequence that encodes a methylmalonyl-CoA carboxytransferase that can transfer a carboxyl group from oxaloacetate to acetyl-CoA to generate malonyl-CoA. Methylmalonyl-CoA carboxytransferase (MMC) is multimeric enzyme that includes multiple copies of each of a 1.3S subunit, a 5S subunit, and a 12S subunit, each encoded by a separate gene. Exemplary MMC enzymes include, for example, the MMC of Propionibacterium freudenreichii that includes (1.3S subunit, SEQ ID NO:46; 5S subunit, SEQ ID NO:47; and 12S subunit, SEQ ID NO:48); the MMC of Clostridium cellulolyticum (1.3S subunit, SEQ ID NO:49; 5S subunit, SEQ ID NO:50; and 12S subunit, SEQ ID NO:51); and the MMC of Geobacter bemidjiensis (1.3S subunit, SEQ ID NO:61; 5S subunit, SEQ ID NO:62; and 12S subunit, SEQ ID NO:63), as well as orthologs of these enzymes in other species, and variants of naturally-occurring MMC subunits having at least 50% amino acid sequence identity thereto. An engineered cell that expresses an exogenous nucleic acid sequence encoding a methylmalonyl-CoA carboxytransferase can also optionally be engineered to express or overexpress a nucleic acid sequence that encodes a polypeptide whose activity increases production of oxaloacetate, such as for example an aspartate transaminase. A cell culture that includes a cell engineered for the production of olivetolic acid or a cannabinoid or a derivative of either and that expresses a nucleic acid sequence encoding a methylmalonyl-CoA carboxytransferase can in some embodiments be cultured in a medium that includes one or both of oxaloacetate and aspartate.

In further embodiments, a cell engineered for the production of olivetolic acid or a derivative thereof or a cannabinoid is further engineered to include an exogenous nucleic acid sequence that encodes a malonyl-CoA reductase (MCR, EC 1.2.1.75) that is able to convert malonyl semialdehyde to malonyl-CoA. A cell engineered to express a malonyl-CoA reductase can further include one or more nucleic acid sequences encoding one or more enzymes for generating malonyl semialdehyde from beta-alanine. For example, a cell engineered to express an exogenous malonyl-CoA reductase can also express an exogenous nucleic acid sequence encoding a beta-alanine pyruvate transaminase (EC 2.6.1.18) that generates malonyl semialdehyde from beta-alanine. Examples of BAPATs that can be expressed in an engineered cell as provided herein include, without limitation, various BAPATs of Pseudomonas species, including for example, the BAPAT of Pseudomonas putida (SEQ ID NO:64), the BAPAT of Pseudomonas chlororaphis subsp. aureofaciens (SEQ ID NO:69), and the BAPAT of Pseudomonas asplenii (SEQ ID NO:73) orthologs thereof in other species, and variants of these and other naturally-occurring BAPATs having at least 50% identity thereto. A host cell engineered to include an exogenous gene encoding a BAPAT can optionally be further engineered to express an exogenous nucleic acid sequence encoding and aspartate 1-decarboxylase (EC 4.1.1.11) or to overexpress an endogenous aspartate 1-decarboxylase that converts aspartate to beta-alanine. Cultures for producing olivetolic acid or a derivative thereof or a cannabinoid that include a cell engineered to convert beta-alanine to malonyl-CoA, i.e., that is engineered to express an exogenous BAPAT and MCR, and optionally to express or overexpress an aspartate 1-decarboxylase, can include one or a combination of beta-alanine, aspartate, or oxaloacetate in the culture medium.

A cell engineered to include a pathway from beta-alanine to malonyl-CoA can further include one or more enzymes for providing NADP+, used in the malonyl-CoA reductase reaction, such as for example, a water-producing NADPH oxidase. One example of a NADPH oxidase that can be expressed in an engineered cell as provided herein is the variant NADH oxidase from Lactobacillus brevis (SEQ ID NO:74) that has an altered substrate specificity with regard to the native enzyme. NADH oxidases can be mutated, for example, at amino acid positions corresponding to G159A, D177A, A178R, M179S, and P184R of SEQ ID NO:74, to convert their substrate specificity from NADH to NADPH. Additional enzymes that can be modified to include the same mutations include, for example, the NADH oxidase of Borrelia burgdorferi, and the NADH oxidases of Lactobacillus reuteri (WP_003669593; SEQ ID NO:78), Lactobacillus antri (EEW53553; SEQ ID NO:85), and Lactobacillus vaccinostercus (WP_057777538), as well as variants of these enzymes and orthologs in other species having at least 50% identity thereto.

In yet further embodiments, a cell that is engineered to produce a cannabinoid or olivetolic acid or a derivative thereof, can also be engineered to express or overexpress a polypeptide that functions in a pathway that produces hexanoyl-CoA or another acyl-CoA that can be used as a precursor for making an olivetolic acid derivative. The recombinant cell can be further modified to express or overexpress an acyl-CoA ligase, an acyl-activating enzyme (AAE), or a fatty acyl-CoA synthetase (FAA) that generates acyl-CoA from a fatty acid, propionate, butyrate, valerate or isovalerate. In some examples, the acyl-CoA ligase, AAE, or FAA converts hexanoate to hexanoyl-CoA. In additional examples, where the engineered cell produces a derivative of olivetolic acid or a cannabinoid synthesized therefrom, the acyl-CoA ligase, AAE, or FAkcan convert acetate, propionate, butyrate, valerate, or isovalerate or a fatty acid including acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids, to the corresponding acyl-CoA. In various embodiments acyl-CoA ligases expressed in the engineered cells provided herein can use fatty acids of varying chain lengths as well acetate and/or malonate as substrates. In some examples, the engineered host cell expresses the Ruegeria pomeroyi CoA ligase (SEQ ID NO:20) or the Burkholderia CoA ligase (SEQ ID NO:21), or an acyl-CoA ligase having at least 50% amino acid identity thereto, where the CoA ligase produces acetyl-CoA as well as hexanoyl-CoA, and/or other acyl-CoAs as described above and herein include CoA derivatives of acetate, propionate, butyrate, valerate, or isovalerate or a fatty acid of a chain length other than C6, propionate, butyrate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids, etc.

In various embodiments of a culture that produces olivetolic acid or a derivative thereof or a cannabinoid, including but not limited to embodiments where the host cells are engineered to express or overexpress an acyl-CoA ligase, AEE, or FAA, hexanoic acid or another fatty acid can be provided in the culture medium. As noted herein, fatty acids can include acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids. In various embodiments of a culture that produces olivetolic acid or a derivative thereof or a cannabinoid, including but not limited to embodiments where the host cells are engineered to express or overexpress an acyl-CoA ligase, AEE, or FAA, hexanoic acid can be produced by the host cells, for example, by a fatty acid biosynthesis pathway, or by an engineered reverse beta-oxidation pathway. In some embodiments where hexanoyl-CoA (or another acyl-CoA) is produced by the host cell, the host cell can optionally be engineered for expression or overexpression of a thioesterase that acts on a C6 acyl-thioester, or an acyl-thioester having a different carbon chain length.

In some embodiments, a host cell engineered for the production of a cannabinoid is also engineered to overexpress a native alcohol dehydrogenase or to include an exogenous nucleic acid molecule encoding an alcohol dehydrogenase, and/or engineered to overexpress a native aldehyde dehydrogenase or to include an exogenous nucleic acid molecule encoding an aldehyde dehydrogenase, where the overexpressed or introduced alcohol dehydrogenase is able to convert hexanol to hexanal, and the hexanal is converted to the corresponding hexanoyl-CoA by the aldehyde dehydrogenase. Or where variants or olivetolic acid or a cannabinoid having different hydrocarbon chains are produced, an alcohol dehydrogenase that is expressed or overexpressed in the cell can convert, for example, any fatty alcohol to the its corresponding fatty aldehyde, and the fatty aldehyde is converted to the corresponding fatty acyl-CoA by the aldehyde dehydrogenase. In some embodiments the alcohol dehydrogenase and the aldehyde dehydrogenase activity reside in the same dual-functional enzyme, encoded by the same gene. In some embodiments the alcohol dehydrogenase is a native alcohol dehydrogenase, such as the alcohol dehydrogenase encoded by adhE of E. coli, Clostridium acetobutylicum or by ADH1, ADH2, or ADH6 of Saccharomyces, or AdhE2 and its homologs, e.g. from E. coli and from Clostridium acetobutylicum (UniProtKB-Q9ANR5), to catalyze alcohol to aldehyde and subsequent acyl-CoA formation. In some embodiments, one or more adhE genes is introduced into an E. coli host cell under the control of a constitutive or inducible promoter.

The host cell engineered to express or overexpress an alcohol dehydrogenase and aldehyde dehydrogenase activities for converting a fatty alcohol to a fatty acyl-CoA can further be engineered to express or overexpress acyl-CoA ligase, AAE, or FAA for converting any fatty acid to an acyl-CoA molecule. The fatty acid may arise from endogenous enzymes acting on the fatty aldehyde converting it to a fatty acid. In some embodiments the cells are engineered to delete or reduce expression enzymes that act upon and convert the aldehyde intermediate to its acid. Hexanol or the fatty alcohol of the desired chain length may be provided to the cells in the cell culture medium.

In another aspect, cells engineered to produce a cannabinoid, olivetolic acid, or a derivative of olivetolic acid are further engineered to reduce the accumulation of the byproduct pentyl diacetic acid lactone (PDAL; 3,5-dioxodecanoic acid), hexanoyl triacetic acid lactone (HTAL), or analogs thereof depending on which olivetolic acid derivative is produced. In some embodiments, the host cells are engineered to disrupt or delete one or more endogenous genes encoding lactone transporters that may transport PDAL, HTAL or other OA derivative byproduct molecules out of the cell. Candidates for genes encoding transporters that can export a lactone such as PDAL out of the cell include FadL and mexA-mexB-oprM genes of prokaryotic hosts. In some embodiments, the host cells are engineered to produce a lactonase that hydrolyzes the lactone ring of PDAL, HTAL or other OA derivative byproduct to generate the PDAL diketide, HTAL triketide hydrolysis products or other corresponding polyketide hydrolysis product, such as, for example, a lactonase derived from a plant, animal, or bacterium. As nonlimiting examples, a lactonase expressed by an engineered cell as provided herein can be a acyl-homoserine lactonase, 1,4-lactonase, 2-pyrone-4,6-dicarboxylate lactonase, 3-oxoadipate enol-lactonase, actinomycin lactonase, deoxylimonate A-ring lactonase, gluconolactonase, L-rhamnono-1,4-lactonase, limonin-D-ring-lactonase, steroid lactonase, triacetate-lactonase, or a xylono-1,4-lactonase. For example, the engineered host cell in some embodiments can include an exogenous gene encoding an acyl-homoserine lactonase, such as but not limited to the AiiA_{240B1} (AHL inactivating) lactonase of a Bacillus species (e.g., Q9L8R8, SEQ ID NO:97), the AttM (Q8VPD5, SEQ ID NO: 98) and AiiB (CUX06432, SEQ ID NO:99) lactonases of Agrobacterium, and the AiiD gene of a Ralstonia species (e.g., WP_064051080, SEQ ID NO:100), as well as orthologs of any of these lactonases and variants having at least 50% identity to these lactonases and their naturally-occurring orthologs.

In various examples of these embodiments the host cell also expresses a CoA ligase that converts the hydrolyzed PDAL molecule, referred to herein as a diketide, to a diketide-CoA the hydrolyzed HTAL molecule, referred to herein as a triketide, to a triketide-CoA, or the other hydrolyzed OA derivative byproduct to its polyketide-CoA. Examples of CoA ligases include those referenced above and throughout this disclosure, including without limitation the Ruegeria pomeroyi CoA ligase (SEQ ID NO:20) or the Burkholderia CoA ligase (SEQ ID NO:21) that have activity on acetyl-CoA as well as octanoyl-CoA, orthologs thereof, or an acyl-CoA ligase having at least 50% amino acid identity to either. A malonyl-CoA synthetase is another class of suitable enzymes. Examples of a malonyl-CoA ligase include those referenced throughout this disclosure, including without limitation that of R. trifolii (JACS, 123:5822-5823 (2001)) and the orthologs thereof having at least 50% amino acid identity. Without limiting the invention to any particular mechanism, in some embodiments the recovered CoA-thioesterified di- and/or triketide or polyketide resulting from PDAL, HDAL or other OA derivative byproduct hydrolysis, e.g. 3,5-dioxodecanoyl-CoA intermediate, can be acted on by biosynthetic enzymes such as a polyketide synthase and optionally an olivetolic acid cyclase to generate olivetolic acid, thus recycling carbons that would otherwise be lost in an exported byproduct. For example, in the case of PDAL, the CoA-thiesterified diketide resulting from PDAL hydrolysis can be acted on by OLS to condense a third molecule of malonyl-CoA to generate triketide CoA, which would be acted upon by biosynthetic enzymes such as a polyketide synthase and optionally an olivetolic acid cyclase to generate olivetolic acid, thus recycling carbons that would otherwise be lost in an exported PDAL byproduct.

In further aspects a cell engineered to produce a cannabinoid can be engineered to express or overexpress one or more nucleic acid sequences encoding an enzyme or enzymes that are involved in converting prenol and/or isoprenol to geranyl diphosphate (GPP) as is known to occur through a set of reactions. As described in detail below, prenol is phosphorylated to DMAP and isoprenol to IP. DMAP is phosphorylated to DMAPP and IP to IPP. DMAPP and IPP can be interconverted via an isomerase. DMAPP and IPP are reacted together to produce GPP. For example, a cell engineered to produce a cannabinoid can include a nucleic acid sequence encoding an alcohol kinase, such as, for example, the E. coli alcohol kinase YchB (NP_415726, SEQ ID NO:101) or the Thermoplasma acidophilum phosphate kinase ThaIPK having the mutations V73I, Y141V, and K204G that increase activity on prenol (WP_010900530, SEQ ID NO:102) or orthologs thereof, or variants of these kinases and their orthologs having at least 50% identity thereto that convert prenol or isoprenol to dimethylallyl phosphate (DMAP) or isopentenyl phosphate (IP). respectively. The engineered cells can further include an exogenous nucleic acid sequence encoding a prenyl phosphate kinase (or isopentenyl phosphate kinase, IPK) that can convert DMAP or IP to dimethylallyl pyrophosphate (DMAPP) or IP to isopentenyl pyrophosphate (IPP), respectively, such as, for example, the Methanothermobacter thermautotrophicus prenyl phosphate kinase MtIPF (AAB84554), orthologs thereof, or variants having at least 50% identity to naturally-occurring phosphate kinases. Additionally, an engineered cell can include an exogenous nucleic acid sequence encoding an alcohol diphosphoisomeraase such as the E. coli isopentenyl pyrophosphate isomerase (NP_4173365) that converts DMAPP to IPP. The cells engineered for cannabinoid production can further include a GPP synthase, such as E. coli IspA (NP_414955, SEQ ID NO:103), C. glutamicum IdsA (WP_011014931.1, SEQ ID NO:104) or a GPP synthase from Abies Grandis (GPPS2; AAN01134, with 84 amino acid N-terminal truncation, SEQ ID NO:105). GPP synthases that are orthologs of these examples of GPP synthases or are variants of these GPP synthases or their orthologs having at least 50% amino acid sequence identity thereto are also considered for use in a host cell as provided herein.

In other examples, a cell engineered to produce a cannabinoid can include a nucleic acid sequence encoding an alcohol kinase that converts geraniol to geranyl monophosphate and can further include a nucleic acid sequence encoding a geranyl phosphate kinase (GPK) that converts geranyl monophosphate to GPP. The farnesol kinase (FOLK) of Arabidopsis thaliana (UniprotKB Q67ZM7, SEQ ID NO:106) is an example of a geraniol kinase that can be expressed in an engineered host cell for conversion of geraniol to geranyl monophosphate. Also considered are farnesol kinases that are orthologs of SEQ ID NO:106 and have geraniol kinase activity, as well as variants having at least 50% identity to SEQ ID NO:106 and its orthologs. Examples of geranyl phosphate kinases that can be expressed in a cell engineered for the production of cannabinoids include the isopentenyl phosphate kinase of Thermoplasma acidophilum with mutated amino acids at positions 70 and 140, optionally in combination with mutations at amino acid positions 73 and/or 130, and the isopentenyl phosphate kinase of Methanothermobacter thermoautotrophicus with mutated amino acids at positions 77, 142, and 154. Also considered are orthologs of the Thermoplasma and Methanothermobacter isopentenyl phosphate kinases, including variants of orthologs with mutations at the corresponding positions, as well as isopentenyl phosphate kinases having at least 50% identity to the Thermoplasma and Methanothermobacter isopentenyl phosphate kinases.

A cell engineered to produce a cannabinoid or a cannabinoid derivative can also be modified to disrupt or delete endogenous genes that encode hydrolases that can dephosphorylate geranyldiphosphate or geranyldiphosphate precursors such as IPP and DMPP. For example, in E. coli the nudix hydrolase genes NudB, NudF, Nudl, and NudJ (EC 3.6.1) can be downregulated or disrupted, and in Saccharomyces cerevisiae, the Ysa1p gene can be downregulated or disrupted. Orthologous genes in other host species can be similarly downregulated or disrupted. Alkaline phosphatase genes of E. coli, such as, for example, PhoA, AphA, BacA, PgpA, and/or PgpB can also be deleted, disrupted, or downregulated. Where the engineered host cell is a eukaryotic cell such as a fungal cell, a diacylglycerol diphosphate phosphatase gene or a lipid phosphate phosphatase gene such as DPP1 and LPP1, respectively in Saccharomyces cerevisiae or orthologous genes in other eukaryotes can be downregulated or disrupted to prevent hydrolysis of GPP or its precursors.

A host cell engineered for the production of a cannabinoid can include a prenyltransferase that uses GPP to prenylate olivetolic acid resulting in the production of cannabigerolic acid (CBGA). Examples of prenyltransferases that can be expressed in a host cell include, without limitation, the C. sativa geranyl-pyrophosphate-olivetolic acid geranyltransferase (GOT, EC 2.5.1.102) and orthologs thereof, as well as variants of the GOT of C. sativa and orthologs thereof having at least 50% identity to a naturally-occurring geranyl-pyrophosphate-olivetolic acid geranyltransferase. Also considered for expression in host cells engineered for producing cannabinoids are aromatic prenyltransferases of bacterial species such as the Streptomyces NphB gene product and variants thereof having at least 50% identity to a naturally-occurring aromatic prenyltransferase.

A host cell engineered for the production of a cannabinoid can optionally include an exogenous gene encoding a cannabinoid synthase that can be used to produce particular cannabinoids. Nonlimiting examples of cannabinoid synthases include a C. sativa tetrahydrocannabinolic acid (THCA) synthase polypeptide and a C. sativa cannabidiolic acid (CBDA) synthase polypeptide as well as derivatives thereof having at least 50% identity to naturally-occurring cannabinoid synthases.

Additional genetic modifications that may be present in a host cell engineered to produce olivetolic acid or a derivative thereof or a cannabinoid include downregulation, disruption, or deletion of genes encoding alcohol dehydrogenase, lactate dehydrogenase, acetyl phosphate transferase and acetate kinase enzymes that divert acetate and acetyl-CoA to ethanol, lactate, and acetyl phosphate. In an E. coli host cell, genes that are downregulated, disrupted, or deleted can include adhE, ldhA, and ack-pta. Further, a cell engineered for the production of olivetolic acid or a cannabinoid can have one or more genes encoding thioesterases downregulated, disrupted, or deleted to prevent hydrolysis of olivetolic acid precursors hexanoyl-CoA and malonyl-CoA (as well as acetyl-CoA). For example, in an E. coli host one or more of the thioesterases genes FadM (P_414977.1), TesA (P_415027.1), TesB (P_414986.1), YciA (P_415769.1), Ydil (P_416201.1) and YbgC (P_415264.1) can be downregulated, disrupted, or deleted. In various embodiments a culture of a host cell engineered to have reduced or eliminated activity of one or more endogenous thioesterases can include hexanol or another fatty alcohol (including a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids) or hexanoate or other fatty acid (including acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids), or alkanoate provided in the culture medium during at least a portion of the time the cells are producing olivetolic acid, a derivative thereof, or a cannabinoid.

Reducing fatty acid degradation may also be desirable for reducing degradation of the hexanoyl-CoA (or other acyl-CoA) precursor. In E. coli for example, the FadE gene (encoding acyl-CoA dehydrogenase) can be downregulated, disrupted, or deleted. Other modifications to endogenous genes that may improve olivetolic acid or cannabinoid synthesis include mutation or downregulation of one or more genes that function in fatty acid biosynthesis. Without limiting the embodiments to any particular mechanism, limiting fatty acid biosynthesis can increase the malonyl-CoA supply available for cannabinoid biosynthesis. In some embodiments, a temperature-sensitive variant of the enoyl-CoA reductase (E. coli FabI) can be engineered into a host cell to result in diminished fatty acid biosynthesis at the non-permissive temperature. Another example of a fatty acid biosynthesis gene of a host cell that may be mutated or downregulated is a gene encoding a beta-ketoacyl-ACP synthase I enzyme (E. coli FabB). Other fatty acid biosynthesis endogenous genes of the engineered host cell that can be mutated to result in an enzyme of diminished function and thereby increase the malonyl-CoA supply include the gene beta-ketoacyl-ACP synthase III (E. coli FabH) and the gene encoding malonyl-CoA-ACP transacylase (E. coli FabD). In some examples, a gene encoding a beta-ketoacyl-ACP synthase II enzyme (E. coli FabF) can be overexpressed to reduce fatty acid biosynthesis.

Alternatively or in addition, genes encoding enzymes of the tricarboxylic acid cycle, such as succinate dehydrogenase, or the gene encoding citrate synthase that converts acetyl-CoA to citrate, which feeds into the TCA, can be disrupted or downregulated.

Engineered cells that produce a cannabinoid or olivetolic acid or a derivative thereof, can be engineered to include multiple pathways to enhance olivetolic acid or cannabinoid production. Those skilled in the art will recognize that the embodiments described herein can be combined in multiple ways. Examples of engineered cells having multiple genetic modifications .are exemplary only and do not limit the scope of the invention.

In another aspect, provided herein are cell cultures that include engineered cells as provided herein in a culture medium, where the culture medium includes a carbon source that is also an energy source for the cells, where the carbon source can be, for example, glycerol, a sugar, sugar alcohol, polyol, organic acid, or amino acid, as nonlimiting examples. The culture medium can further include a feed molecule that is used to produce the olivetolic acid or cannabinoid product. A feed molecule can be, for example, acetate, malonate, oxaloacetate, aspartate, beta-alanine, alpha-alanine, hexanoate or an alternative fatty acid (including acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids), alkanoate, prenol, isoprenol, or geraniol. In some embodiments, a feed molecule that can be converted into the olivetolic acid or cannabinoid product is also a carbon and energy source for growth of the culture. In some embodiments, the culture comprises a culture medium that includes a carbon source and at least one supplement compound that is a cofactor of an enzyme or is a precursor of an enzyme cofactor. In some embodiments, a culture includes an inhibitor of fatty acid biosynthesis, such as, but not limited to, cerulenin, thiolactomycin, triclosan, diazaborines such as thienodiazaborine, isoniazid, and analogs thereof.

In yet another aspect, methods for producing olivetolic acid, a derivative of olivetolic acid, or a cannabinoid that include incubating a culture of an engineered host cell as provided herein to produce olivetolic acid, a derivative of olivetolic acid, or a cannabinoid. The methods can further include recovering the olivetolic acid, or cannabinoid from the cells, the culture medium, or whole culture.

Further provided are cannabinoids produced using the engineered cells, cultures, and methods provided herein. A cannabinoid produced using the cells and methods provided herein can be any disclosed herein, or a prodrug thereof, including any disclosed in WO2017181118, incorporated herein by reference in its entirety.

### BRIEF DESCRIPTION OF THE DRAWINGS

FIG. 1 provides structures of olivetolic acid and geraniol.

FIGS. 2A and B provide structures of cannabinoids.

### DETAILED DESCRIPTION OF THE DISCLOSURE

The present application provides engineered cells for producing olivetolic acid, an olivetolic acid derivative, or at least one cannabinoid, as well as cultures that include the engineered cells and methods of producing olivetolic acid, an olivetolic acid derivative, or at least one cannabinoid. Olivetolic acid is the polyketide precursor of cannabinoids that is prenylated to produce cannabigerolic acid (CBGA), a cannabinoid that can be converted into a number of different cannabinoids, for example, by the action of a cannabinoid synthase.

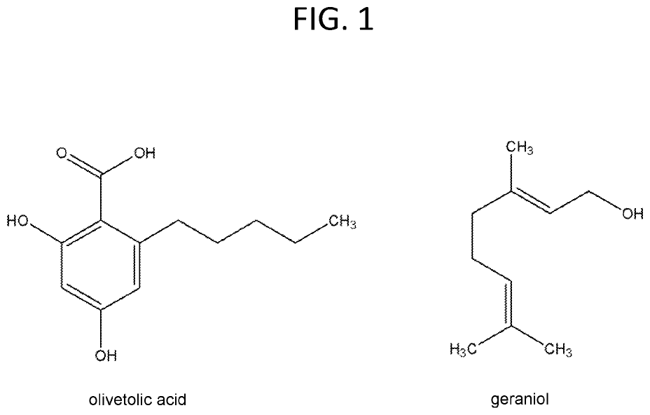

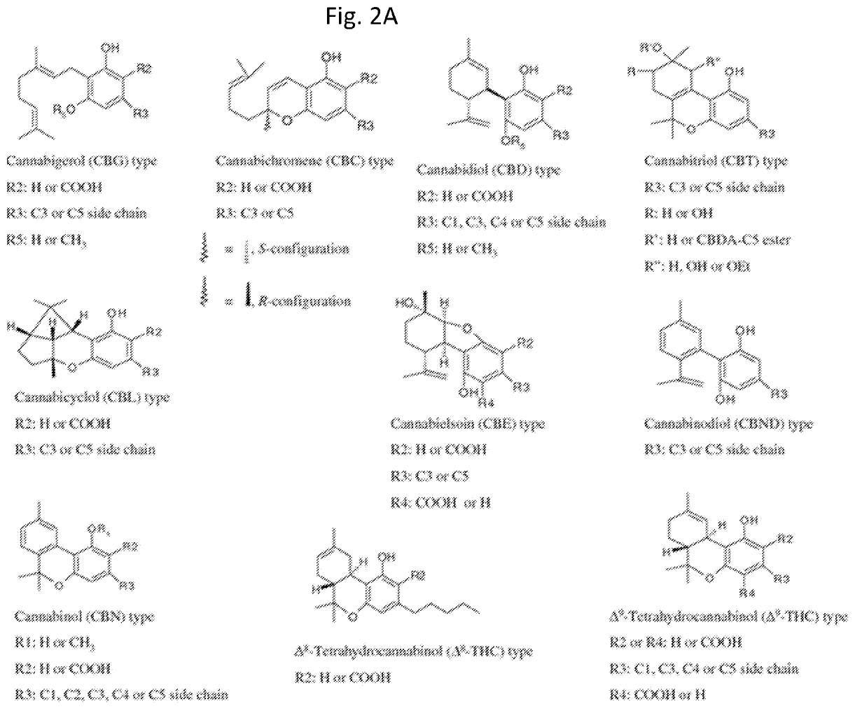

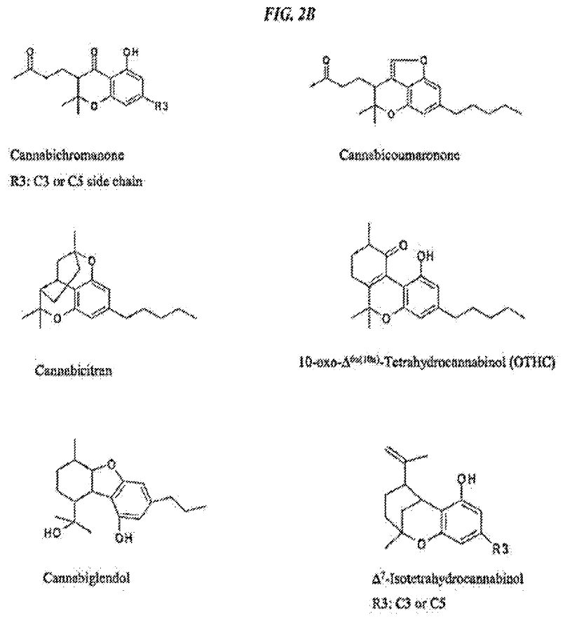

The terms “cannabinoid”, “cannabinoid product”, and “cannabinoid compound” or “cannabinoid molecule” are used herein to refer to a member of a class of meroterpenoids that are naturally-occurring in Cannabis sativa. As used herein, the terms “cannabinoid”, “cannabinoid product”, and “cannabinoid compound” or “cannabinoid molecule” are used interchangeably to refer a molecule containing a polyketide moiety, e.g., olivetolic acid or another 2-alkyl-4,6-dihydroxybenzoic acid, and a terpene-derived moiety e.g., a geranyl group (FIG. 1). Geranyl groups are derived from the diphosphate of geraniol, known as geranyl-diphosphate or geranyl-pyrophosphate that forms the acidic cannabinoid cannabigerolic acid (CBGA). CBGA can be converted to further bioactive cannabinoids both enzymatically (e.g., by decarboxylation via enzyme treatment in vivo or in vitro to form the neutral cannabinoid cannabigerol) and chemically (e.g., by heating); see also, FIGS. 2A and 2B.

The term cannabinoid includes acid cannabinoids and neutral cannabinoids. The term cannabinoids also includes derivatives of naturally-occurring cannabinoids, such as, but not limited to, cannabinoids having different alkyl chain lengths of side groups than are found in naturally-occurring cannabinoids. The term “acidic cannabinoid” generally refers to a cannabinoid having a carboxylic acid moiety. The carboxylic acid moiety may be present in protonated form (i.e., as —COOH) or in deprotonated form (i.e., as carboxylate Examples of acidic cannabinoids include, but are not limited to, cannabigerolic acid, cannabidiolic acid, and Δ⁹-tetrahydrocannabinolic acid. The term “neutral cannabinoid” refers to a cannabinoid that does not contain a carboxylic acid moiety (i.e., does contain a moiety —COOH or —COO⁻). Examples of neutral cannabinoids include, but are not limited to, cannabigerol, cannabidiol, and Δ⁹-tetrahydrocannabinol.

Throughout the applications, abbreviated terms may be used to designate cannabinoids and related molecules. For example, the term “CBGA” refers to cannabigerolic acid, “OA” refers to olivetolic acid; “CBG” refers to cannabigerol; “CBDA” refers to cannabidiolic acid; “CBD” refers to cannabidiol; “THC” refers to Δ⁹-tetrahydrocannabinol (Δ⁹-THC); “Δ⁸-THC” refers to Δ⁸-tetrahydrocannabinol; “THCA” refers to Δ⁹-tetrahydrocannabinolic acid (Δ⁹-THCA); “Δ⁸-THCA” refers to Δ⁸-tetrahydrocannabinolic acid; “CBCA” refers to cannabichromenic acid; “CBC” refers to cannabichromene; “CBN” refers to cannabinol; “CBDN” refers to cannabinodiol; “CBNA” refers to cannabinolic acid; “CBV” refers to cannabivarin; “CBVA” refers to cannabivarinic acid; “THCV” refers to Δ⁹-tetrahydrocannabivarin (Δ⁹-THCV); “Δ⁸-THCV” refers to Δ⁸-tetrahydrocannabivarin; “THCVA” refers to Δ⁹-tetrahydrocannabivarinic acid (Δ⁹-THCV); “Δ⁸-THCVA” refers to Δ⁸-tetrahydrocannabivarinic acid; “CBGV” refers to cannabigerovarin; “CBGVA” refers to cannabigerovarinic acid; “CBCV” refers to cannabichronievarin; “CBCVA” refers to cannabichromevarinic acid; “CBDV” refers to cannabidivarin; “CBDVA” refers to cannabidivarinic acid; “MPF” refers to multiple precursor feeding; “PKS” refers to a polyketide synthase; “GOT” refers to geranyl pyrophosphate olivetolate geranyl transferase; and “HPLC” refers to high performance liquid chromatography.

Cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), A⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

Cannabinoid compounds of interest include, without limitation, CBG, CBDA, CBD, THC, Δ⁸-THC, THCA, Δ⁸-THCA, CBCA, CBA, CBN, CBDN, CBNA, CBV, CBVA, THCV, THCVA, Δ⁸-THCA, CBGV, CBGVA, CBCV, CBCVA, CBDV and CBDVA and derivatives thereof. The term “derivatives” as used herein includes but is not limited to analogs. Given the high levels of products obtained using the novel manufacturing systems created by the present invention, also of interest are some less well-studied cannabinoids that may have more potent and selective activities in various human medical conditions. They include, without limitation, the cannabichromanones, cannabicoumaronone, cannabicitran, 10-oxo-Δ^{6a(10a)}-tetrahydrohydrocannabinol (OTHC), cannabiglendol, and Δ⁷-isotetrahydrocannabinol, whose structures are shown in FIG. 2B.

“Cannabinoid precursor” as used herein may refer to any intermediate present in the cannabinoid biosynthetic pathway before the production of the “mother cannabinoid,” cannabigerolic acid (CBGA). Cannabinoid precursors may include, but are not limited to, pyruvate, acetyl-CoA, malonyl-CoA, hexanoyl-CoA, acetoacetyl-CoA, 3,5,7-trioxododecanoyl-CoA, olivetolic acid, prenol, isoprenol, DMAP, IF, DMAPP, IPP, GPP, butyryl-CoA, HMG-CoA, mevalonate, mevalonate-5-phosphate, and mevalonate diphosphate.

Cells engineered for the production of cannabinoids (or olivetolic acid or another) can have one or multiple modifications, including, without limitation, the downregulation, disruption, or deletion of endogenous genes, the upregulation of an endogenous gene, and the introduction of exogenous genes.

The term “non-naturally occurring”, when used in reference to an organism (e.g., microbial) or host cell is intended to mean that the organism or host cell has at least one genetic alteration not normally found in a naturally occurring organism of the referenced species that is the result of human intervention. Naturally-occurring organisms can be referred to as “wild-type” such as wild type strains of the referenced species. Likewise, a “non-natural” polypeptide or nucleic acid can include at least one genetic alteration not normally found in a naturally-occurring polypeptide or nucleic acid. Naturally-occurring organisms, nucleic acids, and polypeptides can be referred to as “wild-type” or “original” such as wild type strains of the referenced species. Likewise, amino acids found in polypeptides of the wild type organism can be referred to as “original” with regards to any amino acid position. A “non-naturally-occurring” host cell, organism, or microorganism that includes at least one genetic medication generated by human intervention can also be referred to as “engineered”, “genetically engineered”, or “recombinant”.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene, a nucleic acid, nucleic acid sequence, or nucleic acid molecule, or to overexpress an enzyme or polypeptide has been genetically engineered through recombinant DNA technology to include a gene or nucleic acid sequence that it does not naturally include that encodes the enzyme or polypeptide or to express an endogenous gene at a level that exceeds its level of expression in a nonaltered cell. As nonlimiting examples, a host cell, organism, or microorganism engineered to express or overexpress a gene, a nucleic acid, nucleic acid sequence, or nucleic acid molecule, or to overexpress an enzyme or polypeptide can have any modifications that affect a coding sequence of a gene, the position of a gene on a chromosome or episome, or regulatory elements associated with a gene. Overexpression of a gene can also be by increasing the copy number of a gene in the cell or organism. Similarly, a host cell, organism, or microorganism engineered to under-express (or to have reduced expression of) a gene, nucleic acid, nucleic acid sequence, or nucleic acid molecule, or to under-express an enzyme or polypeptide can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express a gene also include modifications to regulatory regions of the gene that can reduce its expression.

The term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material that may be introduced on a vehicle such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is naturally present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host species.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid/nucleic acid sequence. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Genes or nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organdies, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The percent identity (% identity) between two sequences is determined when sequences are aligned for maximum homology, and not including gaps or truncations as set forth in the BLAST parameters. Exemplary parameters for determining relatedness of two or more amino acid sequences using the BLAST algorithm, for example, can be as provided in BLASTP using the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, for determining the relatedness of two or more sequences. Additional sequences added to a polypeptide sequence, such as but not limited to immunodetection tags, purification tags, localization sequences (presence or absence), etc., do not affect the % identity.

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%).

For example, alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (http://balign.sourceforge.net/). Default parameters are used for the alignment and BLOSUM62 was used as the scoring matrix. In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

“Conservative amino acid substitution” or, simply, “conservative variations” of a particular sequence refers to the replacement of one amino acid, or series of amino acids, with essentially identical amino acid sequences. One of skill will recognize that individual substitutions, deletions or additions that alter, add or delete a single amino acid or a percentage of amino acids in an encoded sequence result in “conservative variations” where the alterations result in the deletion of an amino acid, addition of an amino acid, or substitution of an amino acid with a functionally similar amino acid. One of skill in the art will also recognize that an individual substitution, deletion or addition disclosed herein that refers to a specific variant, e.g. substitution of a Glycine a residue 34 with a Histidine (G34H), can include a conservative substitution made at the same residue as disclosed herein, e.g. instead of a Histidine being substituted at residue 34, an Arginine (R) or a Lysine (K) can be substituted.

Conservative substitution tables providing functionally similar amino acids are well known in the art. Conservative amino acid substitutions includes replacement of one amino acid with another having the same type of functional group or side chain polarity, size, shape or charge (e.g., aliphatic, aromatic, positively charged, negatively charged, polar, non-polar, positive polar, negative polar, uncharged polar, non-polar hydrophobic, ionizable acidic, ionizable basic, or sulfur containing residues).

As a non-limiting example, the following six groups each contain amino acids that can be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K); Histidine (H);

5) Isoleucine (I), Leucine (L), Methionine (M), Valine (V); and

6) Phenylalanine (F), Tyrosine (Y), Tryptophan (W).

Thus, conservative amino acid substitutions of a polypeptide sequence disclosed herein can include substitutions of a percentage, such as less than 1%, 5% or 10%, of the amino acids of the polypeptide sequence, with a conservatively selected amino acid of the same conservative substitution group. Additionally, a conservatively substituted variation of a polypeptide sequence disclosed herein can contain no more than 1, 2, 3, 4, 5, 10, 20, 30, 50, or 100 substitutions with a conservatively substituted variation of the same conservative substitution group.

It is also understood that the addition or substitution of nucleic acid sequences which do not alter the encoded polypeptide activity of a nucleic acid molecule, such as the addition of a non-functional or non-coding sequence, is a conservative variation of the nucleic acid sequence. One of skill in the art will appreciate that many conservative variations of the nucleic acid molecules disclosed yield a functionally identical polypeptide. For example, owing to the degeneracy of the genetic code, silent substitutions (i.e., substitutions in a nucleic acid sequence which do not result in an alteration in an encoded polypeptide) are an implied feature of every nucleic acid sequence which encodes an amino acid. Similarly, conservative amino acid substitutions, in one or more amino acids in an amino acid sequence can be substituted with different amino acids with highly similar properties, are also readily identified as being highly similar to a disclosed construct. Such conservative variations of each disclosed sequence are a feature of the polypeptides disclosed herein.

Non-conservative modifications of a particular polypeptide are those which substitute any amino acid not characterized as a; conservative substitution. For example, any substitution which crosses the bounds of the six groups set forth above. These include substitutions of basic or acidic amino acids for neutral amino acids (e.g., Aspartic acid (D), Glutamic acid (E), Asparagine (N), or Glutamine (Q) for Valine (V), Isoleucine (I), Leucine (L) or Methionine (M)), aromatic amino acid for basic or acidic amino acids (e.g., Phenylalanine (F), Tyrosine (Y) or Tryptophan (W) for Aspartic acid (D), Asparagine (N), Glutamic acid (E) or Glutamine (Q)), or any other substitution not replacing an amino acid with a like amino acid.

### Cells Engineered for the Production of Olivetolic Acid or a Derivative Thereof or a Cannabinoid

Host cells engineered for the production of olivetolic acid (OA), a derivative of OA, or a cannabinoid are engineered to include a polyketide synthetase activity that produces the intermediate 3,5,7-trioxododecanoyl-CoA or 3,5,7-trioxododecanoic acid and an olivetolic acid cyclase activity that produces olivetolic acid from 3,5,7-trioxododecanoyl-CoA or 3,5,7-trioxododecanoic acid. In various embodiments, the polyketide synthetase activity can produce a 3,5,7-trioxododecanoyl-CoA or 3,5,7-trioxododecanoic acid derivative (for example, using malonyl-CoA and a precursor acyl-CoA other than hexanoyl-CoA) and the olivetolic acid cyclase activity can produce an olivetolic acid derivative from a 3,5,7-trioxododecanoyl-CoA or 3,5,7-trioxododecanoic acid derivative. As disclosed herein, in various embodiments olivetolic acid or an olivetolic acid derivative can be converted to a cannabinoid by the host cell, where the cannabinoid can have the structure of a naturally-occurring or previously identified cannabinoid or may not have the structure of a naturally-occurring or previously identified cannabinoid. Alternatively, olivetolic acid or an olivetolic acid derivative can be the cellular product which can optionally be converted to a cannabinoid in vitro.

As used herein, the term “cannabinoid” refers to a class of compounds that include both naturally occurring and non-naturally occurring compounds, characterized and uncharacterized, thus the term “cannabinoid” as used herein encompasses cannabinoid derivatives—i.e., derivatives of naturally-occurring or known cannabinoids. Exemplary cannabinoids include cannabidiol (CBD), cannabidiolic acid (CBDA), cannabigerol (CBG), cannabigerolic acid (CBGA), cannabichromene (CBC), cannabichromenic acid (CBCA), Δ9-tetrahydrocannabinol (THC), Δ9-tetrahydrocannabinoic acid (Δ9-THCA), cannabinol (CBN), and cannabinolic acid (CBNA). Additional cannabinoids are provided in the disclosure hereinabove. Also included are derivatives of known, naturally-occurring cannabinoids and cannabinoids disclosed herein, which can be any derivatives, and include but are not limited to derivatives having alkyl chain lengths that are longer or shorter than C6.

An engineered cell for producing OA, an OA derivative, or a cannabinoid, includes an exogenous nucleic acid sequence encoding a polyketide synthase that can condense malonyl-CoA with hexanoyl-CoA and an exogenous nucleic acid sequence encoding an olivetolic acid cyclase. The polyketide synthase can be, for example, a Type III polyketide synthase that sequentially condenses three molecules of malonyl-CoA with one molecule of hexanoyl-CoA to produce the olivetolic acid precursor 3,5,7-trioxododecanoyl-CoA and/or 3,5,7-trioxododecanoic acid. (The product of the Type III PKS, e.g., OLS, may be either or both of 3,5,7-trioxododecanoyl-CoA and 3,5,7-trioxododecanoic acid. Thus, throughout this disclosure, when the product of a PKS or OLS is referred to as 3,5,7-trioxododecanoyl-CoA or a derivative thereof, the compound 3,5,7-trioxododecanoic acid or a derivative thereof is also contemplated.) An exemplary Type III polyketide synthase is the Cannabis sativa Olivetol Synthase (OLS, also referred to as “tetraketide synthase”; SEQ ID NO:1. Also considered for use in the engineered cells provided herein are OLS homologs and variants having at least 50%, at least 55%, at least 60%, at least 65%, at least 70%, at least 75%, at least 80%, at least 85%, at least 90%, at least 95%, at least 96%, at least 97% at least 98%, or at least 99% identity to SEQ ID NO:1 that have the activity of a Type III polyketide synthase that produces 3,5,7-trioxododecanoyl-CoA or 3,5,7-trioxododecanoic acid or a derivative thereof from malonyl-CoA and an acyl-CoA. In various examples a Type III polyketide synthase used in the engineered cells and methods provided herein can condense malonyl-CoA with acyl-CoA precursors other than hexanoyl-CoA, for example, can condense malonyl-CoA with propionyl-CoA, butyryl-CoA, valeryl-CoA, octanoyl-CoA, decanoyl-CoA, lauroyl-CoA, myristoyl-CoA, palmitoyl-CoA, stearoyl-CoA, eicosapentaenoyl-CoA, or docosahexaenoyl-CoA, to result in olivetolic acid derivatives and/or cannabinoid derivatives, for example those with varying alkyl side chains of different lengths.

Olivetolic acid cyclase (OAC) of Cannabis sativa converts 3,5,7-trioxododecanoyl-CoA or 3,5,7-trioxododecanoic acid to olivetolic acid (2,4-dihydroxy-6-pentylbenzoate) in a C2-C7 intramolecular aldol condensation (Gagne et al. (2012) Proc Natl Acad Sci USA 109, 12811-12816). (The substrate of OAC may be either or both of 3,5,7-trioxododecanoyl-CoA and 3,5,7-trioxododecanoic acid. Thus, throughout this disclosure, when the substrate of an OAC is referred to as 3,5,7-trioxododecanoyl-CoA or a derivative thereof, the compound 3,5,7-trioxododecanoic acid or a derivative thereof is also contemplated.) Considered for use in the engineered cells provided herein are OACs of Cannabis and OAC homologs of other species, as well as variants of naturally-occurring OACs having at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to SEQ ID NO:2 (OAC, Cannabis sativa) that have the activity of an OAC.

A nucleic acid sequence encoding an OAC can in some embodiments be fused to a nucleic acid sequence encoding a polyketide synthetase of OLS in an engineered cell as provided herein, such that the OAC activity is fused to the polyketide synthase activity, i.e., a fusion protein is produced in the engineered cell that has both condensing and cyclization activities.

In various aspects host cells are engineered for enhanced production of olivetolic acid (OA) or a derivative thereof or a cannabinoid by engineering various pathways into the host cell that increase precursor supply, improve conversion of a precursor to an intermediate or product, or reduce byproduct formation. Enhancing or improving production of olivetolic acid or a derivative thereof of a cannabinoid can be increasing yield, titer, or rate of production.

Thus, in addition to having an exogenous nucleic acid sequence encoding an OAC and an exogenous nucleic acid sequence encoding a Type III PKS, a host cell engineered for the production of a cannabinoid can be engineered to include any or any combination of: over-expression of an acetyl CoA carboxylase (ACC) or ACC subunit or expression of an ACC or ACC subunit variant; expression or over-expression of at least one enzyme for increasing the cell's malonyl-CoA supply, increased supply of at least one cofactor that functions in an enzyme in a malonyl-CoA synthesis pathway; expression or over-expression of at least one enzyme for producing hexanoyl-CoA; expression or over-expression at least one enzyme for producing geranyldiphosphate (GPP); expression or over-expression at least one prenyltransferase; under-expression (downregulation or gene disruption) of any of at least one thioesterase, at least one fatty acid synthesis enzyme, at least one alcohol dehydrogenase, lactate dehydrogenase, phosphate acetyl transferase, or acetate kinase, or at least one enzyme of a fatty acid degradation pathway. The foregoing list of modifications is nonlimiting; additional modifications and cell culture configurations that include cells engineered for enhanced cannabinoid or cannabinoid precursor production are described and referenced in the disclosure below.

Host cells engineered to express an OAC and a Type III PKS, which uses malonyl-CoA as a substrate, and can further include one or more modifications to increase the malonyl-CoA supply. Host cells can be engineered for example to express or overexpress at least one nucleic acid sequence that encodes a polypeptide that functions in a pathway that generates malonyl-CoA. For example, the engineered cell can be engineered to overexpress an endogenous acetyl-CoA carboxylase or to expiess a non-native or variant acetyl-CoA carboxylase subunit or can be engineered to express or overexpress a nucleic acid sequence encoding at least one naturally-occurring enzyme or an enzyme variant that functions in a pathway for producing malonyl-CoA from malonate, oxaloacetate, or beta-alanine.

Host cells that are engineered for increased malonyl-CoA supply can also include genetic modifications such as, but not limited to, downregulation, including disruption, of genes encoding enzymes that may reduce the supply of acetyl-CoA and/or malonyl-CoA available for OA or cannabinoid production, such as but not limited to alcohol dehydrogenase, lactate dehydrogenase, phosphate acetyl transferase, acetate kinase, succinate dehydrogenase, or citrate synthase. Downregulation of one or more genes encoding fatty acid biosynthesis enzymes (e.g., in prokaryotic hosts FabH, FabB, FabG, FabZ, and/or Fabl). Downregulation or knock-out of endogenous thioesterase genes in the host strain can be beneficial to maintaining optimal levels of precursors such as malonyl-CoA and hexanoyl-CoA.

Coenzyme A (CoA) is a component of the precursors hexanoyl-CoA and malonyl-CoA that are substrates of the Type III polyketide synthase that produces 3,5,7-trioxododecanoyl-CoA, and also of acetyl-CoA, the substrate of acetyl-CoA carboxylase that forms the malonyl-CoA precursor. In addition, some embodiments of the invention, set forth below, provide for lactonase degradation of the PDAL, HTAL or other OA derivative byproduct and formation of a OLS pathway intermediate (3,5-dioxodecanoyldiketide-CoA), triketide-CoA or corresponding polyketide-CoA resulting from a lactonase and the activity of a CoA ligase, where the OLS pathway intermediate diketide-CoA, triketide-CoA or corresponding polyketide-CoA can re-enter metabolic reactions and reduce carbon losses. Increasing the supply of CoA to improve the supply of acetyl-CoA, malonyl-CoA, and hexanoyl-CoA, and, in some embodiments, to increase the production of a OLS pathway intermediate triketide-CoA, can be by upregulation of endogenous genes that produce CoA from pantothenate, and/or by introducing exogenous genes encoding enzymes of the pantothenate to CoA pathway. Strategies for increasing CoA include upregulating endogenous genes that produce CoA from pantothenate, for example, by replacing endogenous promoters for genes encoding enzymes of the CoA biosynthesis pathway (pantothenate kinase (PanK, E. coli gene CoaA) (EC 2.7.1.33), phosphopantothenoylcysteine synthetase (PPCS, E. coli gene CoaB, EC 6.3.2.5), phosphopantothenoylcysteine decarboxylase (PPC-DC, E. coli gene CoaC, EC 4.1.1.36), phosphopantotheine adenylyl transferase (PPAT, E. coli gene CoAD), and dephosphocoenzyme A kinase (DPCK, E. coli gene CoaE)) with stronger promoters, which can optionally be inducible promoters. Alternatively or in addition, one or more exogenous genes encoding one or more enzymes of the pantothenate to coenzyme A pathway can be introduced. In various examples a cell culture that includes host cells engineered to over-express at least one enzyme of a CoA biosynthesis pathway can include a culture medium that includes pantothenate, and can optionally also include cysteine, used in CoA biosynthesis.

Pantothenate kinase (EC 2.7.1.33), the first enzyme of the pantothenate to CoA pathway, catalyzes the reaction ATP+(R)-pantothenate=ADP+(R)-4′-phosphopantothenate. In E. coli, pantothenate kinase is competitively inhibited by CoA itself, as well as by some CoA esters. In some embodiments, a host cell is engineered to include a nucleic acid sequence encoding a pantothenate kinase that is not feedback inhibited by coenzyme A, such as, for example, a Type III pantothenate kinase such as CoaX of Pseudomonas putida (UniProtKB-Q88QQ0; SEQ ID NO:3). Additional examples of CoaX pantothenate kinases that may be used in the engineered cells provided herein include, without limitation the Type III pantothenate kinases shown in Table 1.

TABLE 1
Type III Pantothenate Kinases

| Species | Accession No. | SEQ ID NO |
|---|---|---|
| Pseudomonas putida | UniprotKB Q88QQ0 | 3 |
| Pseudomonas sp. | WP_003093768 | 4 |
| Pseudomonas fragi | WP_010655448 | 5 |
| Pseudomonas putida | WP_062575365 | 6 |
| Pseudomonas stutzeri | WP_025240332 | 7 |
| Pseudomonas oryzihabitans | WP_059313753 | 8 |
| Pseudomonas otitidis | WP_074974301 | 9 |
| Pseudomonas brassicacearum | WP_028237101 | 10 |

In yet other embodiments, 4′-phosphopantetheine can be provided in the cell culture medium of a cell engineered produce olivetolic acid, a derivative thereof, or a cannabinoid. 4′-phosphopantetheine is converted to coenzyme A in the last two steps, not requiring the activity of pantothenate kinase. In these embodiments, a host cell can optionally be engineered to include exogenous genes encoding one or both of PPAT and DPCK or can be engineered to overexpress endogenous genes encoding PPAT and/or DPCK.

In additional embodiments that may optionally be combined with the above strategies to increase the supply of CoA, an engineered cell can include an exogenous nucleic acid sequence encoding a pyruvate dehydrogenase (PDH) or can overexpress an endogenous PDH. In some embodiments the engineered cell comprises an exogenous nucleic acid sequence that encodes a variant of a PDH Lpd subunit that includes a mutation that reduces inhibition of PDH by NADH (A358V in the E. coli PDH Lpd subunit, or a mutation in a corresponding amino acid residue in a PDH subunit of another species). In various embodiments, a cell culture that includes a cell that over-expresses PDH or expresses an exogenous PDH, including a variant PDH, the cell culture medium can include thiamine and can optionally also include biotin.

Further strategies for increasing acetyl-CoA that can optionally be combined with expressing genes encoding enzymes of the pantothenate to CoA pathway include expression of an exogenous gene encoding an acetate-CoA ligase (also referred to as an acetyl-coenzyme A synthetase (ACS), AcCoA synthetase, or acyl-activating enzyme (AEE)) or fatty acyl-CoA synthetase (FAA) that can generate acetyl-CoA in an ATP-dependent reaction. Enzymes of the general class of 6.2.1 are acid-CoA ligases. Enzymes having acetate-CoA ligase (ACS) activity are classified as 6.2.1.1, where AMP is a co-product, or 6.2.1.13, where ADP is a coproduct:

Acetate+CoA+ATP→Acetyl-CoA+AMP+PPi (E.C. 6.2.1.1)

Acetate+CoA+ATP→Acetyl-CoA+ADP+Pi (E.C. 6.2.1.13)

ACSs with native activity on acetate will provide the function of increasing acetyl-CoA supply when cells are either supplied with acetate as a co-feed, or where acetate is produced as a by-product. Other sub-classes of 6.2.1, having their main activity on other acid substrates, may also have substantial activity on acetate, and are viable candidates for providing acetate-CoA ligase activity in the engineered cells provided herein. The ACSs expressed in the host cells can be prokaryotic or eukaryotic. Table 2 provides examples of ACSs that can be expressed in a host strain engineered to produce olivetolic acid, an olivetolic acid, or a cannabinoid.

TABLE 2
Acetate-CoA Ligases

| | Accession Number | |
|---|---|---|
| Species | (UniprotKB) | SEQ ID NO |
| Escherichia coli | Q8FAY8 | 11 |
| Salmonella typhimurium | Q8Z1R0 | 12 |
| Bacillus subtilis | P39062 | 13 |
| Methanobacterium | Q2XNL6 | 14 |
| thermoformicicum | | |
| Arabidopsis thaliana | B9DGD6 | 15 |
| Saccharomyces cerevisiae | Q01574 | 16 |
| Homo sapiens | Q9NR19 | 17 |
| Bos taurus | Q9BEA3 | 18 |
| Mus musculus | Q99NB1 | 19 |

TABLE 3
CoA Ligases

| Species | Accession Number | SEQ ID NO |
|---|---|---|
| Ruegeria pomeroyi | WP_011046428 | 20 |
| Burkholderia | | 21 |
| Ruegeria marina | WP_093035935 | 22 |
| Rhodobacteraceae bacterium | WP_008755673 | 23 |
| KLH11 | | |
| Ruegeria atlantica | WP_058279001 | 24 |
| Thioclava arenosa | WP_096433911 | 25 |
| Rhizobium selenitireducens | WP_115669588 | 26 |

The inventors have demonstrated that the 6-carboxy-hexanoate CoA ligase of Ruegeria pomeroyi (SEQ ID NO:20), although not characterized as an acetate-CoA ligase, is active on acetate as well as hexanoate. Additional enzymes characterized as acyl-CoA ligases that are orthologs of the Ruegeria pomeroyi acyl-CoA ligase (WP_011046428; SEQ ID NO:20) include without limitation the Burkholderia 6-carboxy-hexanoate CoA ligase (SEQ ID NO:21), the acyl-CoA synthetase of Ruegeria marina (WP_093035935; SEQ ID NO:22), the acyl-CoA synthetase of Rhodobacteraceae bacterium KLH11 (WP_008755673; SEQ ID NO:23), the acyl-CoA synthetase of Ruegeria atlantica (WP_058279001; SEQ ID NO:24), the acyl-CoA synthetase of Thioclava arenosa (WP_096433911; SEQ ID NO:25), and the acyl-CoA synthetase of Rhizobium selenitireducens (WP_115669588; SEQ ID NO:26). Provided herein are cells engineered to produce olivetolic acid, an olivetolic acid derivative, or a cannabinoid, where the engineered cells also express an exogenous nucleic acid sequence encoding an acyl-CoA synthetase or ligase, where the acyl-CoA synthetase or ligase has activity on acetate. In some embodiments, the engineered cells express an exogenous nucleic acid sequence encoding an acyl-CoA synthetase or ligase, where the acyl-CoA synthetase or ligase has activity on hexanoate or another fatty acid (including acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids) and also has converts acetate to acetyl-CoA. The acyl-CoA ligase can be a naturally-occurring variant having at least 50%, at least 55%, at least 60%, at least 65%, at least 70%, at least 75%, at least 80%, at least 85%, at least 90%, at least 95%, at least 96%, at least 97% at least 98%, or at least 99% identity thereto. In some embodiments the engineered cells include an exogenous nucleic acid sequence encoding an acyl-CoA ligase or synthetase having at least 50%, at least 55%, at least 60%, at least 65%, at least 70%, at least 75%, at least 80%, at least 85%, at least 90%, at least 95%, at least 96%, at least 97% at least 98%, at least 99%, or 100% identity to the acyl-CoA ligase of Ruegeria pomeroyi (SEQ ID NO:20), Burkholderia (SEQ ID NO:21), Ruegeria marina (SEQ ID NO:22), Rhodobacteraceae bacterium KLH11 (SEQ ID NO:23), Ruegeria atlantica (SEQ ID NO:24), Thioclava arenosa (SEQ ID NO:25), or Rhizobium selenitireducens (WP_115669588; SEQ ID NO:26).

Other CoA synthetases or CoA ligases that may convert acetate to acetyl-CoA that can be expressed in a host cell include, for example, the acetate-CoA ligases of Ralstonia solanacearum (SEQ ID NO:27) and Salmonella enterica (SEQ ID NO:28), butyrate-CoA ligases (e.g., the butyrate-CoA ligase of Candidatus Frankia californiensis (SEQ ID NO:29), acetoacetyl-CoA ligases (e.g., the acetoacetate-CoA synthetase from rat liver (SEQ ID NO:30), and propionate-CoA ligases of, for example, Ralstonia solanacearum (SEQ ID NO:31) and Salmonella enterica (SEQ ID NO:32).

Acetyl-CoA Carboxylase (ACC) catalyzes the committed step in fatty acid biosynthesis in carboxylating acetyl-CoA to produce malonyl-CoA, which is the building block for fatty acid biosynthesis. Malonyl-CoA is also a key substrate for cannabinoid formation, where three malonyl-CoA molecules are successively condensed with a hexanoyl-CoA molecule to form the cannabinoid precursor 3,5,7-trioxododecanoyl-CoA or 3,5,7-trioxododecanoic acid. In eukaryotes, including fungi, mammals, and the cytosol of plant cells, ACC is a single large polypeptide with multiple active domains. In prokaryotes and the chloroplasts of plants and algae, ACC is typically a multisubunit enzyme. The typical prokaryotic ACC has four polypeptide subunits (encoded by accA, accB, accC, and accD in E. coli) that assemble into a complex having three functional subunits: a tetramer made up of two homodimers of the biotin carboxylase subunit (BC, P_417722, encoded by accC, in E. coli), a heterotetramer of two of each of the accA and accD subunits (P_414727 and P_416819) forming the carboxytransferase (CT), and the biotin carboxylase carrier protein (BCCP, encoded by accB, P_417721 in E. coli) (see Broussard et al. (2013) Biochemistry 14:3346-3357). In prokaryotes, biotin protein ligase (BPL, encoded by BirA in E. coli) covalently attaches biotin to the carboxylase subunit; this function is performed by a holocarboxylase synthetase in eukaryotes.

In some embodiments, one or more nucleic acid sequences encoding an ACC or at least one subunit thereof is overexpressed in a host cell engineered for the production of OA, a derivative thereof, or a cannabinoid. In embodiments where the host cell is a prokaryotic cell, all or a subset of the polypeptide subunits can be overexpressed. For example, in E. coli, one, two, three, or four subunits can be overexpressed, for example, by expression of an exogenous nucleic acid sequence encoding one or more of the accA, accB, accC, and accD subunits to increase intracellular malonyl-CoA levels (see Davis et al. (2000) J. Biol. Chem. 275:28593-28598). In some embodiments, accB and accC (encoding the BCCP and BC subunits, respectively, that are linked in an operon in E. coli) are overexpressed in a cell engineered for production of olivetolic acid, an olivetolic acid derivative, or a cannabinoid. In some examples, a prokaryotic host cell can include an exogenous nucleic acid molecule that includes the accB and accC genes (optionally in an operon), optionally under the control of a heterologous promoter (see, for example, US2016/0230164, incorporated by reference herein in its entirety, including Table 2 providing examples of modified-sequence promoters of the E. coli accBC operon). The host cell can optionally also include exogenous nucleic acid sequences encoding the accA and/or accD gene that can also optionally be regulated by heterologous promoters. Without limiting the engineered cells and methods of use to any particular mechanisms, it is considered that the use of heterologous promoters can avoid interactions of endogenous transcriptional regulators that may downregulate expression of endogenous promoters. A eukaryotic host such as but not limited to Saccharomyces can also be engineered to overexpress an ACC gene, for example, using a heterologous promoter (e.g., Runguphan & Keasling (2014) Metabolic Engineering 21:103-113) to increase the malonyl-CoA pool in the cell.

TABLE 4
Acetyl CoA Carboxylases.

| Protein | GenBank ID | GI Number | Organism |
|---|---|---|---|
| ACC1 | CAA96294.1 | 1302498 | Saccharomyces |
| | | | cerevisiae |
| KLLA0F06072g | XP_455355.1 | 50310667 | Kluyveromyces lactis |
| ACC1 | XP_718624.1 | 68474502 | Candida albicans |
| YALI0C11407p | XP_501721.1 | 50548503 | Yarrowia lipolytica |
| ANI_1_1724104 | XP_001395476.1 | 145246454 | Aspergillus niger |
| accA | AAC73296.1 | 1786382 | Escherichia coli |
| accB | AAC76287.1 | 1789653 | Escherichia coli |
| accC | AAC76288.1 | 1789654 | Escherichia coli |
| accD | AAC75376.1 | 1788655 | Escherichia coli |

Further, variants of naturally-occurring ACCs or ACC subunits can be expressed in a host cell engineered to produce OA, an OA derivative, or a cannabinoid. In some examples a eukaryotic host cell is engineered to express a variant ACC whose activity is not downregulated by phosphorylation, such as for example the S659A/S1157A mutant of S. cerevisiae (Shi et al. (2014) mBio 5:e01130-14; Ferreira et al. (2018) Metabolic Eng Commun. 6:22-27) or a variant of an ortholog of the Saccharomyces ACC where the variant has mutations at the corresponding amino acid positions. In further embodiments, variants of prokaryotic ACC subunits can be expressed in an engineered prokaryotic host cell. For example, the amino acid at the position corresponding to amino acid 2 of the accB subunit of a prokaryotic acc can be mutated. As disclosed in US2016/0230164, incorporated by reference herein in its entirety, the E. coli accB has an aspartate at position 2 that can be mutated to another amino acid, such as, for example, asparagine, histidine, isoleucine, threonine, tyrosine, arginine, leucine, glutamine, glycine, or serine. In various embodiments, an orthologous accB (BCCP subunit) can be mutated at position 2, or where position 2 of the orthologous accB is not aspartate or glutamate, at the most proximal aspartate or glutamate residue. Additional variants that can be expressed in a prokaryotic host such as E. coli to increase the malonyl-CoA supply include those listed in Table 3 of US2016/0230164.

Further genetic modifications to increase ACC activity by reducing endogenous regulation of prokaryotic ACC genes are also considered. For example, “refactoring” the accA and accD genes such that their nucleic acid sequences, and the nucleic acid sequences of their RNA transcripts, are altered, can be employed to prevent binding of the CT (accA, accD) subunit polypeptide to the accA and/or accD transcripts, which can downregulate translation of accA and accD polypeptides (Meades et al. (2010) Nucl. Acids Res. 38:1217-1227). The refactored genes can encode a wild type accA and/or accD polypeptide, or variants thereof. Alternatively or in addition, GlnB a gene encoding a regulator of the transcription of multiple genes in E. coli (PII), can be downregulated in an engineered host cell as provided herein to reduce negative regulation of acc transcription.

Malonyl-CoA can also be increase by engineering pathways into the host cell that do not rely on ACC. For example, a cell engineered for the production of a cannabinoid as provided herein can include an exogenous gene encoding a malonyl-CoA synthetase, a malonate transporter, a methylmalonyl-CoA carboxytransferase, a beta-alanine pyruvate acetyltransferase, or a malonyl-CoA reductase. In various embodiments, an engineered cell that expresses one or more of these enzymes produces more of a cannabinoid, olivetolic acid, an olivetolic acid derivative, than is produced by a substantially equivalent cell that does not express one or more of a malonyl-CoA synthetase, a malonate transporter, a methylmalonyl-CoA carboxytransferase, a beta-alanine pyruvate acetyltransferase, or a malonyl-CoA reductase.

For example, in some embodiments a recombinant cell engineered for the production of olivetolic acid, a derivative of olivetolic acid, or a cannabinoid can include an exogenous nucleic acid sequence encoding a malonyl-CoA synthetase (EC 6.2.1.14). Malonyl-CoA synthetase catalyzes the ATP-dependent reaction: malonic acid+CoASH→malonyl-CoA. Nonlimiting examples of malonyl-CoA synthetases that can be expressed in an engineered host cell as provided herein include those encoded by the matB gene of Rhizobium leguminosarum (SEQ ID NO:33), Rhizobiales bacterium (SEQ ID NO:34), and Streptomyces coelicolor (AL163003; SEQ ID NO:36) as well as the malonyl-CoA synthetase encoded by the AAE13 gene of Arabidopsis (At3g16170; SEQ ID NO:39), and other homologs listed in Table 5, variants having at least 50%, at least 55%, at least 60%, at least 65%, at least 70%, at least 75%, at least 80%, at least 85%, at, least 90%, at least 95%, at least 96%, at least 97% at least 98%, or at least 99% identity to the referenced sequences or to naturally-occurring orthologs of the referenced malonyl-CoA synthetases. Hughes & Keatings-Clay (2011) Chemistry & Biology 18:165-176 provide a crystal structure of the S. coelicolor malonyl-CoA synthetase and also provide Table 2 that includes conserved motifs found in malonyl-CoA synthetases and other CoA-thioester-forming enzymes. The matB gene of bacterial species occurs in a matABC operon, where matA is a malonyl-CoA decarboxylase, matB is the malonyl-CoA synthetase, and matC is a malonate transporter. A host cell engineered to produce a cannabinoid, olivetolic acid, or a derivative of olivetolic acid can, in addition to expressing a gene encoding a malonyl-CoA synthetase, further include a gene encoding a malonate transporter, e.g., a transporter encoded by a naturally-occurring matC gene (such as but not limited to a matC gene of a Rhizobium or Streptomyces species or for example a transporter listed in Table 6), or encoding a malonyl-CoA transporter having at least 50%, at least 55%, at least 60%, at least 65%, at least 70%, at least 75%, at least 80%, at least 85%, at least 90%, at least 95%, at least 96%, at least 97% at least 98%, or at least 99% identity to a naturally-occurring malonate transporter. In addition to malonate transporters encoded by matC genes, malonate transporters can include those encoded by the mdcF gene of Klebsiella pneumoniae (Hoenke et al. (1997) FEBS J. 246:530-538), homologs of the mdcF transporter in other species, and variants of naturally-encoded malonate transporters of the mdcF family having at least 50%, at least 55%, at least 60%, at least 65%, at least 70%, at least 75%, at least 80%, at least 85%, at least 90%, at least 95%, at least 96%, at least 97% at least 98%, or at least 99% identity to a naturally-occurring malonate transporter. Additional examples of malonate transporters in prokaryotic species include those having two subunits encoded by madL and madM genes in Malonomonas rubra and Pseudomonas putida (Schaffitzel et al. (1998) J. Bacteriol. 180:2689). Cell cultures of a host cell engineered to express a malonyl-CoA synthetase and a malonate transporter can include a culture medium that includes malonate.

TABLE 5
Malonyl-CoA Synthetases

| | Accession | SEQ ID |
|---|---|---|
| Species | Number | NO |
| Rhizobium leguminosarum | AAC83455 | 33 |
| Rhizobiales bacterium | WP_115097553 | 34 |
| Rhizobium phaseoli | WP_097602133 | 35 |
| Streptomyces coelicolor | 3NYR | 36 |
| Streptomyces canus | WP_059299214 | 37 |
| Streptomyces luteus | WP_043373306 | 38 |
| Arabidopsis thaliana | Q8H151 | 39 |
| Camelina sativa | XP_010465627 | 40 |
| Brassica rapa | XP_009146164 | 41 |

TABLE 6
Putative Malonate Transporters

| | Accession | SEQ ID |
|---|---|---|
| Species | Number | NO |
| Rhizobium leguminosarum | AAC83457 | 42 |
| Rhizobium phaseoli | WP_064817596 | 43 |
| Streptomyces coelicolor | CAB86108 | 44 |
| Streptomyces pactum | WP_055416767 | 45 |

In additional embodiments, a cell engineered for the production of olivetolic acid or a derivative thereof or a cannabinoid that includes a polyketide synthase that condenses malonyl-CoA with an acyl-CoA primer and an olivetolic cyclase that forms olivetolic acid or a derivative thereof is further engineered to include an exogenous nucleic acid sequence that encodes a methylmalonyl-CoA carboxytransferase that can transfer a carboxyl group from oxaloacetate to acetyl-CoA to generate malonyl-CoA. An engineered cell that expresses an exogenous nucleic acid sequence encoding a methylmalonyl-CoA carboxytransferase can also optionally be engineered to express or overexpress a nucleic acid sequence that encodes a polypeptide whose activity increases production of oxaloacetate, such as for example an aspartate transaminase (EC 2.6.1.1). Aspartate transaminase catalyzes the interconversion of aspartate and alpha-ketoglutarate to oxaloacetate and glutamate, and a gene encoding an aspartate transaminase introduced into a host cell engineered for cannabinoid synthesis can encode an aspartate transaminase endogenous to the host cell, providing the gene in higher copy number and/or with a promoter that expresses the enzyme at a higher level. Alternatively, an endogenous aspartate transaminase gene can be overexpressed by replacement or mutation of its native promoter. In further alternatives for overexpressing aspartate transaminase, a gene introduced into a host cell as provided herein can encode an aspartate transaminase of a different species than the host cell, or can encode a variant of a naturally-occurring aspartate transaminase. A cell culture that includes a cell engineered for the production of olivetolic acid or a cannabinoid or a derivative of either and that expresses a nucleic acid sequence encoding a methylmalonyl-CoA carboxytransferase can in some embodiments be cultured in a medium that includes one or a combination of oxaloacetate, aspartate, and glutamate.

In another embodiment, a host cell engineered to produce a cannabinoid or olivetolic acid or a derivative thereof can be engineered to express a methylmalonyl-CoA carboxytransferase, such as, but not limited to an MMC of Propionibacterium freudenreichii (1.3S, 5S and 12S subunits provided as SEQ ID NOS:46-48), C. thermocellum (1.3S, 5S and 12S subunits provided as SEQ ID NOS:49-51), T. saccharolyticum (1.3S, 5S and 12S subunits provided as SEQ ID NOS:52-54), Caldicellulosiruptor beschii (1.3S, 5S and 12S subunits provided as SEQ ID NOS:55-57), Corynebacterium kroppenstedtii (1.3S, 5S and 12S subunits provided as SEQ ID NOS:58-60), and Geobacter bemidjiensis (1.3S, 5S and 12S subunits provided as SEQ ID NOS:61-63). A host cell can also be engineered to express a naturally-occurring ortholog of these MMCs, or variants having at least 50%, at least 55%, at least 60%, at least 65%, at least 70%, at least 75%, at least 80%, at least 85%, at least 90%, at least 95%, at least 96%, at least 97% at least 98%, or at least 99% identity to the referenced gene or their orthologs in other species. An engineered host cell that includes a non-native gene encoding an MMC can optionally further be engineered to express of overexpress a nucleic acid sequence encoding an aspartate transaminase. A cell culture that comprises cells engineered to express an exogenous gene encoding an MMC and, optionally, an aspartate transaminase, can optionally include one or more of oxaloacetate, aspartate, or glutamate in the culture medium.

In a further embodiment, a host cell engineered to produce a cannabinoid or olivetolic acid or a derivative thereof can be engineered to include a pathway from beta-alanine to malonyl-CoA. An engineered cell that includes a beta-alanine to malonyl-CoA pathway can include a gene encoding a beta-alanine pyruvate aminotransferase (BAPAT). BAPATs include, but are not limited to, a BAPAT of Table 7, naturally-occurring orthologs of these BAPATs, or variants having at least 50%, at least 55%, at least 60%, at least 65%, at least 70%, at least 75%, at least 80%, at least 85%, at least 90%, at least 95%, at least 96%, at least 97% at least 98%, or at least 99% identity to the referenced gene or their orthologs in other species. An engineered host cell that includes a non-native gene encoding a BAPAT can optionally further be engineered to express of overexpress a nucleic acid sequence encoding a malonyl-CoA reductase (MCR) that reduces malonyl semialdehyde generated by a BAPAT to malonyl-CoA. A cell culture that comprises cells engineered to express an exogenous gene encoding a BAPAT and, optionally, an MCR, can optionally include one or both of oxaloacetate, aspartate, or beta-alanine in the culture medium.

TABLE 7
Beta-alanine pyruvate aminotransferases

| | Accession | SEQ ID |
|---|---|---|
| Species | Number | NO |
| Pseudomonas putida | P28269 | 64 |
| Pseudomonas sp. | A0A1G4TV26 | 65 |
| Pseudomonas sp. | K9NF79 | 66 |
| Pseudomonas sp. | W6VT41 | 67 |
| Pseudomonas sp. | A0A1I5LP76 | 68 |
| Pseudomonas chlororaphis | J2YJG4 | 69 |
| Pseudomonas moorei | A0A1H1JBF4 | 70 |
| Pseudomonas synxantha | I4KU52 | 71 |
| Pseudomonas chlororaphis | A0A1E1FQF6 | 72 |
| Pseudomonas asplenii | A0A1H1Q5W6 | 73 |

The oxidation of malonyl semialdehyde to malonyl-CoA by MCR generates NADPH from NADP+. To ensure a supply of NADP+, a host cell engineered to express an exogenous BAPAT and an exogenous MCR, and the cell engineered to produce a cannabinoid, olivetolic acid, or an olivetolic acid precursor can be further engineered to express an NADPH oxidase that converts NADPH to NADP+ in the reaction: 2NADPH+2H++O₂→2NADP++2H₂O. The NADPH oxidase can be, for example, a water-forming NADPH oxidase that is derived from an NADH oxidase by mutation of residues in the dinucleotide binding motif (GxGxxG/A) and the substrate specificity loop. Such regions can be identified in other NAD oxidoreductases for example by aligning the sequences for maximum homology or motif analysis. For example, the TPNOX enzyme disclosed in Cracan et al. (2017) Nat. Chem. Biol. (SEQ ID NO:74) has the mutations G159A, D177A, A178R, M179S, P184R with respect to the wild type sequence from which it is derived (Lactobacillus brevis, Q8KRG4, SEQ ID NO:106). Nonlimiting examples of NAD oxidoreductases that can be mutated at corresponding amino acid positions include, without limitation, those of SEQ ID Nos:74-85.

Hexanoyl-CoA is condensed with three molecules of malonyl-CoA by the Type III PKS that generates the olivetolic acid precursor 3,5,7-trioxododecanoyl-CoA or 3,5,7-trioxododecanoic acid. In some embodiments, hexanoic acid is produced by fatty acid biosynthesis and the activity of a thioesterase having activity on hexanoyl-ACP. (The terms for a fatty acid and its conjugate base are used interchangeably herein, for example, where hexanoic acid is stated, hexanoate is also intended, and vice versa.) One or more enzymes of the fatty acid biosynthesis pathway, such as, in an E. coli host, a beta-ketoacyl-ACP synthases FabH (P 415609) and FabB (NP 416826), acetoacetyl-ACP reductase FabG (P 415611), 3-hydroxyacyl-ACP dehydratase FabZ (P_414722) and enoyl-ACP reductase Fabl (P_415804), can optionally be upregulated for enhanced production of hexanoyl-CoA. In some embodiments, an exogenous thioesterase, e.g, an acyl-ACP thioesterase, having a short acyl chain substrate preference, can be expressed in the host cell. Alternatively, an endogenous thioesterase can be overexpressed in the host cell, such as, for example, TesA, or a variant thereof (see, for example, U.S. Pat. No. 9,175,234), including an N-terminally truncated variant thereof (e.g., ‘TesA (Cho & Cronan (1993) J. Biol. Chem. 268:9238-9245), or an ortholog of TesA or a variant of a TesA ortholog (including an N-terminally truncated ortholog of TesA). Further nonlimiting examples of short chain thioesterases that can be expressed in an engineered host strain to generate hexanoic acid include, without limitation, the acyl-ACP thioesterase of Bacteroides fragilis (WP_005797964), Bacteroides thetaiotaomicron, Bryantella formatexigen, Lactobacillus brevis, Lactobacillus plantarum, and Streptococcus dysgalactiae as provided in WO2018/200888 as SEQ ID NO:27-31, orthologs of these thioesterases, and variants of these thioesterases and their naturally-occurring orthologs having at least 50%, at least 55%, at least 60%, at least 65%, at least 70%, at least 75%, at least 80%, at least 85%, at least 90%, at least 95%, at least 96%, at least 97% at least 98%, or at least 99% identity to the references thioesterases, including variants that are N-terminally truncated to remove the leader sequence.

In some embodiments, the host cells are engineered to produce an olivetolic acid derivative, e.g., a 2-alkyl-4,6-dihydroxybenzoic acid having an alkyl chain length other than C6, that can be a precursor to a cannabinoid having an alkyl chain length other than C6. In various examples of these embodiments, the host cells can be engineered to express a thioesterase, e.g., an acyl-ACP thioesterase, having an acyl chain length substrate preference that is other that a C6 acyl chain length preference. For example, a recombinant cell engineered to produce OA, an OA derivative, or a cannabinoid can be engineered to include an exogenous nucleic acid sequence encoding a thioesterase having, as nonlimiting examples, a C10, C12, or C14 substrate preference.

Thioesterases include thioester hydrolases, which are identified as E.C. 3.1.2 and are obtainable from a variety of sources. Plant thioesterases are described in, for example, Voelker and Davies, J. Bact., Vol., 176:7320-27, 1994, U.S. Pat. Nos. 5,667,997, and 5,455,167. Thioesterases are also obtainable from microbial sources, such as those described in Akoh et al., Prog. Lipid Res., vol. 43:534-52, 2004; Diczfalusy and Alexson, Arch. Biochem. Biophys., vol. 334:104-12, 1996; Larson and Kolalttukudy, Arch. Biochem. Biophys., vol. 237:27-37, 1985; Lawson et al., Biochemistry, vol. 33:9382-88, 1994; Lee et al., Eur. J. Biochem., vol. 184:21-28, 1989; Naggert et al., J. Biol. Chem., vol. 266:11044-50, 1991; Nie et al., Biochemistry, vol. 47:7744-51, 2008; Seay and Lueking, Biochemistry, vol. 25:2480-85, 1986; Spencer et al., J. Biol. Chem., vol. 253:5922-26, 1978; and Zhuang et al., Biochemistry, vol. 47:2789-96, 2008. Thioesterases are also obtainable from, for example, cyanobacterial, algal, mammalian, insect, and fungal sources.

In some embodiments, hexanoic acid or another fatty acid (including propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids) can be produced by an engineered reverse beta oxidation pathway, as described, for example in U.S. Pat. No. 9,416,364, WO 2017/161041, and WO2018/200888, all of which are herein incorporated by reference in their entireties. A reverse beta oxidation pathway can include, for example, a ketoacyl-CoA thiolase (e.g., BktB (AAC38322) from R. eutropha), a 3-hydroxyacyl-CoA dehydrogenase and enoyl-CoA hydratase multifunctional enzyme (e.g., FadB from E. coli (P 418288)) and an enoyl-CoA reductase (e.g., EgTer from E. gracilis (Q5EU90)), and optionally a thioesterase for terminating the acyl chain. In some embodiments a thioesterase expressed in the engineered host cell is a truncated TesA thioesterase or a variant thereof (see, for example, U.S. Pat. No. 9,175,234). In some examples an engineered host strain can include an exogenous nucleic acid sequence encoding a thioesterase having an acyl chain length substrate preference corresponding to the desired alkyl chain length for an olivetolic acid derivative and a cannabinoid produced therefrom.

In yet other embodiments, hexanoate, or another acid of a desired acyl chain length, can be provided in the culture medium. In various examples of these embodiments, the cell is not engineered to express an exogenous acyl-ACP thioesterase or over-express an endogenous acyl-ACP thioesterase. In embodiments where a fatty acid is provided in the culture medium to supply a substrate for the PKS enzyme that forms 3,5,7-trioxododecanoyl-CoA, 3,5,7-trioxododecanoic acid, or a derivative thereof, it can be advantageous to downregulate or delete one or more thioesterase genes that are endogenous to the host strain. Without limiting the invention to any particular mechanism, reducing thioesterase activity in the cell may prevent degradation of a precursor acyl-CoA or malonyl-CoA in forming olivetolic acid or a derivative thereof (such as, for example, a 2-alkyl-4,6-dihydroxybenzoic acid having an alkyl chain length that is not C6).

In various embodiments, where hexanoate/hexanoic acid or another organic acid is produced by the host cell by any pathway, or where hexanoate/hexanoic acid or another organic acid acid, such as but not limited to, butanoic acid (butyric acid), pentanoic acid (valeric acid), hexanoic acid (caproic acid), heptanoic acid (enanthic acid), or octanoic acid (caprylic acid), is supplied in the culture medium, the engineered host cell can overexpress an endogenous CoA ligase with activity on the produced or provided organic acid, or the host cell can be engineered to include an exogenous nucleic acid sequence encoding a CoA ligase, an acyl-activating enzyme (AAE), or a fatty acyl-CoA synthetase (FAA) with activity on the produced or provided fatty acid. In some examples, the acyl-CoA ligase, AAE, or FAA converts hexanoate to hexanoyl-CoA. In some examples, the exogenous CoA ligase, AAE, or FAA generates acyl-CoA from a fatty acid (including propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids), more preferably from propionate, butyrate, valerate, isovalerate, hexanoate, heptanoate, octanoate, decanoate, or dodecanoate. In various embodiments acyl-CoA ligases expressed in the engineered cells provided herein can use fatty acids of varying chain lengths as well acetate and/or malonate as substrates.

In some examples, the engineered host cell is engineered to include a nucleic acid sequence encoding an acyl-activating enzyme from Cannabis sativa that has activity on hexanoate such as or CsAAE3 (SEQ ID NO:26) or CsAAE1 (SEQ ID NO:27), or an ortholog of either, or a variant of CsAAE3 or CsAAE1, an ortholog of CsAAE3 or CsAAE1, or a variant of any of CsAAE3, CsAAE1, or their orthologs having at least 50%, at least 55%, at least 60%, at least 65%, at least 70%, at least 75%, at least 80%, at least 85%, at least 90%, at least 95%, at least 96%, at least 97% at least 98%, or at least 99% identity to SEQ ID NO:26 or SEQ ID NO:27. In additional embodiments, the CoA ligase expressed in the host cell is the RevS acyl-CoA synthetase of Streptomyces (SEQ ID NO:28), an ortholog thereof, or a variant thereof having at least 50%, at least 55%, at least 60%, at least 65%, at least 70%, at least 75%, at least 80%, at least 85%, at least 90%, at least 95%, at least 96%, at least 97% at least 98%, or at least 99% identity to SEQ ID NO:28.

Additional examples of CoA synthetases that can be expressed in a host cell engineered to produce a cannabinoid or olivetolic acid or a precursor thereof include, without limitation, the acetyl-CoA synthetase encoded by the acs gene of E. coli (UniprotKB Q8FAY8, SEQ ID NO:), the ACS of Salmonella typhimurium (UniprotKB Q8Z1R0, SEQ ID NO:), orthologs of these ACSs in other species, and variants of naturally-occurring ACSs having at least 50% thereto. Other CoA ligases having activity on hexanoate as well as acetate include, for example, the Ruegeria pomeroyi 6-carboxy-hexanoate CoA ligase (SEQ ID NO:) and the 6-carboxy-hexanoate CoA ligase from Burkholderia (SEQ ID NO:21). Candidate CoA ligases for activating acids that may be condensed with malonyl-CoA to provide olivetolic acid precurosors (e.g., 2-alkyl-4,6-dihydroxybenzoic acids) include the butyrate-CoA ligases of Pseudomonas (CDM46008, SEQ ID NO:92), Streptomyces (SEQ ID NO:93), Salmonella (SEQ ID NO:28), Candidatus (SEQ ID NO:29), and Homo sapiens (SEQ ID NO:94) and orthologs and variants thereof, the acetoacetate-CoA ligase isolated from rat liver (UniprotKB Q9JMI1, SEQ ID NO:30), and the propanoate-CoA ligases of Ralstonia solanacearum (SEQ ID NO:31) and Salmonella choleraesuis (SEQ ID NO:32). Considered for use in engineered cells that produce a cannabinoid, olivetolic acid, or a derivative of olivetolic acid are the referenced CoA ligases, as well as their orthologs in other species and variants of the referenced CoA ligases and their orthologs having at least 50%, at least 55%, at least 60%, at least 65%, at least 70%, at least 75%, at least 80%, at least 85%, at least 90%, at least 95%, at least 96%, at least 97% at least 98%, or at least 99% identity to the naturally-occurring enzymes.

In some embodiments provided herein, hexanol is supplied to the host cells in the culture medium and the host cell that is engineered to produce OA, a derivative of OA, or a cannabinoid, is engineered to express or overexpress an alcohol dehydrogenase that converts hexanol to hexanal and an aldehyde dehydrogenase (ALD) that converts the aldehyde to the fatty acyl-CoA. Some enzymes referred to as alcohol dehydrogenases are known to have both activities. For example, a host cell engineered for the production of a cannabinoid can also be engineered to overexpress a native alcohol dehydrogenase and a native aldehyde dehydrogenase, or the dual-acting enzyme. Where the host cell is E. coli or another bacterial species, the alcohol dehydrogenase can be adhE or an ortholog thereof in another species, or an alternative alcohol dehydrogenase that may be endogenous to the bacterial host. In Saccharomyces, the alcohol dehydrogenase may be any of ADH1, ADH2, ADH3, ADH4, ADH5, ADH6, or ADH7. Also considered is the AdhE2 from Clostridium acetobutylicum (Q9ANR5). Adh genes are well-known and one or more ADH genes of a selected host strain can be selected for overexpression. Ald genes are well-known and one or more ALD genes of a selected host strain can be selected for overexpression. Dual-acting enzymes are also well known that can be selected for overexpression.

In further embodiments, a host strain may be engineered to express an exogenous alcohol dehydrogenase and/or ALD, which can be from any source, including mammals, insects, plants, yeast, or bacteria. In these embodiments the engineered host can further be engineered to express or overexpress a CoA ligase that has activity on the fatty acid produced from the fatty aldehyde as a byproduct, such as but not limited to any described above.

Alternatively or in addition to one or more modifications for producing malonyl-CoA and/or one or more modifications for producing hexanoyl-CoA, a cell engineered for the production of olivetolic acid, a cannabinoid, or derivative of olivetolic acid or a cannabinoid can further be engineered downregulate or disrupt a gene encoding a transporter that can transport PDAL molecules out of the cell. Candidate transporters include but are not limited to, any of the mexA-mexB-oprM transporters of prokaryotic hosts. Without limiting the invention to any particular mechanisms, it is contemplated that preventing or reducing the movement of the PDAL byproduct out of the cell can allow for recycling of the PDAL molecule's carbon within the cell.

In further embodiments, one or more lactonases that can hydrolyze the byproduct PDAL can be expressed in the cell. Considered as lactonases that can be expressed in an engineered cell as provided herein are, as nonlimiting examples, acyl-homoserine lactonases, 1,4-lactonases, 2-pyrone-4,6-dicarboxylate lactonases, 3-oxoadipate enol-lactonases, actinomycin lactonases, deoxylimonate A-ring lactonases, gluconolactonases, L-rhamnono-1,4-lactonases, limonin-D-ring-lactonases, steroid lactonases, triacetate-lactonases, and xylono-1,4-lactonases. In some embodiments, a lactonase that can be expressed in an engineered cell as provided herein can be an acyl-homoserine lactonase. In other embodiments, a lactonase that can be expressed in an engineered cell as provided herein can be a 2-pyrone-4,6-dicarboxylate lactonase (EC 3.1.1.38). In further embodiments, a lactonase expressed in an engineered cell as provided herein can be a triacetate-lactonase (EC 3.1.1.57). Without limiting the invention to any particular mechanisms, it is contemplated that hydrolyzing a PDAL molecule can allow for further metabolism of the hydrolyzed molecule, including in pathways that lead to cannabinoids. A lactonase can be expressed in a host cell that is genetically modified to have reduced expression of a transporter that may allow for release of PDAL from the cell. In such embodiments, carbon loss to the PDAL byproduct can be reduced by retaining the carbon molecule in the cell and degrading it so that the carbons can be recycled.

In another aspect, cells engineered to produce a cannabinoid, olivetolic acid or a derivative of olivetolic acid, are further engineered to reduce the accumulation of the byproduct pentyl diacetic acid lactone (PDAL), hexanoyl triacetic acid lactone (HTAL) or the corresponding byproduct of the OA derivative. In some embodiments, the host cells are engineered to disrupt or delete one or more endogenous genes encoding lactone transporters that may transport PDAL, HTAL or the corresponding byproduct molecules out of the cell. In some embodiments, the host cells are engineered to produce a lactonase that hydrolyzes the lactone ring of PDAL, HTAL, or the corresponding byproduct to generate a diketide, triketide or corresponding polyketide hydrolysis product respectively, as described above. In various examples of this embodiments the host cell also expresses a CoA ligase that converts the hydrolyzed PDAL, HTAL or corresponding byproduct molecule to a diketide-CoA, a triketide-CoA or corresponding polyketide-CoA. Examples of CoA ligases that may be useful in converting a di- or triketide or corresponding polyketide to a di- or triketide-CoA or corresponding polyketide-CoA, respectively, include, but are not limited to, some of the CoA ligases disclosed herein for generating hexanoyl-CoA, including, for example the Ruegeria pomeroyi 6-carboxy-hexanoate CoA ligase (SEQ ID NO:20) and the 6-carboxy-hexanoate CoA ligase from Burkholderia (SEQ ID NO:21) or any of the CoA ligases listed in Table 1, and orthologs and variants thereof. Without limiting the invention to any particular mechanism, in some embodiments the CoA-thioesterified HTAL degradation product (triketide) or the corresponding Co-A-esterified polyketide can be acted on by biosynthetic enzymes such as a polyketide synthase and optionally an olivetolic acid cyclase to generate olivetolic acid or desired derivative of olivetolic acid, thus recycling carbons that would otherwise be lost in an excreted byproduct. In the case of PDAL, the resulting CoA-thioesterified diketide product can be condensed with a third molecule of malonyl-CoA to generate a triketide-CoA, which can be acted on by biosynthetic enzymes such as a polyketide synthase and optionally an olivetolic acid cyclase to generate olivetolic acid, again recycling carbons that would otherwise be lost in an excreted byproduct.

In further embodiments, a cell engineered for the production of a cannabinoid or cannabinoid derivative that expresses an exogenous Type III PKS in combination with an exogenous olivetolic acid synthase can be further engineered to include an exogenous nucleic acid sequence encoding at least one enzyme that participates in the conversion of prenol or isoprenol to GPP as is known to occur through a set of reactions. As described in detail below, prenol is phosphorylated to DMAP and isoprenol to IP. DMAP is phosphorylated to DMAPP and IP to IPP. DMAPP and IPP can be interconverted via an isomerase. DMAPP and IPP are reacted together to produce GPP. For example, a cell engineered to produce a cannabinoid can include a nucleic acid sequence encoding an alcohol kinase, such as, for example, the E. coli alcohol kinase YchB (NP_415726; SEQ ID NO:100) or the Thermoplasma acidophilum phosphate kinase ThaIPK having the mutations V73I, Y141V, and K204G that increase activity on prenol or isoprenol (WP_010900530; SEQ ID NO:101) or orthologs thereof, or variants of these kinases and their orthologs having at least 50%, at least 55%, at least 60%, at least 65%, at least 70%, at least 75%, at least 80%, at least 85%, at least 90%, at least 95%, at least 96%, at least 97% at least 98%, or at least 99% identity thereto that convert prenol or isoprenol to dimethylallyl phosphate (DMAP) or isopentenyl phosphate (IP), respectively. The engineered cells can further include an exogenous nucleic acid sequence encoding a prenyl phosphate kinase (or isopentenyl phosphate kinase, IPK) that can convert DMAP or IP to dimethylallyl pyrophosphate (DMAPP) or isopentenyl pyrophosphate, such as, for example, the Methanothermobacter thermautotrophicus prenyl phosphate kinase MtIPF (AAB84554), orthologs thereof, or variants having at least 50% identity to naturally-occurring phosphate kinases. Additionally, an engineered cell can include an exogenous nucleic acid sequence encoding an alcohol diphosphoisomerase such as the E. coli isopentenyl pyrophosphate isomerase (Q46822; SEQ ID NO:107) that converts DMAPP to IPP. The cells engineered for cannabinoid production can further include a GPP synthase, such as E. coli IspA (NP_414955, SEQ ID NO: 102) or a GPP synthase from Abies Grandis (GPPS2; AAN01134, with 84 amino acid N-terminal truncation, SEQ ID NO:104). GPP synthases that are orthologs of these examples of GPP synthases or are variants of these GPP synthases or their orthologs having at least 50%, at least 55%, at least 60%, at least 65%, at least 70%, at least 75%, at least 80%, at least 85%, at least 90%, at least 95%, at least 96%, at least 97% at least 98%, or at least 99% amino acid sequence identity thereto are also considered for use in a host cell as provided herein.

In other examples, a cell engineered to produce a cannabinoid can include a nucleic acid sequence encoding an alcohol kinase that converts geraniol to geranyl monophosphate and can further include a nucleic acid sequence encoding a geranyl phosphate kinase (GPK) that converts geranyl monophosphate to GPP. The farnesol kinase (FOLK) of Arabidopsis thaliana (UniprotKB Q67ZM7; SEQ ID NO:105) is an example of a geraniol kinase that can be expressed in an engineered host cell for conversion of geraniol to geranyl monophosphate. Also considered are farnesol kinases that are orthologs of the Arabidopsis farnesol kinase and have geraniol kinase activity, as well as variants having at least 50% identity thereto. Examples of geranyl phosphate kinases that can be expressed in a cell engineered for the production of cannabinoids include the isopentenyl phosphate kinase of Thermoplasma acidophilum with mutated amino acids at positions 70 and 140, optionally in combination with mutations at amino acid positions 73 and/or 130, and the isopentenyl phosphate kinase of Methanothermobacter thermoautotrophicus with mutated amino acids at positions 77, 142, and 154. Also considered are orthologs of the Thermoplasma and Methanothermobacter isopentenyl phosphate kinases, including variants of orthologs with mutations at the corresponding positions, as well as isopentenyl phosphate kinases having at least 50%, at least 55%, at least 60%, at least 65%, at least 70%, at least 75%, at least 80%, at least 85%, at least 90%, at least 95%, at least 96%, at least 97% at least 98%, or at least 99% identity to the Thermoplasma and Methanothermobacter isopentenyl phosphate kinases.

A cell engineered to produce a cannabinoid or a cannabinoid derivative can also be modified to disrupt or delete endogenous genes that encode hydrolases that can dephosphorylate geranyldiphosphate or geranyldiphosphate precursors such as IPP and DMPP. For example, in E. coli the nudix hydrolase genes NudB, NudF, Nudl, and NudJ (EC 3.6.1) can be downregulated or disrupted, and in Saccharomyces cerevisiae, the Ysa1p gene can be downregulated or disrupted. Orthologous genes in other host species can be similarly downregulated or disrupted. Alkaline phosphatase genes of E. coli, such as, for example, PhoA, AphA, BacA, PgpA, and/or PgpB can also be deleted, disrupted, or downregulated. Where the engineered host cell is a eukaryotic cell such as a fungal cell, a diacylglycerol diphosphate phosphatase gene or a lipid phosphate phosphatase gene such as DPP1 and LPP1, respectively, in Saccharomyces cerevisiae or orthologous genes in other eukaryotes can be downregulated or disrupted to prevent hydrolysis of GPP or its precursors.

A host cell engineered for the production of a cannabinoid can include a prenyltransferase that uses GPP to prenylate olivetolic acid resulting in the production of cannabigerolic acid (CBGA). Examples of prenyltransferases that can be expressed in a host cell include, without limitation, the C. sativa geranyl-pyrophosphate-olivetolic acid geranyltransferase (GOT, EC 2.5.1.102) and orthologs thereof, as well as variants of the GOT of C. sativa and orthologs thereof having at least 50%, at least 55%, at least 60%, at least 65%, at least 70%, at least 75%, at least 80%, at least 85%, at least 90%, at least 95%, at least 96%, at least 97% at least 98%, or at least 99% identity to a naturally-occurring geranyl-pyrophosphate-olivetolic acid geranyltransferase (see for example, WO 2018/200888, incorporated herein by reference). Also considered for expression in host cells engineered for producing cannabinoids are aromatic prenyltransferases of bacterial species such as the Streptomyces NphB gene product and variants thereof having at least 50%, at least 55%, at least 60%, at least 65%, at least 70%, at least 75%, at least 80%, at least 85%, at least 90%, at least 95%, at least 96%, at least 97% at least 98%, or at least 99% identity to a naturally-occurring aromatic prenyltransferase.

A host cell engineered for the production of a cannabinoid can optionally include an exogenous gene encoding a cannabinoid synthase that can be used to produce particular cannabinoids. Nonlimiting examples of cannabinoid synthases include a C. sativa tetrahydrocannabinolic acid (THCA) synthase polypeptide and a C. sativa cannabidiolic acid (CBDA) synthase polypeptide as well as derivatives thereof having at least 50%, at least 55%, at least 60%, at least 65%, at least 70%, at least 75%, at least 80%, at least 85%, at least 90%, at least 95%, at least 96%, at least 97% at least 98%, or at least 99% identity to naturally-occurring cannabinoid synthases.

Additional genetic modifications that may be present in a host cell engineered to produce olivetolic acid or a derivative thereof or a cannabinoid include downregulation, disruption, or deletion of genes encoding alcohol dehydrogenase, lactate dehydrogenase, acetyl phosphate transferase and acetate kinase enzymes that divert acetate and acetyl-CoA to ethanol, lactate, and acetyl phosphate. In an E. coli host cell, genes that are downregulated, disrupted, or deleted can include adhE, ldhA, and ack-pta. Further, a cell engineered for the production of olivetolic acid or a cannabinoid can have one or more genes encoding thioesterases downregulated, disrupted, or deleted to prevent hydrolysis of olivetolic acid precursors hexanoyl-CoA and malonyl-CoA (as well as acetyl-CoA). For example, in an E. coli host one or more of the thioesterases genes FadM (P_414977.1), TesA (P_415027.1), TesB (P_414986.1), YciA (P_415769.1), Ydil (P_416201.1) and YbgC (P_415264.1) can be downregulated, disrupted, or deleted. In various embodiments a culture of a host cell engineered to have reduced or eliminated activity of one or more endogenous thioesterases can include hexanol or another fatty alcohol (including a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids) or hexanoate or another fatty acids (including propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids) or alkanoate provided in the culture medium during at least a portion of the time the cells are producing olivetolic acid, a derivative thereof, or a cannabinoid.

Reducing fatty acid degradation may also be desirable for reducing degradation of the hexanoyl-CoA (or other acyl-CoA) precursor. In E. coli for example, the fadE gene (encoding acyl-CoA dehydrogenase) can be downregulated, disrupted, or deleted. Other modifications to endogenous genes that may improve olivetolic acid or cannabinoid synthesis include mutation or downregulation of one or more genes that function in fatty acid biosynthesis. Without limiting the embodiments to any particular mechanism, limiting fatty acid biosynthesis can increase the malonyl-CoA supply available for cannabinoid biosynthesis. In some embodiments, a temperature-sensitive variant of the enoyl-CoA reductase (E. coli FabI) can be engineered into a host cell to result in diminished fatty acid biosynthesis at the non-permissive temperature. Another example of a fatty acid biosynthesis gene of a host cell that may be mutated or downregulated is a gene encoding a beta-ketoacyl-ACP synthase I enzyme (E. coli FabB). Other fatty acid biosynthesis endogenous genes of the engineered host cell that can be mutated to result in an enzyme of diminished function and thereby increase the malonyl-CoA supply include the gene beta-ketoacyl-ACP synthase III (E. coli FabH) and the gene encoding malonyl-CoA-ACP transacylase (E. coli FabD). In some examples, a gene encoding a beta-ketoacyl-ACP synthase II enzyme (E. coli FabF) can be overexpressed to reduce fatty acid biosynthesis.

Alternatively or in addition, genes encoding enzymes of the tricarboxylic acid cycle, such as succinate dehydrogenase, or the gene encoding citrate synthase that converts acetyl-CoA to citrate, which feeds into the TCA, can be disrupted or downregulated.

Engineered cells that produce a cannabinoid or olivetolic acid or a derivative thereof, can be engineered to include multiple pathways to enhance olivetolic acid or cannabinoid production. Those skilled in the art will recognize that the embodiments described herein can be combined in multiple ways. Examples of engineered cells having multiple genetic modifications are exemplary only and do not limit the scope of the invention.

### Host Cells

A host cell as provided herein can be a prokaryotic cell or a eukaryotic cell. Eukaryotic cells may be microbial eukaryotic cells, such as, for example, fungal cells or microalgal cells. Further, a eukaryotic cell engineered to produce at least one cannabinoid can be a cell or cell line derived from a multicellular eukaryote, such as but not limited to an alga, moss, or higher plant. Prokaryotic cells that can be engineered as provided herein include bacterial cells, archaebacterial cells, and cyanobacterial cells.

In some embodiments, a host cell is a microorganism such as a bacteria, filamentous fungus, or yeast. Host can be selected based on their ability to take up and utilize particular carbon sources, nitrogen sources, or precursor molecules or may be engineered to take up and utilize molecules that may be added to the culture medium.

Nonlimiting examples of suitable microbial hosts for the bio-production of a cannabinoid include, but are not limited to, any gram negative organisms, more particularly a member of the family Enterobacteriaceae, such as E. coli, or Oligotropha carboxidovorans, or a Pseudomononas sp.; any gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. More particularly, suitable microbial hosts for the bio-production of olivetolic acid or at least one cannabinoid generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, and Saccharomyces. Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

A variety of microorganism may be suitable for the production of cannibinoids in cell culture. Such organisms include both prokaryotic and eukaryotic organisms including, but not limited to, bacteria, including archaea and eubacteria, and eukaryotes, including yeast, plant, insect, animal, and mammal, including human. Exemplary species are reported in U.S. application Ser. No. 13/975,678 (filed Aug. 26, 2013), which is incorporated herein by reference, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain embodiments, suitable organisms include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobiwn denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobiurn loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella therrnoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus solaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana, Dunaliella salina, Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scvtonema, Spirulina, Stanieria, Starria, Stigonema, Symploca, Synechococcus, Svnechocystis, Tolipothrix, Trichodesmium, Tychonema, and Xenococcus species.

The microalgae host cells can produce a storage oil, which in some embodiments can include hydrocarbons such as triacylglyceride that may be stored in storage bodies of the host cell as well as related products that can include, without limitation, phospholipids, tocopherols, tocotrienols, carotenoids (e.g., alpha-carotene, beta-carotene, lycopene, etc.), xanthophylls (e.g., lutein, zeaxanthin, alpha-cryptoxanthin and beta-crytoxanthin), cannabinoids, isoprenoids and various organic or inorganic compounds. A raw oil may be obtained from the cells by disrupting the cells and isolating the oil. See WO2008/151149, WO2010/06032, WO2011/150410, and WO2011/1504 which disclose heterotrophic cultivation and oil isolation techniques, and all of which are incorporated by reference in their entirety for all purposes. For example, oil may be obtained by cultivating, drying and pressing the cells. The oils produced may also be refined, bleached and deodorized (RBD) to remove phospholipids, free fatty acids and odors as known in the art or as described in WO2010/120939, which is incorporated by reference in its entirety for all purposes. The raw or RBD oils may be used in a variety of food, chemical, pharmaceutical, nutraceutical and industrial products or processes. After recovery of the oil, a valuable residual biomass remains. Uses for the residual biomass can include the production of paper, plastics, absorbents, adsorbents, as animal feed, for human nutrition, or for fertilizer.

The stable carbon isotope value 513C is an expression of the ratio of 13C/12C relative to a standard (e.g. PDB, carbonite of fossil skeleton of Belemnite americana from Peedee formation of South Carolina). The stable carbon isotope value 513C (0/00) of the oils can be related to the 513C value of the feedstock used. The oils can be derived from oleaginous organisms heterotrophically grown, for example, on sugar derived from a C4 plant such as corn or sugarcane. The 513C (0/00) of the oil can be from −10 to −17 0/00 or from −13 to −16 0/00.

The oils disclosed herein can be made by methods using a microalgal host cell. As described above, the microalga can be, without limitation, Chlorophyta, Trebouxiophyceae, Chlorellales, Chlorellaceae, or Chlorophyceae. It has been found that oils from microalgae of Trebouxiophyceae can be distinguished from vegetable oils based on their sterol profiles. Oil produced by Chlorella protothecoides can include sterols such as brassicasterol, ergosterol, campesterol, stigmasterol, and β-sitosterol. Sterols produced by Chlorella can have C24 stereochemistry. Microalgae oils can also include, for example, campesterol, stigmasterol, β-sitosterol, 22,23-dihydrobrassicasterol, proferasterol and clionasterol. Oils produced by the microalgae may be distinguished from plant oils by the presence of sterols with C24 stereochemistry and the absence of C24a stereochemistry in the sterols present. For example, the oils produced may contain 22,23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ7-poriferasterol.

Oleaginous host cells engineered for production of cannabinoids as provided herein can produce an oil with at least 1% of cannabinoid. The oleaginous host cell (e.g., microalgae) can produce an oil, cannabinoid, triglyceride, isoprenoid or derivative of any of these. These host cells can be made by transforming a cell with any of the nucleic acids discussed herein. The transformed cell can be cultivated to produce an oil and, optionally, the oil can be extracted. Oil extracted can be used to produce food, oleochemicals, nutraceuticals, pharmaceuticals or other products.

The oils discussed above alone or in combination can be useful in the production of foods, pharmaceuticals, nutraceuticals, and chemicals. The oils, cannabinoids, isoprenoids, triglycerides can be subjected to decarboxilation, oxidation, light exposure, hydroamino methylation, methoxy-carbonation, ozonolysis, enzymatic transformations, epoxidation, methylation, dimerization, thiolation, metathesis, hydro-alkylation, lactonization, or other chemical processes. After extracting the oil, a residual biomass may be left, which may have use as a fuel, as an animal feed, or as an ingredient in paper, plastic, or other product.

The ability to genetically modify the host is essential for any recombinant production system. The mode of gene transfer technology may be by electroporation, conjugation, transduction or natural transformation.

The host cells or microorganisms of the disclosure include host strains or host cells that are genetically engineered to include genetic alterations designed to improve the rate, yield, or titer of cannabinoid production by cell cultures. Various optional genetic manipulations and alterations can be used interchangeably from one host cell to another, depending on the native enzymatic pathways present in the selected host cell.

To genetically modify a parent host cell to produce a genetically modified host cell of the present disclosure, one or more heterologous nucleic acids disclosed herein is introduced stably or transiently into a host cell, using established techniques. Such techniques may include, but are not limited to, electroporation, calcium phosphate precipitation, DEAE-dextran mediated transfection, liposome-mediated transfection, particle bombardment, and the like. For stable transformation, a heterologous nucleic acid will generally further include a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available. The cloning vectors are tailored to the host organisms based on the nature of antibiotic resistance markers that can function in that host.

One or more nucleic acid sequences disclosed herein can be present in an expression vector or construct. Suitable expression vectors may include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). Thus, for example, one or more nucleic acids encoding a cannabinoid pathway gene product is included in any one of a variety of expression vectors for expressing the cannabinoid pathway gene product(s). Such vectors may include chromosomal, non-chromosomal, and synthetic DNA sequences. Numerous additional suitable expression vectors are known to those of skill in the art, and many are commercially available. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

In some embodiments, a parent host cell is genetically modified to produce a genetically modified host cell of the present disclosure using a CRISPR/Cas9 or other CRISPR system to genetically modify a parent host cell, for example, with one or more heterologous nucleic acids disclosed herein.

In some instances, a chemically synthesized or PCR-amplified nucleic acid fragment, or a nucleic acid fragment excised from a larger nucleic acid molecule or construct, can be introduced into a host cell and optionally integrated into a nucleic acid molecule of the host cell, for example, using CRISPR technology. A nucleic acid fragment introduced into a host cell may or may not include a selectable marker, and may or may not include an expression cassette. For example, a nucleic acid fragment introduced into a host cell for cas9 engineering (or engineering via another RNA-guided endonuclease, e.g., Cpf1) can optionally include the coding sequence of a gene or a portion thereof in the absence of a promoter sequence, or alternatively, may include a promoter sequence or portion thereof in the absence of a complete coding sequence linked to the promoter sequence. Vectors, constructs, and nucleic acid fragments designed for introduction into a host cell can in some embodiments optionally include sequences for mediating homologous recombination into a host chromosome or episome.

Heterologous natural or chemically synthesized genes for enzymes may be introduced on high-level expression plasmid vectors or through genomic integration using methods well known to those skilled in the art. Such methods may involve CRISPR technology. Alternatively, genes that are endogenous to the host organism may be up-regulated by genetic element integration methods known to those skilled in the art.

In some embodiments, one, two, three, four, or more of the nucleic acid sequences disclosed herein that encode an enzyme or other polypeptide that functions in a pathway for producing a cannabinoid or OA or a derivative thereof, or a pathway that reduces byproduct formation, are present in a single expression vector or construct. In some embodiments, two, three, four or more nucleic acid sequences disclosed herein that encode an enzyme or other polypeptide that functions in a pathway for producing a cannabinoid or OA or a derivative thereof, or a pathway that reduces byproduct formation, are present in are in separate expression vectors or constructs. In some embodiments, one, two, three, four, or more nucleic acid sequences that encode an enzyme or other polypeptide that functions in a pathway for producing a cannabinoid or OA or a derivative thereof, or a pathway that reduces byproduct formation, are integrated into a chromosome or episome using an RNA-guided nuclease such as a CRISPR RNA-guided nuclease. Multiple genes encoding enzymes can be inserted into the host chromosome or episome individually, for example, sequentially, or multiple genes may be in inserted into the host chromosome or episome together.

Promoters used for driving transcription of genes in S. cerevisiae and other yeasts are well known in the art and include DNA elements that are regulated by glucose concentration in the growth media, such as the alcohol dehydrogenase-2 (ADH2) promoter. Other regulated promoters or inducible promoters, such as those that drive expression of the GAL1, MET25 and CUP1 genes, are used when conditional expression is required. GAL1 and CUP1 are induced by galactose and copper, respectively, whereas MET25 is induced by the absence of methionine. In some embodiments, one or more of the exogenous polynucleotides is operably linked to a glucose regulated promoter. In some embodiments, expression of one or more of the exogenous polynucleotides is driven by an alcohol dehydrogenase-2 promoter. Other promoters drive strongly transcription in a constitutive manner. Such promoters include, without limitation, the control elements for highly expressed yeast glycolytic enzymes, such as glyceraldehyde-3-phosphate dehydrogenase (GPD), phosphoglycerate kinase (PGK), pyruvate kinase (PYK), triose phosphate isomerase (TPI) and alcohol dehydrogenase-1 (ADH1). Another strong constitutive promoter that may be used is that from the S. cerevisiae transcription elongation factor EF-1 alpha gene (TEF1) (Partow et al., Yeast. 2010, (11):955-64). Promoters for engineering of bacterial hosts are well-known in the art and include inducible promoters such as example, the ara, lac, trc, tet, and cumate-regulated promoters.

In the above embodiments, the nucleic acid sequences may optionally be chemically synthesized genes, with codon optimization for the host being genetically engineered, that encode a wild type or mutant enzyme from another species or the host species.

Engineering of a host cells as provided herein can include expressing a variant of a naturally-occurring enzyme in the host cell. For making variants, mutagenesis methods are well known in the art and include, for example, error-prone PCR (Leung et al. (1989) Technique 1:11-15; and Caldwell et al. (1992) PCR Methods Applic. 2:28-33), oligonucleotide directed mutagenesis (Reidhaar-Olson et al. (1988) Science 241:53-57), assembly PCR (U.S. Pat. No. 5,965,408), and sexual PCR mutagenesis (Stemmer (1994) PNAS, USA 91:10747-10751. Cassette mutagenesis can be used to generate mutant proteins (Richards, J. H. (1986) Nature 323:187; Ecker et al. (1987) J. Biol. Chem. 262:3524-3527); to insert or replace individual codons (Kegler-Ebo et al. (1994) Nucleic Acids Res. 22(9):1593-1599), or to make variants of sequences comprising regulatory sequences (e.g., ribosome binding sites, see, e.g., Barrick et al. (1994) Nucleic Acids Res. 22(7):1287-1295); Wilson et al. (1994) Biotechniques 17:944-953). Recursive ensemble mutagenesis (Arkin et al. (1992) PNAS, USA 89:7811-7815) or exponential ensemble mutagenesis (Delegrave et al. (1993) Biotech. Res. 11:1548-1552) can also be used to generate nucleotide sequence variants. Random and site-directed mutagenesis can also be used (Arnold (1993) Curr. Opin. Biotech. 4:450-455).

Variants of enzymes of interest can also be created by in vivo mutagenesis. In some embodiments, random mutations in a nucleic acid sequence are generated by propagating the polynucleotide sequence in a bacterial strain, such as an E. coli strain, which carries mutations in one or more of the DNA repair pathways. Such “mutator” strains have a higher random mutation rate than that of a wild-type strain. Propagating a DNA sequence in one of these strains will eventually generate random mutations within the DNA. Mutator strains suitable for use for in vivo mutagenesis are described in, for example, PCT International Publication No. WO 91/16427. Standard methods of in vivo mutagenesis can be used. For example, host cells, comprising one or more polynucleotide sequences that include an open reading frame for an ACC polypeptide, as well as operably-linked regulatory sequences, can be subject to mutagenesis via exposure to radiation (e.g., UV light or X-rays) or exposure to chemicals (e.g., ethylating agents, alkylating agents, or nucleic acid analogs). In some host cell types, for example, bacteria, yeast, and plants, transposable elements can also be used for in vivo mutagenesis.

The cannabinoid-producing engineered cells of the invention may be made by transforming a host cell, either through genomic integration or using episomal plasmids (also referred to as expression vectors, or simply vectors) with at least one nucleotide sequence encoding enzymes involved in the engineered metabolic pathways. As used herein the term “nucleotide sequence” and “nucleic acid sequence” are used interchangeably and mean a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA. In some embodiments, the nucleotide sequence is codon-optimized to reflect the typical codon usage of the host cell without altering the polypeptide encoded by the nucleotide sequence. In certain embodiments, the term “codon optimization” or “codon-optimized” refers to modifying the codon content of a nucleic acid sequence without modifying the sequence of the polypeptide encoded by the nucleic acid to optimize expression in a particular host cell.

Methods of introducing exogenous nucleic acids into plant cells are also well known in the art. Such plant cells are considered “transformed.” Suitable methods may include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, CRISPR/Cas9-mediated genome editing, and the like. The choice of method is generally dependent on the type of cell being transformed and the circumstances under which the transformation is taking place (e.g., in vitro, ex vivo, or in vivo).

In other aspects, engineering may be employed to reduce the production of byproducts, e.g., ethanol that utilize carbon sources that lead to reduced utilization of that carbon source for cannabinoid production. Such genes may be completely “knocked out” of the genome by deletion, or may be reduced in activity through reduction of promoter strength or the like. Such genes include those for the enzymes alcohol dehydrogenase and lactate dehydrogenase, for example.

Given the teachings and guidance provided herein, those skilled in the art will understand that enzymatic activity or expression can be attenuated using well known methods. Reduction of the activity or amount of an enzyme can mimic complete disruption of a gene if the reduction causes activity of the enzyme to fall below a critical level that is normally required for a pathway to function. Reduction of enzymatic activity by various techniques rather than use of a gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme activity or kinetics (see Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999). Natural or imposed regulatory controls can also accomplish enzyme attenuation including: promoter replacement (see Wang et al., Mol. Biotechnol. 52(2):300-308 (2012)); loss or alteration of transcription factors (Dietrick et al., Annu. Rev. Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as an enzyme inhibitor, an antibiotic or a target-specific drug.

One skilled in the art will also understand and recognize that attenuation of an enzyme can be done at various levels. For example, at the gene level, a mutation causing a partial or complete null phenotype, such as a gene disruption or a mutation causing epistatic genetic effects that mask the activity of a gene product (Miko, Nature Education 1(1) (2008)), can be used to attenuate an enzyme. At the gene expression level, methods for attenuation include: coupling transcription to an endogenous or exogenous inducer such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in region in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell, 136(4):763-776 (2009); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112 (2013); and Kawasaki et al., Curr. Opin. Mol. Ther. 7(2):125-131 (2005); modifying regions outside the coding sequence, such as introducing secondary structure into an untranslated region (UTR) to block translation or reduce efficiency of translation (Ringnér et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932 (2010); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012). At the level of enzyme localization and/or longevity, enzyme attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme being secreted or localized to a subcellular compartment in a eukaryotic cell, where the enzyme would not be able to react with its normal substrate Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261 (2003)). At the level of enzyme activity, enzyme attenuation can include: adding an endogenous or an exogenous inhibitor, such as an enzyme inhibitor, an antibiotic, or a target-specific drug, to reduce enzyme activity; limiting availability of essential cofactors, such as vitamin B12, for an enzyme that requires the cofactor; chelating a metal ion that is required for enzyme activity; or introducing a dominant negative mutation. The applicability of a technique for attenuation described above can depend upon whether a given host microbial organism is prokaryotic or eukaryotic, and it is understood that a determination of what is the appropriate technique for a given host can be readily made by one skilled in the art.

A CRISPR/Cas9 (or other RNA-guided endonuclease) system can be used to generate a transgenic (genetically modified) microorganism or plant cell of the present disclosure, including generating regulatory mutants (e.g., “knockdown” or decreased expression of endogenous genes) and knockout mutations. CRISPR/Cas9 and other CRISPR systems and methods for mutating promoters, causing insertions in the upstream regions of genes that negatively affect gene expression, and disrupting genes are also known in the art. See, e.g., Bortesi and Fischer (2015) Biotechnol. Advances 33:41; Fan et al. (2015) Sci. Reports 5:12217; Ajjawi et al. (2017) Nature Biotech 35:647-652.

In yet another aspect, methods for producing olivetolic acid, a derivative of olivetolic acid, or a cannabinoid that include incubating a culture of an engineered host cell as provided herein to produce olivetolic acid, a derivative of olivetolic acid, or a cannabinoid. The methods can further include recovering the olivetolic acid, or cannabinoid from the cells, the culture medium, or whole culture.

The cultures comprise cells engineered for the production of cannabinoids in a culture medium. In various embodiments the engineered host cells can be bacterial, fungal, or algal cells, including cyanobacterial and eukaryotic microalgal cells. In embodiments where the cells are heterotrophic cells, the culture medium includes at least one carbon source that is also an energy source. The culture medium can include one, two, three, or more carbon sources that are not primary energy sources. Nonlimiting examples of feed molecules that can be included in the culture medium include acetate, malonate, oxaloacetate, aspartate, glutamate, beta-alanine, alpha-alanine, hexanoate, hexanol, prenol, isoprenol, and geraniol. Further examples of compounds that can be provided in the culture medium include, without limitation, biotin, thiamine, pantotheine, and 4-phosphopantetheine.

In some embodiments, acetate is provided in the culture medium. In some embodiment, acetate and hexanoate are provided in the culture medium. In some embodiments, malonate and hexanoate are provided in the culture medium. In either of these embodiments, the culture medium can further include prenol, isoprenol, or geraniol. In some embodiments, aspartate, hexanoate, and prenol, isoprenol, or geraniol are present in the culture medium.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used here, culture medium, or simply “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements. “Whole culture” as used herein refers to cultured cells plus the culture medium they are cultured in.

Exemplary carbon sources include sugar carbons such as sucrose, glucose, galactose, fructose, mannose, isomaltose, xylose, pannose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, culture conditions include aerobic, microaerobic, anaerobic or substantially anaerobic growth or maintenance conditions. Exemplary aerobic, microaerobic, and anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large scale culture procedures. Useful yields of the products can be obtained under aerobic, microaerobic, anaerobic or substantially anaerobic culture conditions.

Algae can be cultured photoautotrophically, in the light, without a reduced carbon source that can be used for energy, mixotrophically, where the algae are exposed to light that allows photosynthesis and also use a reduced carbon source provided in the culture medium, or heterotrophically, in the dark, where the cells rely entirely on a reduced carbon source provided in the culture medium for growth and energy.

An exemplary growth condition for achieving, one or more cannabinoid product(s) includes aerobic, microaerobic, anaerobic culture or fermentation conditions. In certain embodiments, the microbial organism can be sustained, cultured or fermented under aerobic, microaerobic, anaerobic or substantially anaerobic conditions. Briefly, anaerobic conditions refer to an environment devoid of oxygen. Conditions include, for example, a culture, batch fermentation or continuous fermentation such that the dissolved oxygen concentration in the medium remains between 0 and 10% of saturation, or higher. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art. Typically cells are grown at a temperature in the range of about 25° C. to about 40° C. in an appropriate medium, as well as up to 70° C. for thermophilic microorganisms.

The culture medium may include a feed molecule that is converted into a cannabinoid precursor, such as, but not limited to, CO₂, acetate, malonate, beta-alanine, aspartate, glutamate, oxaloacetate, hexanoate, hexanol, prenol, isoprenol, or geraniol. The feed molecule can also serve as the main or a supplemental carbon source for cell growth and energy, or can be provided in addition to a sugar, sugar alcohol, polyol, or organic acid that is provided for growth and energy. Additional supplements can optionally include biotin, thiamine, pantothenate, and/or 4′-phosphopantotheine.

The culture medium at the start of fermentation may have a pH of about 5 to about 7. The pH may be less than 11, less than 10, less than 9, or less than 8. In other embodiments the pH may be at least 2, at least 3, at least 4, at least 5, at least 6, or at least 7. In other embodiments, the pH of the medium may be about 6 to about 9.5; 6 to about 9, about 6 to 8 or about 8 to 9.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

Suitable purification and/or assays to test, e.g., for the production of olivetolic acid or a cannabinoid can be performed using well known methods. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

Olivetolic acid or a cannabinoid may separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other product(s), including a polyketide, produced in a bio-production media generally can be determined using any of methods such as, for example, high performance liquid chromatography (HPLC), gas chromatography (GC), GC/Mass Spectroscopy (MS), or spectrometry. All of the above methods are well known in the art.

The disclosure also provides compositions that are enriched for desired cannabinoids and derivatives thereof, for example, CBGA or any of the cannabinoids disclosed herein. Such enriched compositions include those that are pharmaceutical compositions as well as those that are used for non-pharmaceutical purposes, including medicinal purposes. Accordingly, in some embodiments, provided are compositions, such as pharmaceutical compositions or medicinal compositions, with CBGA and/or CBG that are 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater or even 100% CBGA or its decarboxylated derivative CBG, of all olivetolic acid, olivetolic acid derivative, and cannabinoid compounds.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art.

The combinations set forth herein are not intended to be limiting. Any of the embodiments of modifications of engineered cells, culture compositions, and methods of producing olivetolic acid, a derivative of olivetolic acid, or a cannabinoid may be combined within the scope of the invention.

### EXAMPLE 1

### Production of OLA in E Coli Engineered with OLA Pathway

An E coli strain derived from MG1655 was transformed with three plasmids to produce OLA. Plasmid A is derived froin pET-28 (Addgene) and expresses OLS and OAC under a cumate-inducible promoter; plasmid B is derived from pCDF (Addgene) and expresses accABCD and fadD from E coli under a T7 promoter; plasmid C is derived from pZS23S (Expressys) and expresses a T7 RNA polymerase under an IPTG-inducible promoter. Cells of an OD 3 were cultured in a 48-well plate at 30 degree for 25 hours with a shaking speed of 400 RPM in minimal medium supplied with trace element, manganese, thymine, biotin, cumate, IPTG, and hexanoate. Cell cultures were spun down for 10 min and medium broth was analyzed for OLA. The strain was able to produce 687 uM OLA.

### EXAMPLE 2

### Production of CBGA in E Coli with OLA and Prenol/Isoprenol Mix

An E coli strain derived from MG1655 was engineered to overexpress a GPP synthase (idsA), prenol kinase thiM, IP/DMAP kinase IPK, DMAPP/IPP isomerase idi, and prenyltransferase NphB to produce CBGA when substrates prenol and OLA were supplied externally. idsA, thiM, IPK, and idi were overexpressed on the chromosome while nphB was overexpressed on a plasmid derived from pZS13S (Expressys). Cells were cultured in a 48-well plate at 30 degree for 47 hours with a shaking speed of 400 RPM in minimal medium supplied with trace element, manganese, thymine, 400 uM OLA, and 20 mM prenol/isoprenol mix. Cell cultures were extracted with acetonitrile and then spun down for 10 min. The supernant was analyzed for CBGA with LCMS. The strain produced CBGA ranging from 0.5 to 20.6 uM CBGA.

### EXAMPLE 3

### Production of CBGA in E. Coli with Hexanoate and Prenol Feed

An E coli strain derived from MG1655 was engineered to overexpress accABCD, fadD, OLS, OAC, GPP synthase, prenol kinase thiM, IP/DMAP kinase IPK, DMAPP/IPP isomerase idi, and prenyltransferase NphB to produce CBGA when substrates prenol and hexanoate were supplied externally. OLS, OAC, gpps, thiM, IPK, and idi were overexpressed on the chromosome while accABCD, fadD, and nphB were overexpressed on a plasmid. Cells were cultured in a 48-well plate at 30 degree for 25 hours with a shaking speed of 400 RPM in minimal medium supplied with trace element, manganese, thymine, biotin, IPTG, 4mM hexanoate, and 20 mM prenol. Cell cultures were extracted with acetonitrile and then spun down for 10 mM. The supernant was analyzed for CBGA with LCMS. The strain produced 1.4 uM CBGA.

1. An engineered cell engineered for the production of olivetolic acid, a derivative of olivetolic acid, or a cannabinoid, wherein the engineered cell is engineered to express or overexpress:
a. at least one nucleic acid sequence encoding a polypeptide having Type III polyketide synthase activity;
b. at least one nucleic acid sequence encoding a polypeptide sequence having olivetolic acid cyclase activity that forms olivetolic acid or an olivetolic acid derivative; and
at least one nucleic acid sequence encoding an acetyl CoA carboxylase (ACC) or subunit thereof.

2. The engineered cell according to claim 1, wherein the engineered cell is engineered to express or overexpress an ACC or subunit thereof, further wherein: the nucleotide sequence of the ACC or ACC subunit gene is refactored, the ACC or subunit thereof is a variant, and/or the ACC or subunit thereof is operably linked to a heterologous promoter.

3. The engineered cell according to claim 1, wherein the engineered cell produces more of olivetolic acid, an olivetolic acid derivative, or a cannabinoid than is produced by a control cell substantially identical to the engineered cell with the exception that the control cell is not engineered to express or overexpress a nucleic acid sequence as set forth in (c).

4. The engineered cell of claim 1, wherein the engineered cell is
(a) engineered to express or overexpress a nucleic acid sequence encoding a malonyl-CoA synthetase, or optionally further engineered to express or overexpress a nucleic acid sequence encoding a malonate transporter;
(b) engineered to express or overexpress a nucleic acid sequence encoding a methylmalonyl-CoA carboxytransferase, or optionally further engineered to express or overexpress a phosphoenolpyruvate carboxykinase (PEPCK) or further engineered to express or overexpress an aspartate aminotransferase; or
(c) engineered to express or overexpress a beta alanine pyruvate aminotransferase (BAPAT), or optionally further engineered to express or overexpress an aspartate decarboxylase or further engineered to express or overexpress a nucleic acid sequence encoding a malonyl-CoA reductase.

5-11. (canceled)

12. An engineered cell engineered for the production of olivetolic acid, a derivative of olivetolic acid, or a cannabinoid, wherein the engineered cell is engineered to express an exogenous gene encoding a CoA ligase having activity on acetate, hexanoate or other fatty acid.

13. The engineered cell according to claim 12, wherein
(a) the CoA ligase comprises SEQ ID NO: 89-95, or a homolog or variant thereof having at least 50% identity thereto;
(b) the cell is engineered to express or overexpress at least one gene encoding an acetyl-CoA carboxylase or a subunit thereof; or
(c) the engineered cell produces more of olivetolic acid, an olivetolic acid derivative, or a cannabinoid than is produced by a control cell substantially identical to the engineered cell with the exception that the control cell is not engineered to express an exogenous gene encoding an acyl-CoA ligase having activity on acetate and hexanoate.

14. (canceled)

15. (canceled)

16. The engineered cell according to claim 1, wherein the engineered cell further comprises one or more modifications selected from the group consisting of:
d. expression of an exogenous gene or over-expression of an endogenous gene encoding a lactonase;
e. disruption or downregulation of an endogenous gene encoding a lactone transporter;
f. expression of an exogenous gene encoding a farnesol kinase;
g. expression of an exogenous gene encoding an isopentenyl phosphate kinase;
h. disruption or downregulation of an endogenous gene encoding encoding a nudix hydrolase, an alkaline phosphatase, a diacylglycerol diphosphate phosphatase, and/or a lipid phosphate phosphatase; and
i. expression of an exogenous gene or over-expression of an endogenous gene encoding an alcohol dehydrogenase that converts hexanol to hexanal or to hexanoyl-CoA or converts a fatty alcohol to its fatty aldehyde or to its fatty acyl-CoA, or expression of an exogenous gene or over-expression of an endogenous gene encoding an aldehyde dehydrogenase that converts hexanal to hexanoyl-CoA or converts a fatty aldehyde to its fatty acyl-CoA.

17. (canceled)

18. The engineered cell according to claim 1, wherein the engineered cell is further modified to express or overexpress a nucleic acid sequence encoding a prenyltransferase.

19. The engineered cell according to claim 1, wherein the engineered cell is further modified to express or overexpress a nucleic acid sequence encoding an alcohol kinase, an alcohol diphosphokinase, and/or a prenyl phosphate kinase.

20. The engineered cell according to claim 1, wherein the engineered cell is further modified to express or overexpress a nucleic acid sequence encoding a geranyl phosphate kinase, a farnesol kinase, or a geraniol kinase.

21. An engineered cell engineered for the production of a cannabinoid, wherein the engineered cell is engineered to express or overexpress:
a. at least one nucleic acid sequence encoding a polypeptide having Type III polyketide synthase activity;
b. at least one nucleic acid sequence encoding a polypeptide sequence having olivetolic acid cyclase activity; and
c. at least one nucleic acid sequence encoding a nucleic acid sequence encoding an alcohol kinase, an alcohol diphosphokinase, and/or a prenyl phosphate kinase.

22. The engineered cell according to claim 1, wherein the engineered cell is further modified to express or overexpress a nucleic acid sequence encoding a GPP Synthase.

23. The engineered cell of claim 1, wherein the engineered cell is modified to have reduced expression of one or more of: a gene encoding an alcohol dehydrogenase, a gene encoding a lactate dehydrogenase, an acetyl phosphate transferase, an acetate kinase, a gene encoding a thioesterase, a gene encoding a fatty acid biosynthesis gene, or a fatty acid degradation gene.

24. The engineered cell of claim 1, wherein the cell is further engineered to include a reverse beta oxidation pathway.

25. A cell culture comprising an engineered cell of claim 1.

26. The cell culture of claim 25 comprising acetate, malonate, or both acetate and malonate.

27. (canceled)

28. A cell culture comprising the cell of claim 4 and (a) oxaloacetate, aspartate, or glutamate, or (b) oxaloacetate, aspartate, or beta-alanine.

29. (canceled)

30. The cell culture according to claim 25 comprising (a) prenol or isoprenol or a mixture of prenol and isoprenol, or (b) geraniol.

31. (canceled)

32. The cell culture according to claim 25 comprising a fatty acid.

33-37. (canceled)

38. A method of producing olivetolic acid, an olivetolic acid derivative, or a cannabinoid comprising culturing a engineered cell according to claim 1 in a culture medium to produce olivetolic acid, an olivetolic acid derivative, or a cannabinoid.

39. (canceled)
